(19) United States
(12) Patent Application Publication
(10) Pub. No.: US 2023/0002788 A1
(43) Pub. Date: Jan. 5, 2023

(54) AAV3B VARIANTS WITH IMPROVED PRODUCTION YIELD AND LIVER TROPISM

(71) Applicant: The Trustees of the University of Pennsylvania, Philadelphia, PA (US)

(72) Inventors: James M. Wilson, Philadelphia, PA (US); Qiang Wang, Philadelphia, PA (US)

(21) Appl. No.: 17/770,138

(22) PCT Filed: Oct. 20, 2020
(86) PCT No.: PCT/US2020/056511

§ 371 Date: Apr. 19, 2022

Related U.S. Application Data
(60) Provisional application No. 63/025,753, filed on May 15, 2020.
(60) Provisional application No. 62/924,112, filed on Oct. 21, 2019.

(51) Int. Cl. C12N 15/861

(52) U.S. Cl. CPC C12N 15/861; C12N 2750/14143; C12N 2750/14122

(57) ABSTRACT

Provided herein are novel AAV capsids and rAAV comprising the same. In one embodiment, vectors employing the AAV capsid show increased transduction in a selected tissue as compared to a prior art AAV.

### STATEMENT OF GOVERNMENT SUPPORT

This invention was made with government support under grant number P01HL059407 awarded by the National Institutes of Health. The government has certain rights in the invention.

### BACKGROUND OF THE INVENTION

Adeno-associated virus (AAV) vectors hold great promise in human gene therapy and have been widely used to target liver, muscle, heart, brain, eye, kidney, and other tissues in various studies due to their ability to provide long-term gene expression and lack of pathogenicity. AAV belongs to the parvovirus family and contains a single-stranded DNA genome flanked by two inverted terminal repeats. Dozens of naturally occurring AAV capsids have been reported; their unique capsid structures enable them to recognize and transduce different cell types and organs.

Since the first trial which started in 1981, there has not been any vector-related toxicity reported in clinical trials of AAV vector-based gene therapy. The ever-accumulating safety records of AAV vector in clinical trials, combined with demonstrated efficacy, show that AAV is an attractive platform. In particular, AAV is easily manipulated as the virus has a single-stranded DNA virus with a relatively small genome (˜4.7 kb) and simple genetic components—inverted terminal repeats (ITR), the Rep and Cap genes. Only the ITRs and AAV capsid protein are required in AAV vectors, with the ITRs serving as replication and packaging signals for vector production and the capsid proteins playing a central role by forming capsids to accommodate vector genome DNA and determining tissue tropism.

AAV are among the most effective vector candidates for gene therapy due to their low immunogenicity and non-pathogenic nature. However, despite allowing for efficient gene transfer, the AAV vectors currently used in the clinic can be hindered by preexisting immunity to the virus and restricted tissue tropism. Thus, additional AAV vectors are needed.

### SUMMARY OF THE INVENTION

Engineered AAV3B capsids are provided which are useful for generating rAAV vectors for delivery of a gene product. The rAAV are particularly well-suited for human delivery, but may also be utilized in non-human animals, including, e.g., dogs and cats. The rAAV may be in a composition used as a gene therapy product, for gene editing, as a vaccine, amongst other suitable uses.

In one embodiment, the engineered capsid has the amino acid sequence of AAV3B.AR2.08 (SEQ ID NO: 15). In another embodiment, the engineered capsid has the amino acid sequence of AAV3B.AR2.16 (SEQ ID NO: 29). Also provided are an AAV3B.AR2.08 nucleic acid sequence encoding SEQ ID NO: 16 for use in producing an AAV capsid in combination with a vector genome to form a rAAV3B.AR2.08 rAAV particle. In certain embodiments, the AAV3B.AR2.08 nucleic acid sequence has the sequence of SEQ ID NO: 16 or a sequence at least 90%, at least 95%, at least 97%, at least 98% or at least 99% identical thereto. In certain embodiments, an AAV3B.AR2.16 nucleic acid sequence encoding SEQ ID NO: 30 is provided for use in producing an AAV capsid in combination with a vector genome to form a rAAV3B.AR2.16 rAAV particle. In certain embodiments, the AAV3B.AR2.16 nucleic acid sequence has the sequence of SEQ ID NO: 30 or a sequence at least 90%, at least 95%, at least 97%, at least 98% or at least 99% identical thereto.

Provided herein is a recombinant adeno-associated virus (rAAV) with a capsid having the sequence of AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), or AAV3B.AR2.17 (SEQ ID NO: 31), and having packaged in the capsid a vector genome comprising a non-AAV nucleic acid sequence.

Provided herein is a rAAV having a capsid encoded by the sequence of AAV3B.AR2.01 (SEQ ID NO: 2), AAV3B.AR2.02 (SEQ ID NO: 2), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), or AAV3B.AR2.17 (SEQ ID NO: 32), or a sequence sharing at least 90%, at least 95%, at least 97%, at least 98% or at least 99% identical to any of SEQ ID NO: 2, 4, 6, 8, 10, 12, 14, 18, 20, 22, 24, 26, 28, or 32, and having packaged in the capsid a vector genome comprising a non-AAV nucleic acid sequence.

Provided herein is a rAAV having a capsid encoded by SEQ ID NO: 2, 4, 6, 8, 10, 12, 14, 16, 18, 20, 22, 24, 26, 28, 30, or 32. In certain embodiments, the rAAV according has AAV inverted terminal repeats and a heterologous nucleic acid sequence operably linked to regulatory sequences which direct expression of a product encoded by the heterologous nucleic acid sequence in a target cell. In certain embodiments, the ITRs are from a different AAV than the AAV supplying the capsid. In certain embodiments, the ITRs are from AAV2.

Provided herein is a rAAV comprising (A) a capsid comprising one or more of: capsid proteins comprising: a heterogeneous population of vp1 proteins, a heterogeneous population of vp2 proteins, a heterogeneous population of vp3 proteins; wherein: the vp1, vp2 and vp3 proteins contain subpopulations with amino acid modifications comprising at least two highly deamidated asparagines (N) in asparagine-glycine pairs and, optionally, further comprising subpopulations comprising other deamidated amino acids, wherein the deamidation results in an amino acid change; and (B) a vector genome in the capsid, the vector genome comprising a nucleic acid molecule comprising AAV inverted terminal repeat sequences and a non-AAV nucleic acid sequence encoding a product operably linked to sequences which direct expression of the product in a host cell.

A method of transducing liver tissue by administering an rAAV having a capsid selected from AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.08 (SEQ ID NO: 15), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), AAV3B.AR2.16 (SEQ ID NO: 29), or AAV3B.AR2.17 (SEQ ID NO: 31) is provided herein.

Provided herein is a method of generating a rAAV with an AAV capsid including the steps of culturing a host cell containing: (a) a molecule encoding an AAV capsid protein of AAV3B.AR2.01 (SEQ ID NO: 2), AAV3B.AR2.02 (SEQ ID NO: 2), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.08 (SEQ ID NO: 16), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), AAV3B.AR2.16 (SEQ ID NO: 30), or AAV3B.AR2.17 (SEQ ID NO: 32), (b) a functional rep gene; (c) a minigene comprising AAV inverted terminal repeats (ITRs) and a transgene; and (d) sufficient helper functions to permit packaging of the minigene into the AAV capsid protein.

A composition comprising at least an AAV and a physiologically compatible carrier, buffer, adjuvant, and/or diluent is provided.

A method of delivering a transgene to a cell, the method comprising the step of contacting the cell with an rAAV is provided. In certain embodiments, the rAAV comprises a transgene.

Provided herein is a rAAV comprising an AAV capsid having an amino acid sequence selected from: AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.08 (SEQ ID NO: 15), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), AAV3B.AR2.16 (SEQ ID NO: 29), or AAV3B.AR2.17 (SEQ ID NO: 31), further comprising a non-AAV nucleic acid sequence.

Provided herein is a nucleic acid molecule comprising a nucleic acid sequence encoding an AAV capsid protein, wherein the nucleic acid sequence is selected from AAV3B.AR2.01 (SEQ ID NO: 2), AAV3B.AR2.02 (SEQ ID NO: 4), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.08 (SEQ ID NO: 16), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), AAV3B.AR2.16 (SEQ ID NO: 30), or AAV3B.AR2.17 (SEQ ID NO: 32). In certain embodiments, the nucleic acid molecule comprises an AAV sequence encoding an AAV capsid protein and a functional AAV rep protein. In certain embodiments, the nucleic acid molecule is a plasmid.

Provided herein is a host cell transfected with nucleic acid encoding an AAV capsid protein.

### BRIEF DESCRIPTION OF THE FIGURES

FIG. 1A-FIG. 1E show an alignment of the amino acid sequences of the AAV3B mutants described herein and the AAV3B native sequence.

FIG. 2A-FIG. 2S show an alignment of the nucleic acid sequences of the AAV3B mutants described herein and the AAV3B native sequence.

FIG. 3A-FIG. 3D show a workflow for the directed evolution platform. FIG. 3A and FIG. 3 describe the general process of AAV directed evolution. FIG. 3C is an illustration of the scorecard approach. The capsid sequences of around 180 natural AAVs were aligned and ten variable, surface-exposed sites within HVR. VIII were picked for mutagenesis. For each site, the amino acids with the highest frequencies according to the alignment were selected and incorporated into degenerate oligos for the mutagenesis. FIG. 3D describes the barcode evaluation system. A self-complementary AAV vector carrying a CB8.eGFP transgene cassette was used as the backbone for the barcode vectors. First, all the ATGs within the eGFP gene were eliminated. The 6-bp barcodes were then inserted after the eGFP gene. Each barcode vector was produced individually. The vectors were then pooled together before being injected into animals. Various tissues were collected and preserved at −80° C. in RNAlater solution. Next generation sequencing (NGS) was used to analyze the barcode frequencies in the PCR and RT-PCR products from those tissues.

FIG. 4A-FIG. 4M show the design and results of an evaluation of barcoded AAV3B variants injected into two NHP (B6134 and V208L) at a dosage of 2×10¹³ gc/kg IV. FIG. 4A and FIG. 4B show details relating to capsids evaluated, including AAV3B scorecard variants from two rounds of humanized FRG mouse selection. AAV3B and AAV8 were control capsids. Animal B6134 had an ALT elevation at the necropsy (day 7) (FIG. 4C). (FIG. 4D-FIG. 4M) Seven days post vector administration, tissues were harvested, and barcode fold changes were compared (shown relative to input, set as 1). For animal B6134, fold changes for each variant tested are shown in FIG. 4D and FIG. 4E (liver), FIG. 4F (heart and muscle), FIG. 4G (CNS), and FIG. 4H (other tissues). For animal V208L, fold changes for each variant tested are shown in FIG. 4I and FIG. 4J (liver), FIG. 4K (heart and muscle), FIG. 4L (CNS), FIG. 4M (other tissues). Columns are identified left to right. Lv: liver, LV C: caudate lobe, LV L: left lobe, Lu: lung, Lu: lung left upper, Mu: muscle, P: pancreas, K: kidney, xx.g: genomic DNA of xx, xx.R: xx right, xx.L: xx left, xx.RL: xx right lower, etc.

FIG. 5A-FIG. 5N show the design and results of an evaluation of barcoded AAV3B variants that were injected into two NHP (E499P and B4404) at a dosage of ˜1.8×10¹³ and ˜2.9×10¹³ gc/animal via intra-cisterna magna (ICM) injection. Details relating to injected vectors are shown in FIG. 5A and FIG. 5B. Fourteen days post vector administration, tissues were harvested. Barcode fold changes were compared. Fold changes in cortex and cerebellum are shown in FIG. 5C (normalized against variant input frequencies) and FIG. 5D (normalized against AAV3B) for animal E499P. Fold changes in hippocampus, striatum, and thalamus are shown in FIG. 5E (normalized against variant input frequencies) and FIG. 5F (normalized against AAV3B) for animal E499P. Fold changes in spinal cord are shown in FIG. 5G (normalized against variant input frequencies) and FIG. 5H (normalized against AAV3B) for animal E499P. Fold changes in cortex and cerebellum are shown in FIG. 5I (normalized against variant input frequencies) and FIG. 5J (normalized against AAV3B) for animal B4404. Fold changes in hippocampus, striatum, and thalamus are shown in FIG. 5K (normalized against variant input frequencies) and FIG. 5L (normalized against AAV3B) for animal B4404. Fold changes in spinal cord are shown in FIG. 5M (normalized against variant input frequencies) and FIG. 5N (normalized against AAV3B) for animal B4404. Columns are identified left to right.

FIG. 6A-FIG. 6D provide a comparison of AAV8 and AAV3B.AR2.16, with or without steroid treatment.

FIG. 7A provides genome copies of vector containing an hLDLR expression cassette in non-human primate liver at 18 days (d18) and 120 days (d120) following intravenous (iv) injection of 2.5×10¹³ GC/kg or 7.5×10¹² GC/kg vector.

FIG. 7B provides LDLR mRNA levels in liver.

FIG. 8A-FIG. 8F provide a comparison of AAV3B and engineered variants, with or without steroid co-therapy.

FIG. 9A provides genome copies of vector containing an hLDLR expression cassette in non-human primate liver at day 18, day 83/88 and day 120 following intravenous (iv) injection of 2.5×10¹³ GC/kg or 7.5×10¹² GC/kg vector.

FIG. 9B provides LDLR mRNA levels in liver.

FIG. 10A provides vector genome copies (GC) of the hLDLR expression cassette in livers in NHPs at day 18, day 83/88 and day 120.

FIG. 10B shows LDLR mRNA levels in the liver at the same time points for AAV3B and the two engineered variants. Liver biopsy was performed and the vector genome copies from the biopsy samples were measured by qPCR. The vector genome copies of the two variants are higher than AAV3B, indicating they are suitable candidates for liver gene therapy.

FIG. 11 shows a western blot detecting LDLR levels of liver samples from the indicated NHPs.

FIG. 12A and FIG. 12B provide liver LDLR expression levels of the indicated NHPs on day 18 post rAAV administration using ISH (FIG. 12A) and IHC (FIG. 12B).

FIG. 13A and FIG. 13B provide liver LDLR expression levels of the indicated NHPs on day 120 post rAAV administration using ISH (FIG. 13A) and IHC (FIG. 13B).

FIG. 14A and FIG. 14B provide liver LDLR expression levels of the indicated NHPs on day 18 post rAAV administration using ISH (FIG. 14A) and IHC (FIG. 14B).

FIG. 15A and FIG. 15B provide liver LDLR expression levels of the indicated NHPs on day 120 post rAAV administration using ISH (FIG. 15A) and IHC (FIG. 15B).

FIG. 16A and FIG. 16B provide liver LDLR expression levels of the indicated NHPs on day 18 post rAAV administration using ISH (FIG. 16A) and IHC (FIG. 16B).

FIG. 17A and FIG. 17B provide liver LDLR expression levels of the indicated NHPs on day 120 post rAAV administration using ISH (FIG. 17A) and IHC (FIG. 17B).

FIG. 18A and FIG. 18B provide liver LDLR expression levels of the indicated NHPs on day 18, day 83/88, and day 120 post rAAV administration using ISH (FIG. 18A) and IHC (FIG. 18B).

FIG. 19A and FIG. 19B provide a comparison of the LDLR^{-/-} Apobec^{-/-} mouse model and NHP as described, including liver serum LDL, vector copies in liver, and LDLR mRNA.

FIG. 20A and FIG. 20B show a comparison of muscle transduction and secreted protein levels in serum following intramuscular (IM) delivery of multiple capsids. Vector was delivered IM expressing mAb from muscle selected promoter or LacZ in mice. The data show that AAVrh91 achieves similar muscle transduction to AAV1 and AAV6 but has higher yields. AAV3B variants are superior to AAV8 and the parent AAV3B capsid for muscle transduction and have high yields.

### DETAILED DESCRIPTION OF THE INVENTION

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention. As used herein, the terms “a” or “an”, refers to one or more, for example, “an ocular cell” is understood to represent one or more ocular cells. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

With regard to the following description, it is intended that each of the compositions herein described, is useful, in another embodiment, in the methods of the invention. In addition, it is also intended that each of the compositions described as useful in the methods, is, in another embodiment, itself an embodiment of the invention.

Adeno-associated virus (AAV)-mediated gene therapy is a promising way to treat diseases, especially rare diseases that have very few effective treatments. AAVs isolated from natural sources have limitations in terms of gene delivery efficiency and specificity. Directed evolution has been used to generate AAV mutants that may overcome those drawbacks.

We used a scorecard approach to generate the initial diversity of the AAV3B hypervariable region (HVR) VIII. We then conducted selections in human-hepatocytes-xenografted Fah^{-/-}/Rag2^{-/-}/Il2rg^{-/-} (FRG) mice, by injecting the libraries intravenously and retrieving AAV cDNA from human hepatocytes isolated from those mice to prepare new libraries for the next rounds. Sixteen AAV3B variants that showed dramatic increase of relative frequencies were evaluated in nonhuman primates (NHPs) with a validated barcode system. Most of the 16 variants were better than AAV3B in terms of liver transduction, with some showing high liver specificity. Two variants were further evaluated with a therapeutic transgene for liver gene therapy in NHPs and the preliminary results confirmed the NHP barcode evaluation result.

A “recombinant AAV” or “rAAV” is a DNAse-resistant viral particle containing two elements, an AAV capsid and a vector genome containing at least a non-AAV coding sequence packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein. The vector genome is sometimes referred to herein as the “minigene”.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position. Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate e.g., as illustrated below.

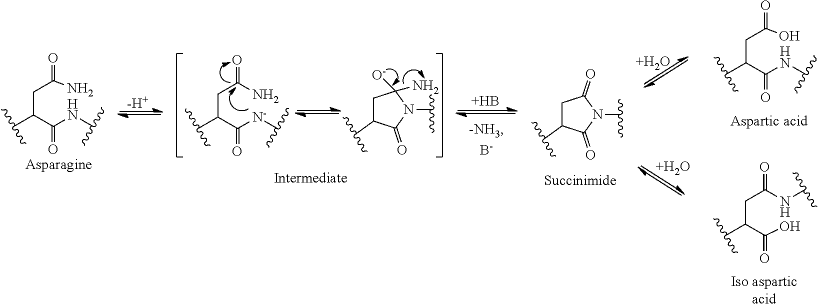

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio.

In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

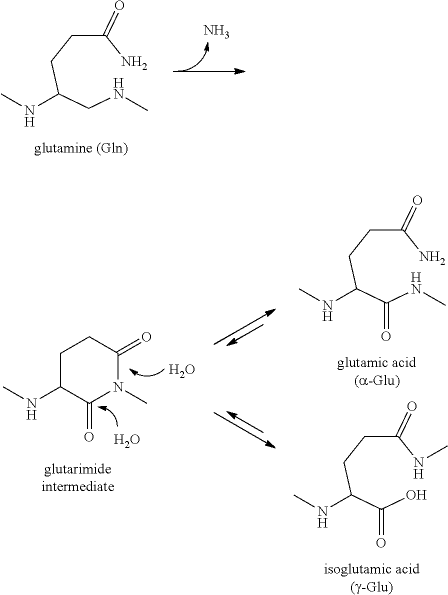

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100%, or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid.

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH₂ groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations. Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternatively one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected.

Amino acid modifications may be made by conventional genetic engineering techniques. For example, a nucleic acid sequence containing modified AAV vp codons may be generated in which one to three of the codons encoding glycine in asparagine-glycine pairs are modified to encode an amino acid other than glycine. In certain embodiments, a nucleic acid sequence containing modified asparagine codons may be engineered at one to three of the asparagine-glycine pairs, such that the modified codon encodes an amino acid other than asparagine. Each modified codon may encode a different amino acid. Alternatively, one or more of the altered codons may encode the same amino acid. In certain embodiments, these modified nucleic acid sequences may be used to generate a mutant rAAV having a capsid with lower deamidation than the native AAV3B variant capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, Calif.). One codon optimizing method is described, e.g., in International Patent Publication No. WO 2015/012924, which is incorporated by reference herein in its entirety. See also, e.g., U.S. Patent Publication No. 2014/0032186 and U.S. Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, the term “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is liver. In another embodiment, the target tissue is the heart. In another embodiment, the target tissue is brain. In another embodiment, the target tissue is muscle.

As used herein, the term “mammalian subject” or “subject” includes any mammal in need of the methods of treatment described herein or prophylaxis, including particularly humans. Other mammals in need of such treatment or prophylaxis include dogs, cats, or other domesticated animals, horses, livestock, laboratory animals, including non-human primates, etc. The subject may be male or female.

As used herein, the term “host cell” may refer to the packaging cell line in which the rAAV is produced from the plasmid. In the alternative, the term “host cell” may refer to a target cell in which expression of the transgene is desired.

### A. The AAV Capsid

Provided herein are novel AAV3B variant VP1, VP2, and VP3 capsid proteins. The full-length vp1 sequences are set forth in the sequence listing: AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.08 (SEQ ID NO: 15), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), AAV3B.AR2.16 (SEQ ID NO: 29), or AAV3B.AR2.17 (SEQ ID NO: 31).

In the examples below, AAV3B.AR2.08 and AAV3B.AR2.16 have been observed to transduce human liver (hepatocytes). AAV3B.AR2.08 and AAV3B.AR2.16 capsids are particularly well suited for generating rAAV for liver-directed gene therapy, gene editing, and other rAAV-mediated liver-targeting of gene products. However, this is not a limitation on the utility of these capsids, which may be used for targeting other tissues and organs, such as those described below.

The AAV capsid consists of three overlapping coding sequences, which vary in length due to alternative start codon usage. These variable proteins are referred to as VP1, VP2, and VP3, with VP1 being the longest and VP3 being the shortest. The AAV particle consists of all three capsid proteins at a ratio of ˜1:1:10 (VP1:VP2:VP3). VP3, which is comprised in VP1 and VP2 at the N-terminus, is the main structural component that builds the particle. The capsid protein can be referred to using several different numbering systems. For convenience, as used herein, the AAV sequences are referred to using VP1 numbering, which starts with aa 1 for the first residue of VP1. However, the capsid proteins described herein include VP1, VP2 and VP3 (used interchangeably herein with vp1, vp2 and vp3). The numbering of the variable proteins of the capsids of the invention are as follows:

### Nucleic Acids

Provided herein are AAV3B capsid variants VP1 nucleic acid sequences encoding amino acids 1 to 736; VP2 nucleic acid sequences encoding aa 138 to 736; and/or VP3 nucleic acid sequences encoding aa 203 to 736 of their respective SEQ ID NOs and using the native AAV3B as a reference (SEQ ID NO: 34). The AAV3B capsid variants are encoded by a nucleic acid of at least one of the vp1 (nt 1 to nt 2211), vp2 (nt 412 to nt 2211) and the vp3 (nt 607 to nt 2211) of their respective SEQ ID NOs and using the native AAV3B as a reference (SEQ ID NO: 33). In one embodiment, an AAV3B.AR2.08 VP1 nucleic acid sequence encodes amino acids about 1 to about 736 of SEQ ID NO: 15 (VP1) and also produces the VP2 (amino acids about 138 to about 736) and VP3 proteins (amino acids about 203 to about 736) of SEQ ID NO: 15. In certain embodiments, the AAV3B.AR2.08 VP1 nucleic acid sequence is the full-length of SEQ ID NO: 16, or a sequence at least 90% identical, at least 95%, at least 97%, at least 98%, or at least 99% identical over the consecutive nucleotide sequence of about nt 1 to about nt 2211 of SEQ ID NO: 16. In certain embodiments, the AAV3B.AR2.08 VP2 nucleic acid sequence is the full-length of SEQ ID NO: 16, or a sequence at least 90% identical, at least 95%, at least 97%, at least 98%, or at least 99% identical over the consecutive nucleotide sequence of about nt 412 to about nt 2211 of SEQ ID NO: 16. In certain embodiments, the AAV3B.AR2.08 VP3 nucleic acid sequence is the full-length of SEQ ID NO: 16, or a sequence at least 90% identical, at least 95%, at least 97%, at least 98%, or at least 99% identical over the consecutive nucleotide sequence of about nt 607 to about nt 2211 of SEQ ID NO: 16. In certain embodiments, for sequences having the recited identity to SEQ ID NO: 16, the nucleic acids in the region of nt 1744 to nt 1783 encode the amino acids at positions 582 to 594 of AAV3B.AR2.08 of SEQ ID NO: 15. In other embodiments, the sequences within the recited identity encode the full-length VP1, VP2, or VP3 of SEQ ID NO: 15.

In another embodiment, a AAV3B.AR2.16 VP1 nucleic acid sequence encodes amino acids about 1 to about 736 of SEQ ID NO: 29 (VP1) and also produced the VP2 (amino acids about 138 to about 736) and VP3 proteins (amino acids about 203 to about 736) of SEQ ID NO: 29. In certain embodiments, the AAV3B.AR2.16 VP1 nucleic acid sequence is the full-length of SEQ ID NO: 30, or a sequence at least 90% identical, at least 95%, at least 97%, at least 98%, or at least 99% identical over the consecutive nucleotide sequence of about nt 1 to about nt 2211 of SEQ ID NO: 30. In certain embodiments, the AAV3B.AR2.16 VP2 nucleic acid sequence is the full-length of SEQ ID NO: 30, or a sequence at least 90% identical, at least 95%, at least 97%, at least 98%, or at least 99% identical over the consecutive nucleotide sequence of about nt 412 to about nt 2211 of SEQ ID NO: 30. In certain embodiments, the AAV3B.AR2.16 VP3 nucleic acid sequence is the full-length of SEQ ID NO: 30, or a sequence at least 90% identical, at least 95%, at least 97%, at least 98%, or at least 99% identical over the consecutive nucleotide sequence of about nt 607 to about nt 2211 of SEQ ID NO: 30. In certain embodiments, an AAV3B.AR2.16 nucleic acid sequence encoding SEQ ID NO: 30 is provided for use in producing an AAV capsid and packing a vector genome to form a rAAV3B.AR2.16 rAAV particle. In certain embodiments, the AAV3B.AR2.16 nucleic acid sequence has the sequence of SEQ ID NO: 30 or a sequence at least 90% identical, at least 95%, at least 97%, at least 98%, or at least 99% identical thereto. In certain embodiments, for sequences having the recited identity to SEQ ID NO: 30, the nucleic acids in the region of nt 1744 to nt 1783 encode the amino acids at positions 582 to 594 of AAV3B.AR2.16 of SEQ ID NO: 30. In other embodiments, the sequences within the recited identity encode the full-length VP1, VP2 or VP3 of SEQ ID NO: 29.

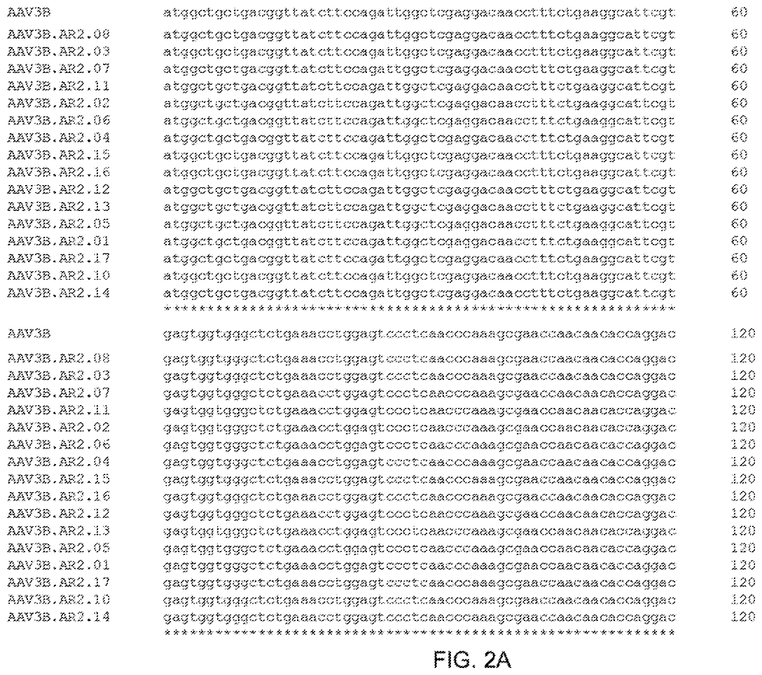

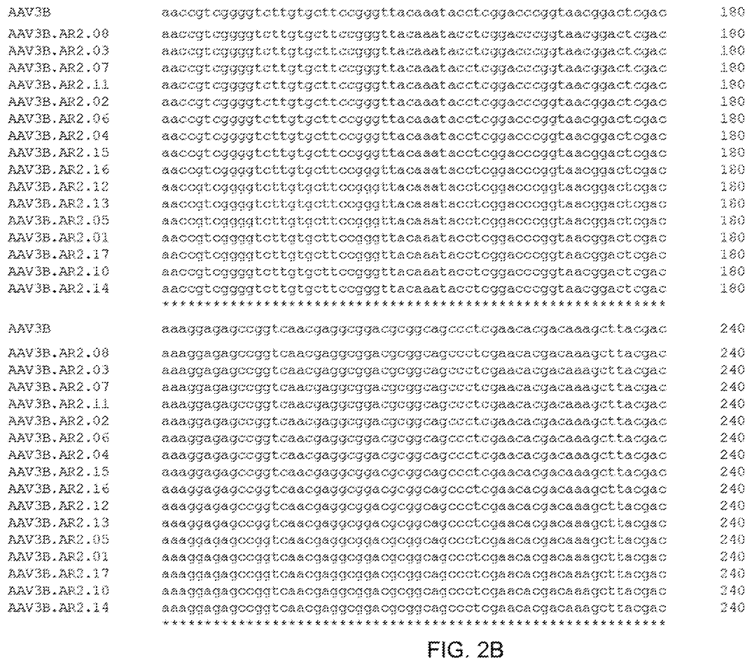

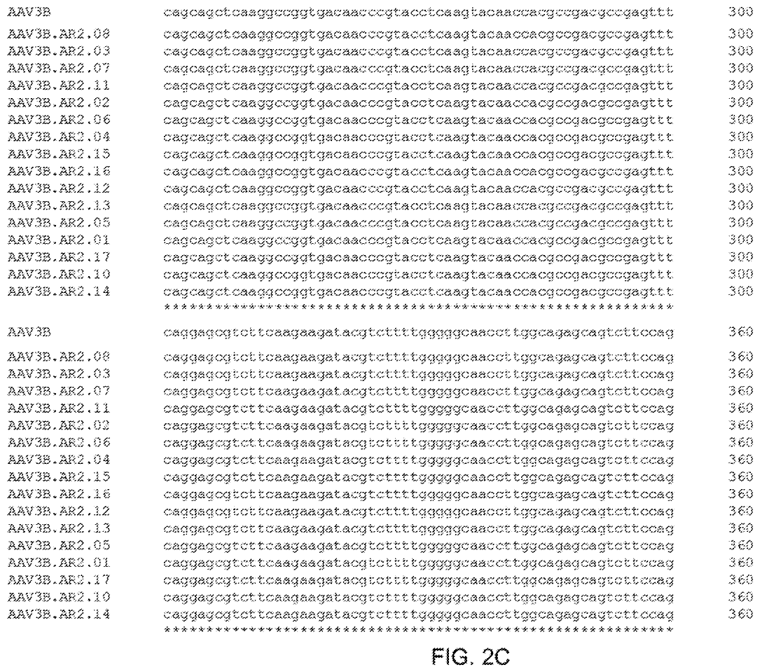

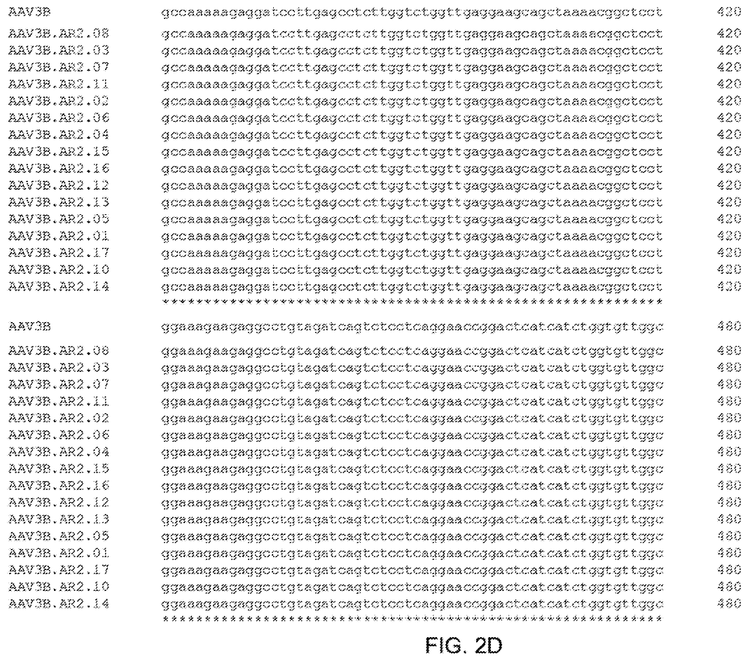

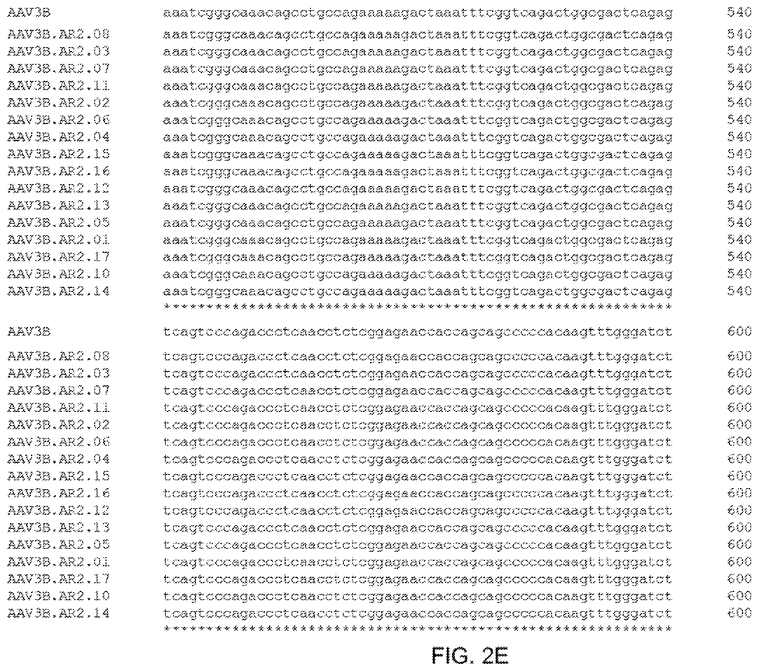

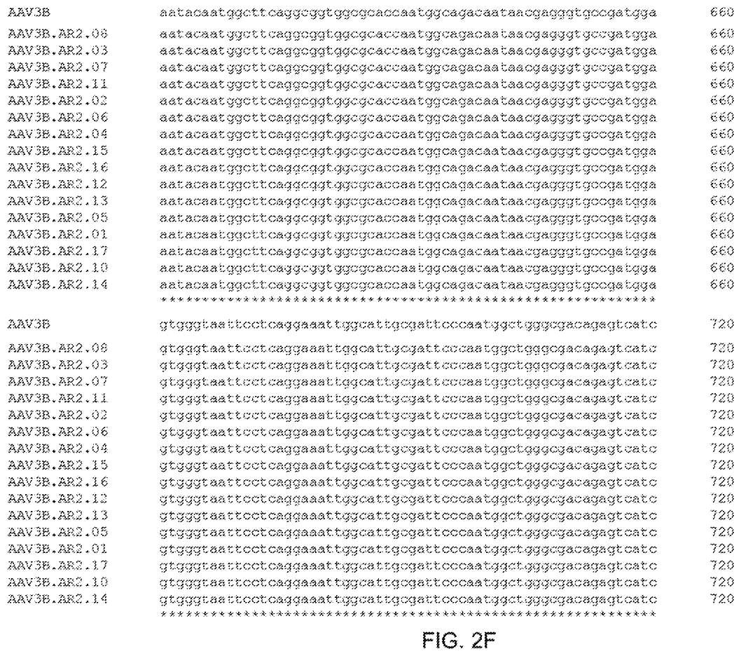

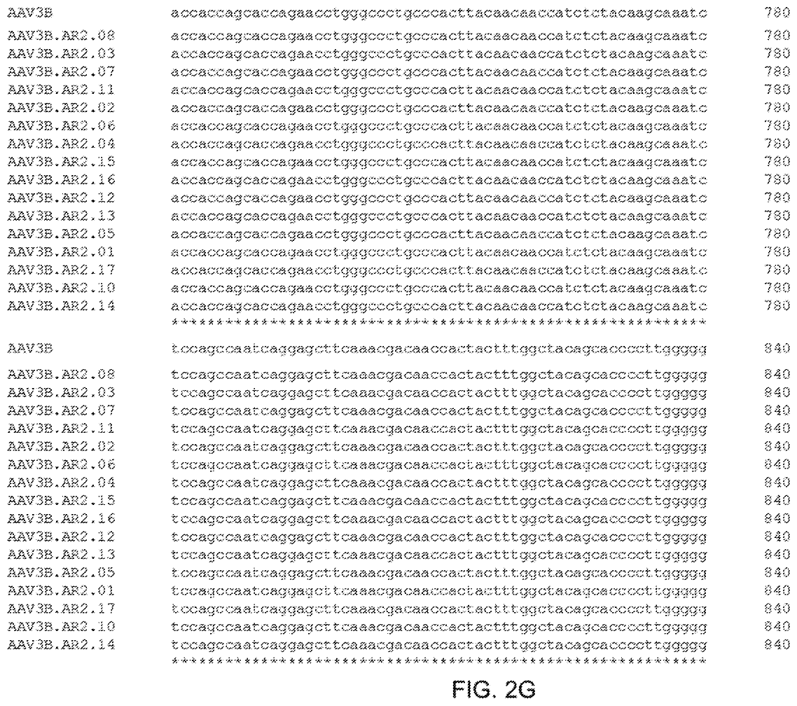

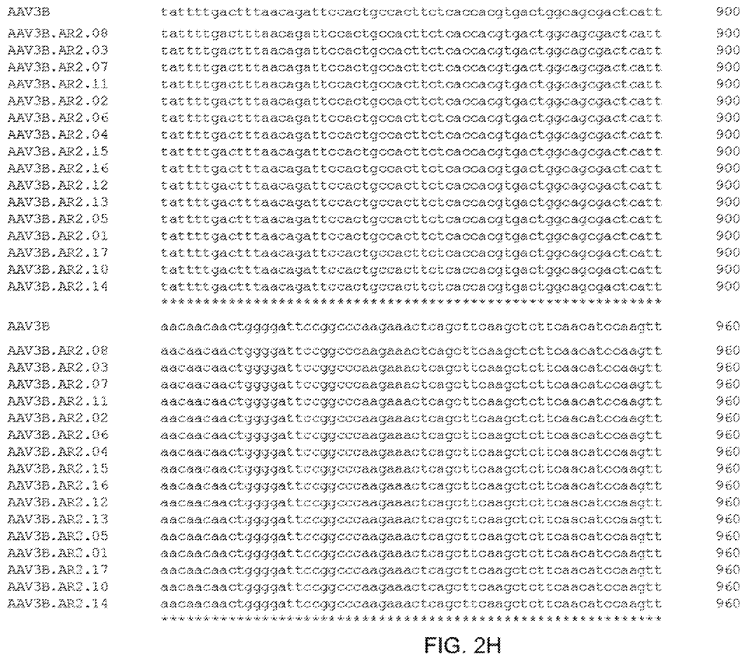

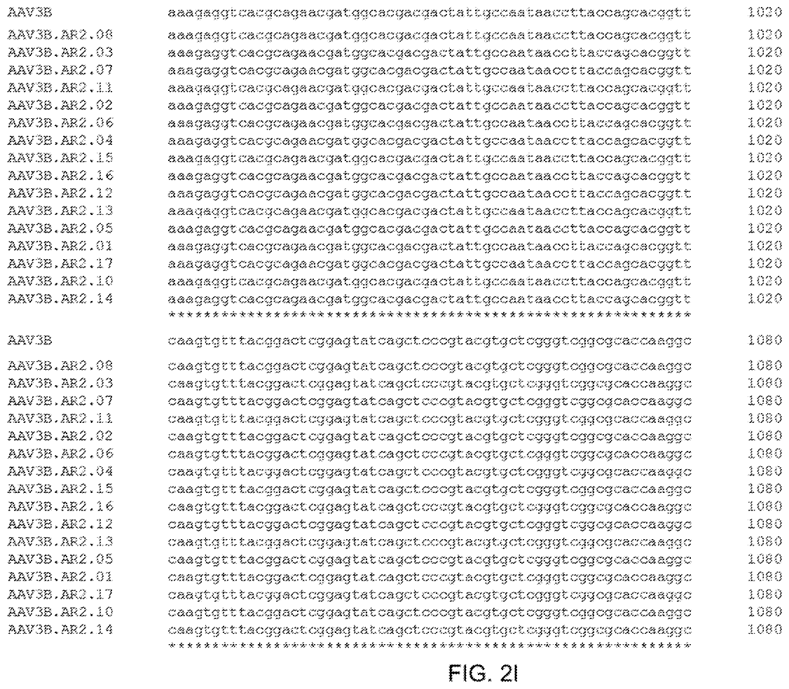

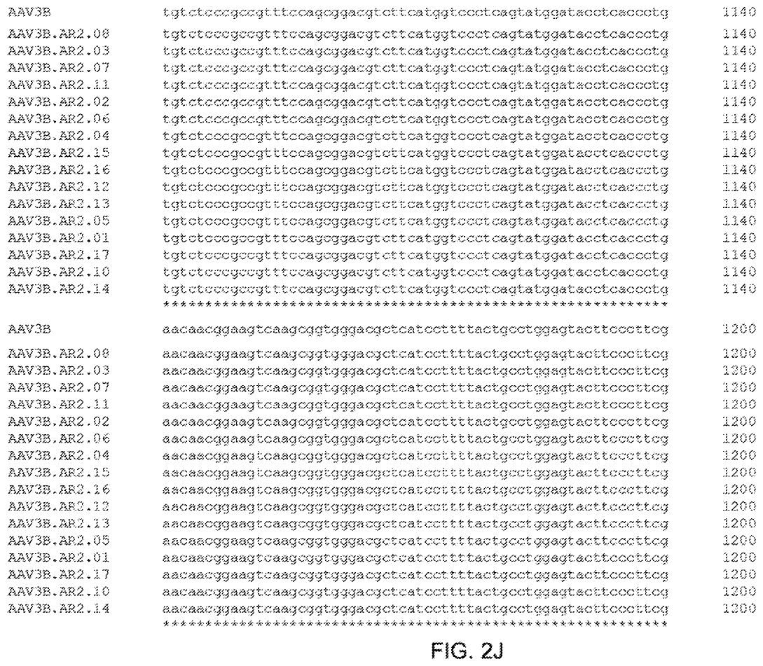

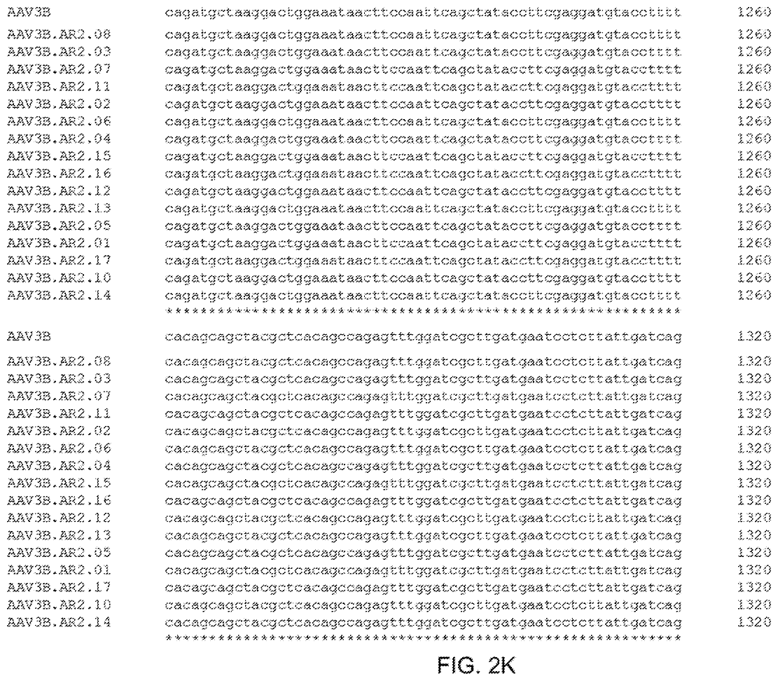

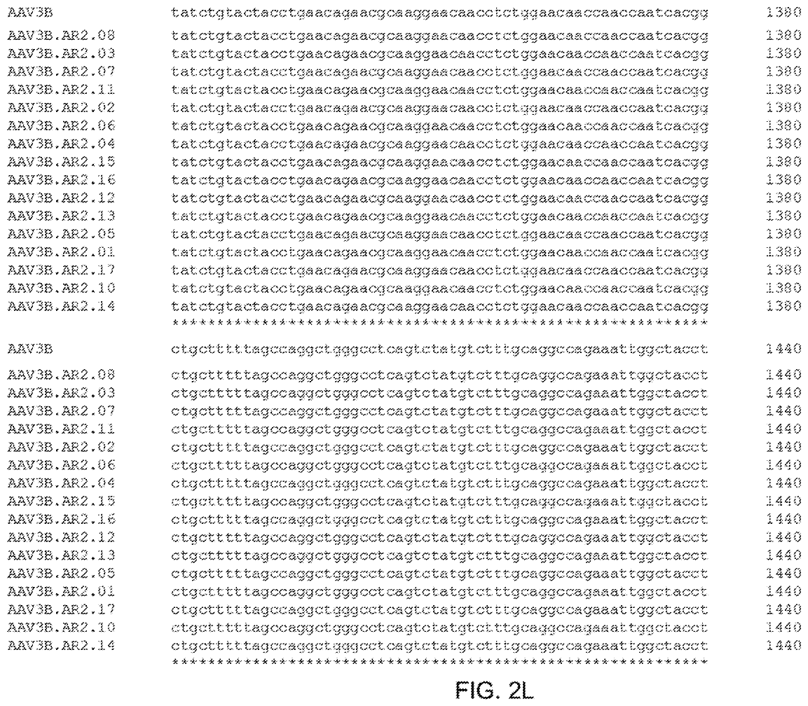

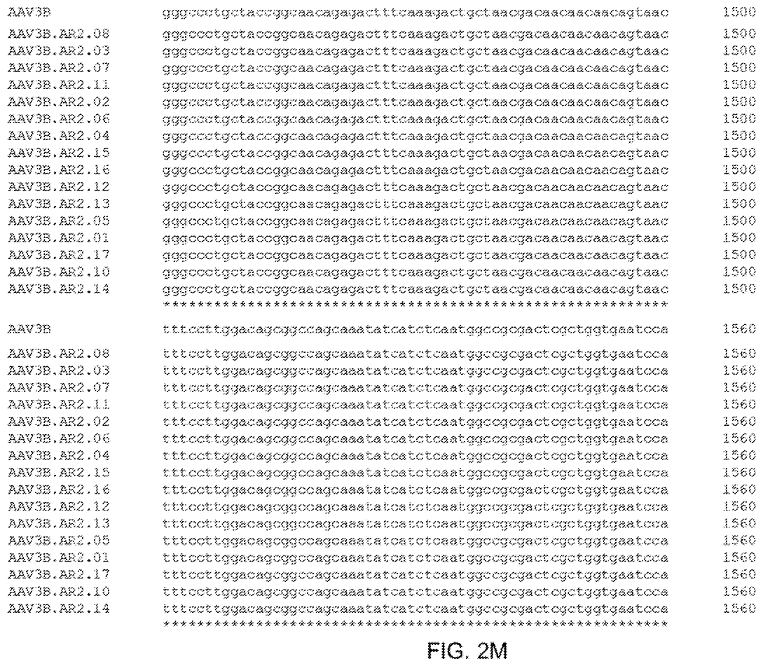

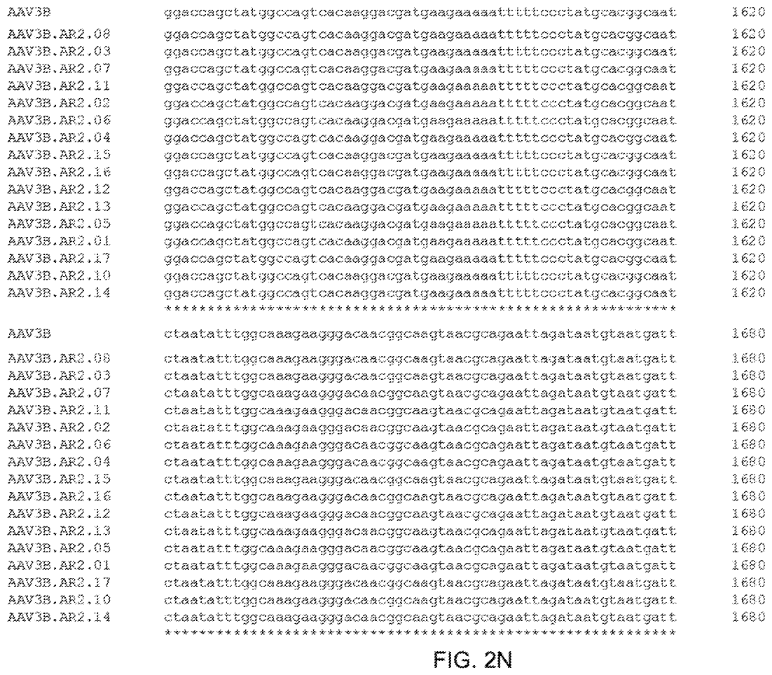

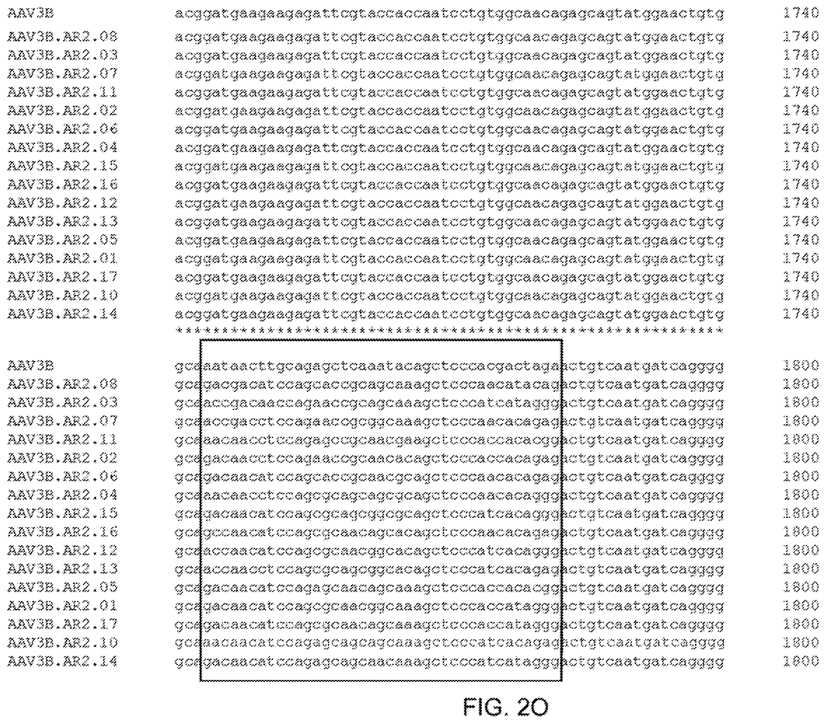

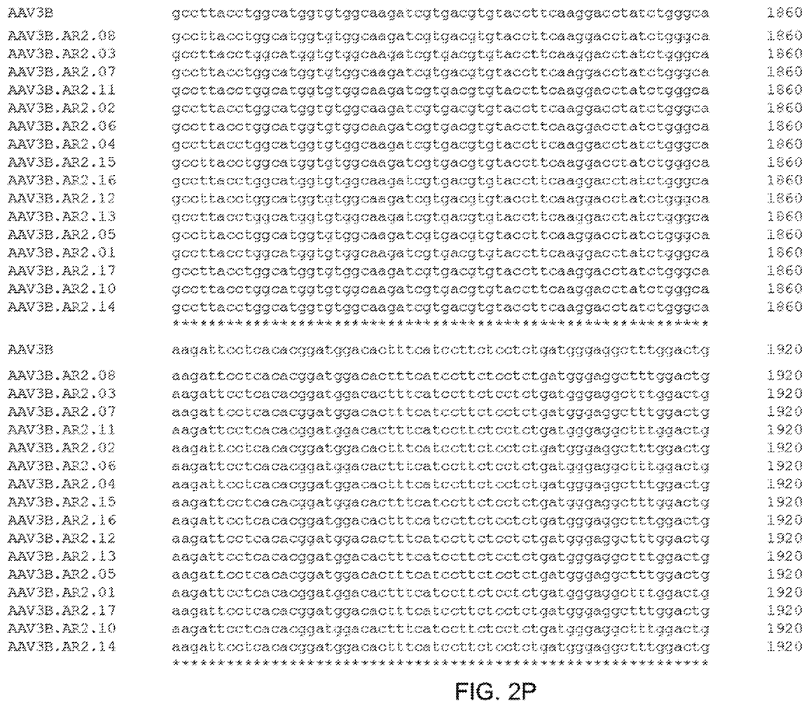

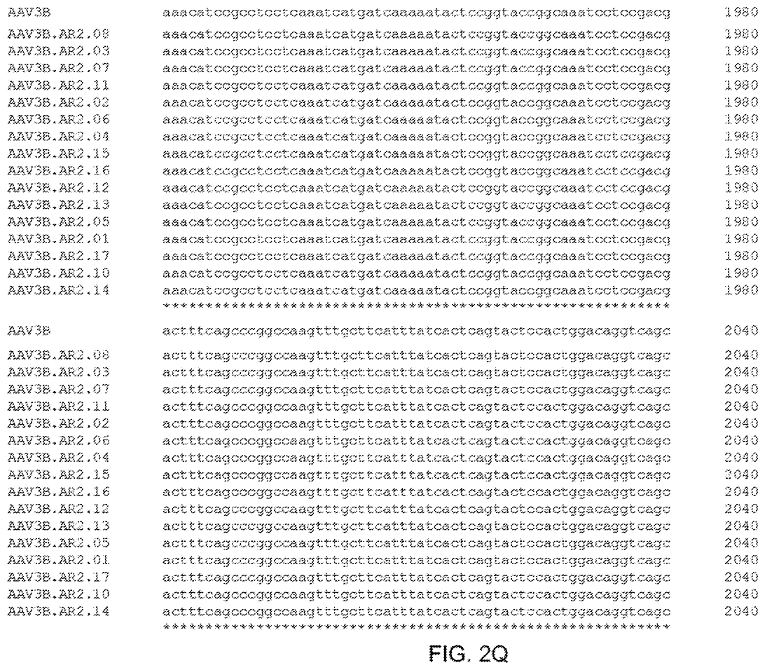

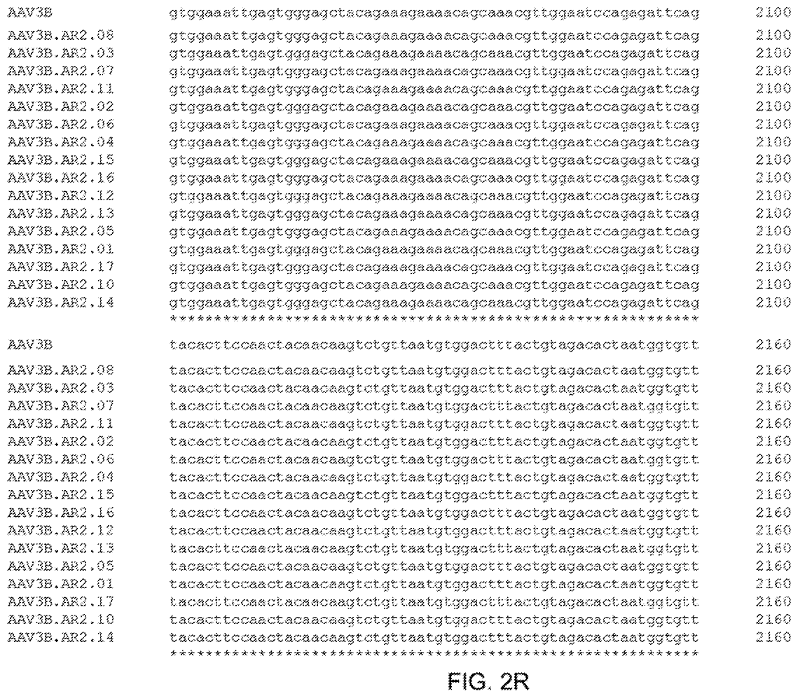

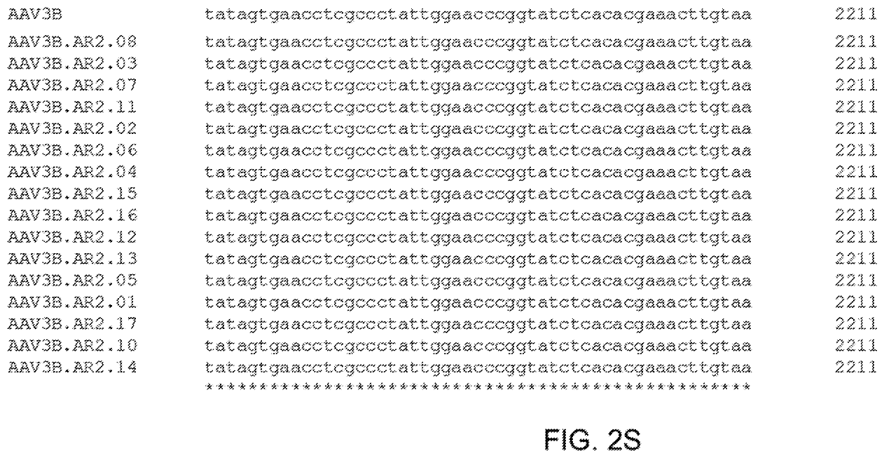

An alignment of the capsids described herein, along with AAV3B, is shown in FIG. 2A-FIG. 2S.

### Amino Acid (Aa) Sequences

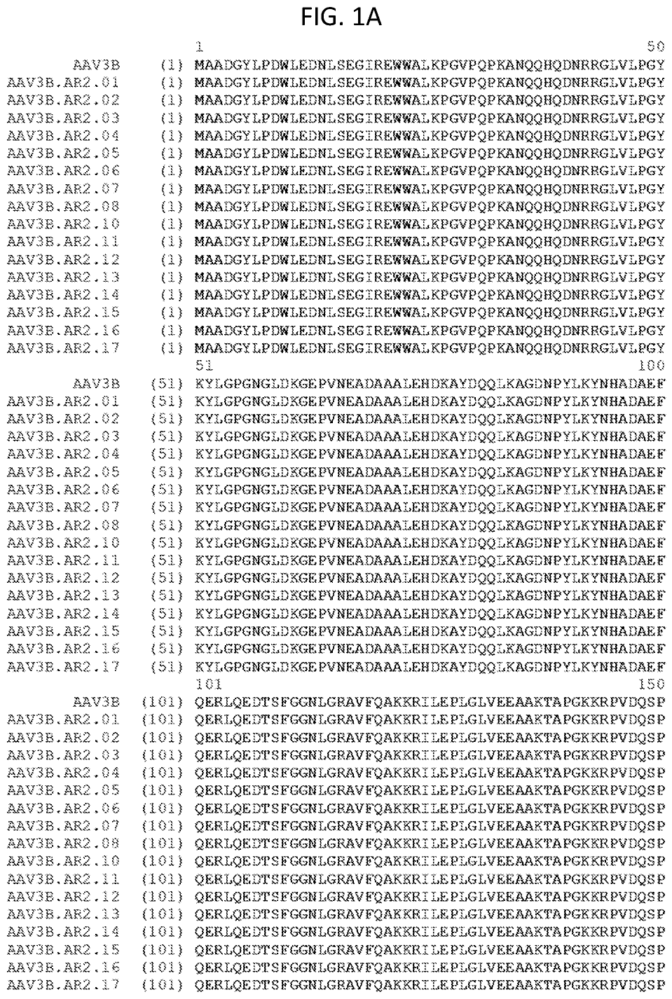

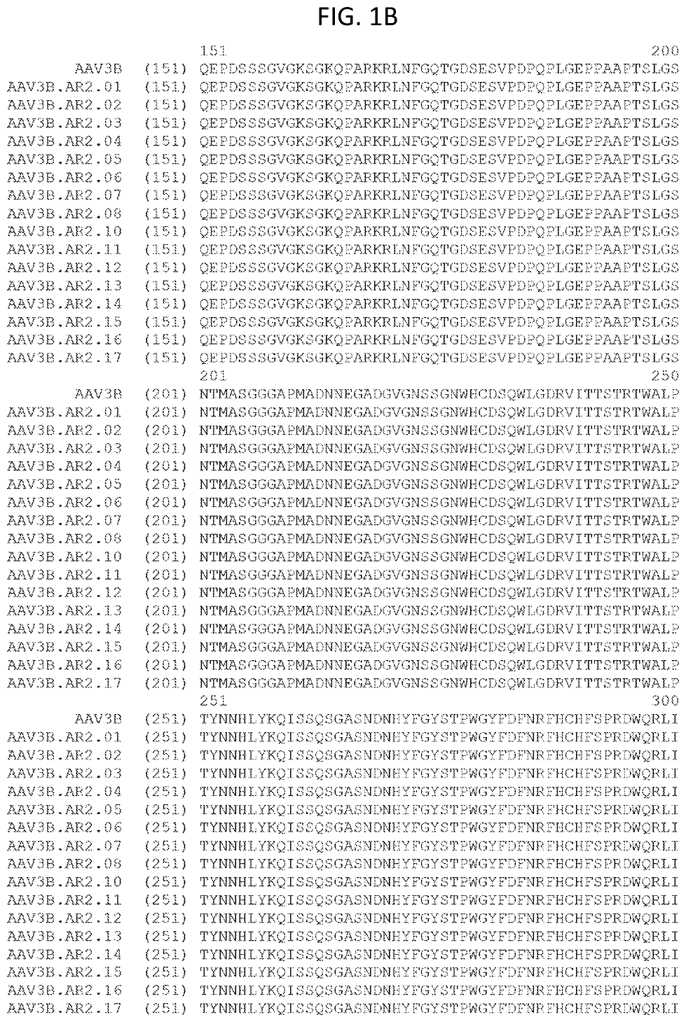

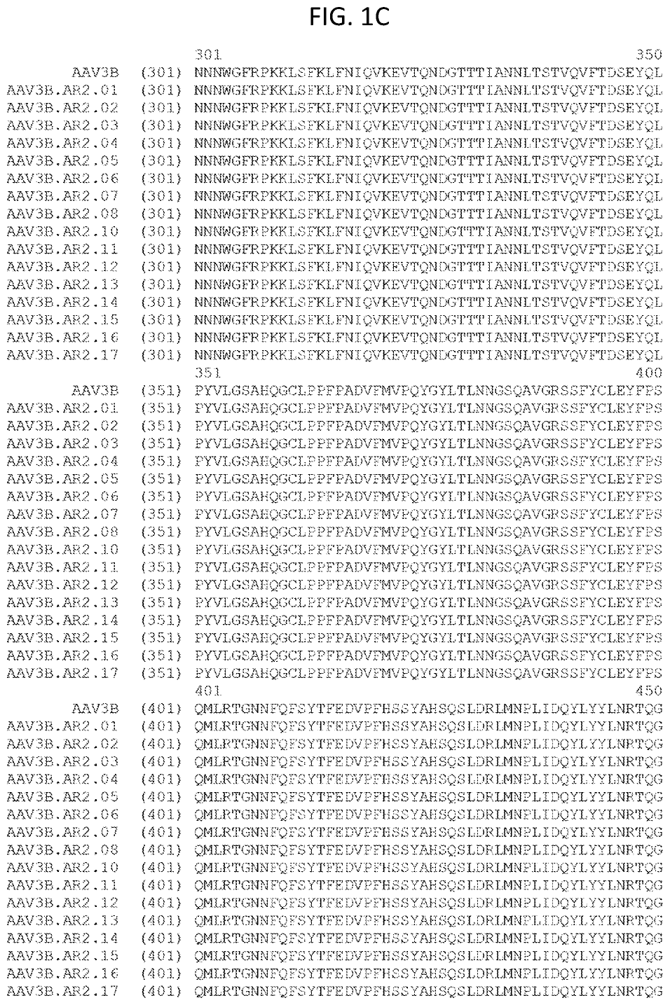

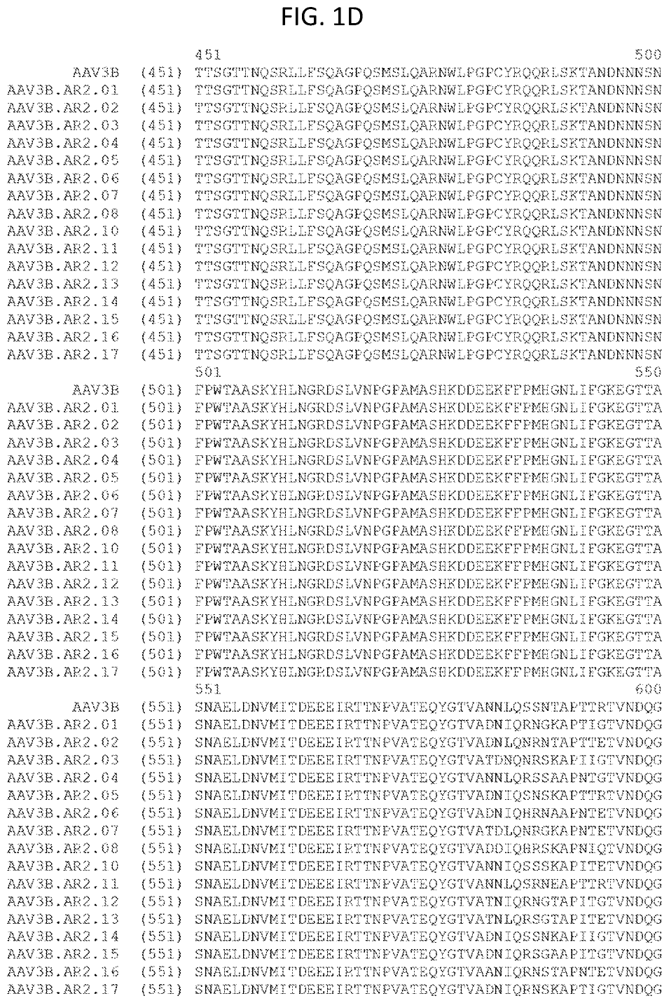

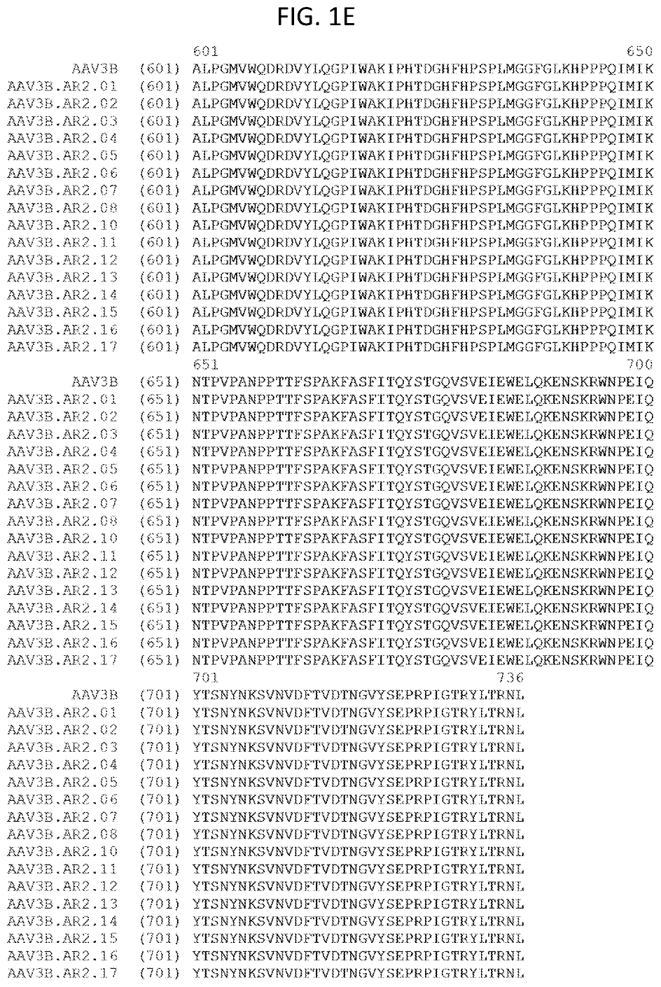

All AAV3B variants: aa vp1-1 to 736; vp2-aa 138 to 736; vp3-aa 203 to 736 of their respective SEQ ID NOs and using the native AAV3B as a reference (SEQ ID NO: 33). An alignment of the capsids described herein, along with AAV3B, is shown in FIG. 1A-FIG. 1E.

In one embodiment, the AAV3B variant capsid is produced from a nucleic acid sequence encoding the VP1 amino acid sequence of AAV3B.AR2.08 (SEQ ID NO: 15, about amino acid 1 to about amino acid 736), or a sequence having at least 95% identity, at least 97% identity, or at least 99% identical thereto in which the amino acids at positions 582 to 594 of SEQ ID NO: 15 are retained. In certain embodiments, the nucleic acid sequence encoding the VP2-specific amino acid sequence (about amino acid 138 to about amino acid 736) and/or the VP3-specific amino acid sequence (about amino acid 203 to about amino acid 736) is additionally or alternatively used in production.

In another embodiment, the AAV3B variant capsid is produced from a nucleic acid sequence encoding the VP1 amino acid sequence of AAV3B.AR2.16 (SEQ ID NO: 29), or a sequence having at least 95% identity, at least 97% identity, or at least 99% identical thereto in which the amino acids at positions 582 to 594 of SEQ ID NO: 29 are retained. In certain embodiments, the nucleic acid sequence encoding the VP2-specific amino acid sequence (about amino acid 138 to about amino acid 736) and/or the VP3-specific amino acid sequence (about amino acid 203 to about amino acid 736) is additionally or alternatively used in production.

Included herein are rAAV comprising at least one of the vp1, vp2 and the vp3 of any of AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.08 (SEQ ID NO: 15), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), AAV3B.AR2.16 (SEQ ID NO: 29), or AAV3B.AR2.17 (SEQ ID NO: 31). Also provided herein are rAAV comprising AAV capsids encoded by at least one of the vp1, vp2 and the vp3 of any of AAV3B.AR2.01 (SEQ ID NO: 2), AAV3B.AR2.02 (SEQ ID NO: 4), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.08 (SEQ ID NO: 16), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), AAV3B.AR2.16 (SEQ ID NO: 30), or AAV3B.AR2.17 (SEQ ID NO: 32).

In one embodiment, a composition is provided which includes a mixed population of recombinant adeno-associated virus (rAAV), each of said rAAV comprising: (a) an AAV capsid comprising about 60 capsid proteins made up of vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine-glycine pairs in the AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the deamidated asparagines are deamidated to aspartic acid, isoaspartic acid, an interconverting aspartic acid/isoaspartic acid pair, or combinations thereof. In certain embodiments, the capsid further comprises deamidated glutamine(s) which are deamidated to (α)-glutamic acid, γ-glutamic acid, an interconverting (α)-glutamic acid/γ-glutamic acid pair, or combinations thereof.

In certain embodiments, a novel isolated AAV3B.AR2.01 capsid is provided. The nucleic acid sequence encoding the AAV3B.AR2.01 capsid is provided in SEQ ID NO: 2 and the predicted encoded amino acid sequence is provided in SEQ ID NO: 1. Provided herein is an rAAV comprising at least one of the vp1 (aa 1 to 736), vp2 (aa 138 to 736) and the vp3 (aa 203 to 736) of AAV3B.AR2.01 (SEQ ID NO: 1). Also provided herein are rAAV comprising an AAV capsid encoded by at least one of the vp1 (nt 1 to nt 2211), vp2 (nt 412 to nt 2211) and the vp3 (nt 607 to nt 2211) sequences of AAV3B.AR2.01 (SEQ ID NO: 2).

In a further aspect, a recombinant adeno-associated virus (rAAV) is provided which comprises: (A) an AAV3B.AR2.01 capsid comprising one or more of: (1) AAV3B.AR2.01 capsid proteins comprising: a heterogeneous population of AAV3B.AR2.01 vp1 proteins selected from: vp1 proteins produced by expression from a nucleic acid sequence which encodes the predicted amino acid sequence of 1 to 736 of SEQ ID NO: 1, vp1 proteins produced from SEQ ID NO: 2, or vp1 proteins produced from a nucleic acid sequence at least 70% identical to SEQ ID NO: 2 which encodes the predicted amino acid sequence of 1 to 736 of SEQ ID NO: 1, a heterogeneous population of AAV3B.AR2.01 vp2 proteins selected from: vp2 proteins produced by expression from a nucleic acid sequence which encodes the predicted amino acid sequence of at least about amino acids 138 to 736 of SEQ ID NO: 1, vp2 proteins produced from a sequence comprising at least nucleotides 412 to 2211 of SEQ ID NO: 2, or vp2 proteins produced from a nucleic acid sequence at least 70% identical to at least nucleotides 412 to 2211 of SEQ ID NO: 2 which encodes the predicted amino acid sequence of at least about amino acids 138 to 736 of SEQ ID NO: 1, a heterogeneous population of AAV3B.AR2.01 vp3 proteins selected from: vp3 proteins produced by expression from a nucleic acid sequence which encodes the predicted amino acid sequence of at least about amino acids 203 to 736 of SEQ ID NO: 1, vp3 proteins produced from a sequence comprising at least nucleotides 607 to 2211 of SEQ ID NO: 2, or vp3 proteins produced from a nucleic acid sequence at least 70% identical to at least nucleotides 607 to 2211 of SEQ ID NO: 2 which encodes the predicted amino acid sequence of at least about amino acids 203 to 736 of SEQ ID NO: 1; and/or (2) a heterogeneous population of vp1 proteins which are the product of a nucleic acid sequence encoding the amino acid sequence of SEQ ID NO: 1, a heterogeneous population of vp2 proteins which are the product of a nucleic acid sequence encoding the amino acid sequence of at least about amino acids 138 to 736 of SEQ ID NO: 1, and a heterogeneous population of vp3 proteins which are the product of a nucleic acid sequence encoding at least amino acids 203 to 736 of SEQ ID NO: 1, wherein: the vp1, vp2 and vp3 proteins contain subpopulations with amino acid modifications comprising at least two highly deamidated asparagines (N) in asparagine-glycine pairs in SEQ ID NO: 1 and optionally further comprising subpopulations comprising other deamidated amino acids, wherein the deamidation results in an amino acid change; and (B) a vector genome in the AAV3B.AR2.01 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.01 capsid comprises: a heterogeneous population of vp1 proteins which are the product of a nucleic acid sequence encoding the amino acid sequence of SEQ ID NO: 1, a heterogeneous population of vp2 proteins which are the product of a nucleic acid sequence encoding the amino acid sequence of at least about amino acids 138 to 736 of SEQ ID NO: 1, and a heterogeneous population of vp3 proteins which are the product of a nucleic acid sequence encoding at least amino acids 203 to 736 of SEQ ID NO: 1.

In certain embodiments, the nucleic acid sequence encoding the AAV3B.AR2.01 vp1 capsid protein is provided in SEQ ID NO: 2. In other embodiments, a nucleic acid sequence of 70% to 99.9% identity to SEQ ID NO: 2 may be selected to express the AAV3B.AR2.01 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 2. However, other nucleic acid sequences which encode the amino acid sequence of SEQ ID NO: 1 may be selected for use in producing rAAV capsids. In certain embodiments, the nucleic acid sequence has the nucleic acid sequence of SEQ ID NO: 2 or a sequence at least 70% to 99% identical, at least 75%, at least 80%, at least 85%, at least 90%, at least 95%, at least 97%, at least 99%, identical to SEQ ID NO: 2 which encodes SEQ ID NO: 1. In certain embodiments, the nucleic acid sequence has the nucleic acid sequence of SEQ ID NO: 2 or a sequence at least 70% to 99%, at least 75%, at least 80%, at least 85%, at least 90%, at least 95%, at least 97%, at least 99%, identical to about nt 412 to about nt 2211 of SEQ ID NO: 2 which encodes the vp2 capsid protein (about aa 138 to 736) of SEQ ID NO: 1. In certain embodiments, the nucleic acid sequence has the nucleic acid sequence of about nt 607 to about nt 2211 of SEQ ID NO: 2 or a sequence at least 70% to 99%, at least 75%, at least 80%, at least 85%, at least 90%, at least 95%, at least 97%, at least 99%, identical to nt SEQ ID NO: 2 which encodes the vp3 capsid protein (about aa 203 to 736) of SEQ ID NO: 1.

The invention also encompasses nucleic acid sequences encoding mutant AAV3B.AR2.01, in which one or more residues has been altered in order to decrease deamidation, or other modifications which are identified herein. Such nucleic acid sequences can be used in production of mutant AAV3B.AR2.01 capsids.

In certain embodiments, a novel isolated AAV3B.AR2.02 capsid is provided. The nucleic acid sequence encoding the AAV is provided in SEQ ID NO: 4 and the predicted encoded amino acid sequence is provided in SEQ ID NO: 3. Provided herein is an rAAV comprising at least one of the vp1 (aa 1 to 736), vp2 (aa 138 to 736), and the vp3 (aa 203 to 736) of AAV3B.AR2.02 (SEQ ID NO: 3). Also provided herein are rAAV comprising an AAV capsid encoded by at least one of the vp1 (nt 1 to nt 2211), vp2 (nt 412 to nt 2211), and the vp3 (nt 607 to nt 2211) AAV3B.AR2.02 (SEQ ID NO: 4).

In a further aspect, a recombinant adeno-associated virus (rAAV) is provided which comprises: (A) an AAV3B.AR2.02 capsid comprising one or more of: (1) AAV3B.AR2.02 capsid proteins comprising: a heterogeneous population of AAV3B.AR2.02 vp1 proteins selected from: vp1 proteins produced by expression from a nucleic acid sequence which encodes the predicted amino acid sequence of 1 to 736 of SEQ ID NO: 3, vp1 proteins produced from SEQ ID NO: 4, or vp1 proteins produced from a nucleic acid sequence at least 70% identical to SEQ ID NO: 4 which encodes the predicted amino acid sequence of 1 to 736 of SEQ ID NO: 3, a heterogeneous population of AAV3B.AR2.02 vp2 proteins selected from: vp2 proteins produced by expression from a nucleic acid sequence which encodes the predicted amino acid sequence of at least about amino acids 138 to 736 of SEQ ID NO: 3, vp2 proteins produced from a sequence comprising at least nucleotides 412 to 2211 of SEQ ID NO: 4, or vp2 proteins produced from a nucleic acid sequence at least 70% identical to at least nucleotides 412 to 2211 of SEQ ID NO: 4 which encodes the predicted amino acid sequence of at least about amino acids 138 to 736 of SEQ ID NO: 3, a heterogeneous population of AAV3B.AR2.02 vp3 proteins selected from: vp3 proteins produced by expression from a nucleic acid sequence which encodes the predicted amino acid sequence of at least about amino acids 203 to 736 of SEQ ID NO: 3, vp3 proteins produced from a sequence comprising at least nucleotides 607 to 2211 of SEQ ID NO: 4, or vp3 proteins produced from a nucleic acid sequence at least 70% identical to at least nucleotides 607 to 2211 of SEQ ID NO: 4 which encodes the predicted amino acid sequence of at least about amino acids 203 to 736 of SEQ ID NO: 3; and/or (2) a heterogeneous population of vp1 proteins which are the product of a nucleic acid sequence encoding the amino acid sequence of SEQ ID NO: 3, a heterogeneous population of vp2 proteins which are the product of a nucleic acid sequence encoding the amino acid sequence of at least about amino acids 138 to 736 of SEQ ID NO: 3, and a heterogeneous population of vp3 proteins which are the product of a nucleic acid sequence encoding at least amino acids 203 to 736 of SEQ ID NO: 3, wherein: the vp1, vp2 and vp3 proteins contain subpopulations with amino acid modifications comprising at least two highly deamidated asparagines (N) in asparagine-glycine pairs in SEQ ID NO: 3 and optionally further comprising subpopulations comprising other deamidated amino acids, wherein the deamidation results in an amino acid change; and (B) a vector genome in the AAV3B.AR2.02 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.02 capsid comprises: a heterogeneous population of vp1 proteins which are the product of a nucleic acid sequence encoding the amino acid sequence of SEQ ID NO: 3, a heterogeneous population of vp2 proteins which are the product of a nucleic acid sequence encoding the amino acid sequence of at least about amino acids 138 to 736 of SEQ ID NO: 3, and a heterogeneous population of vp3 proteins which are the product of a nucleic acid sequence encoding at least amino acids 203 to 736 of SEQ ID NO: 3.

In certain embodiments, the nucleic acid sequence encoding the AAV3B.AR2.02 vp1 capsid protein is provided in SEQ ID NO: 4. In other embodiments, a nucleic acid sequence of 70% to 99.9% identity to SEQ ID NO: 4 may be selected to express the AAV3B.AR2.02 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 4. However, other nucleic acid sequences which encode the amino acid sequence of SEQ ID NO: 3 may be selected for use in producing rAAV capsids. In certain embodiments, the nucleic acid sequence has the nucleic acid sequence of SEQ ID NO: 4 or a sequence at least 70% to 99% identical, at least 75%, at least 80%, at least 85%, at least 90%, at least 95%, at least 97%, at least 99%, identical to SEQ ID NO: 4 which encodes SEQ ID NO: 3. In certain embodiments, the nucleic acid sequence has the nucleic acid sequence of SEQ ID NO: 4 or a sequence at least 70% to 99%, at least 75%, at least 80%, at least 85%, at least 90%, at least 95%, at least 97%, at least 99%, identical to about nt 412 to about nt 2211 of SEQ ID NO: 4 which encodes the vp2 capsid protein (about aa 138 to 736) of SEQ ID NO: 3. In certain embodiments, the nucleic acid sequence has the nucleic acid sequence of about nt 607 to about nt 2211 of SEQ ID NO: 4 or a sequence at least 70% to 99%, at least 75%, at least 80%, at least 85%, at least 90%, at least 95%, at least 97%, at least 99%, identical to nt SEQ ID NO: 4 which encodes the vp3 capsid protein (about aa 203 to 736) of SEQ ID NO: 3.

The invention also encompasses nucleic acid sequences encoding mutant AAV3B.AR2.02, in which one or more residues has been altered in order to decrease deamidation, or other modifications which are identified herein. Such nucleic acid sequences can be used in production of mutant AAV3B.AR2.02 capsids.

In certain embodiments, a novel isolated AAV3B.AR2.03 capsid is provided. The nucleic acid sequence encoding the AAV is provided in SEQ ID NO: 6 and the predicted encoded amino acid sequence is provided in SEQ ID NO: 5. Provided herein is an rAAV comprising at least one of the vp1 (aa 1 to 736), vp2 (aa 138 to 736) and the vp3 (aa 203 to 736) of AAV3B.AR2.03 (SEQ ID NO: 5). Also provided herein are rAAV comprising an AAV capsid encoded by at least one of the vp1 (nt 1 to nt 2211), vp2 (nt 412 to nt 2211) and the vp3 (nt 607 to nt 2211) AAV3B.AR2.03 (SEQ ID NO: 6).

In a further aspect, a recombinant adeno-associated virus (rAAV) is provided which comprises: (A) an AAV3B.AR2.03 capsid comprising one or more of: (1) AAV3B.AR2.03 capsid proteins comprising: a heterogeneous population of AAV3B.AR2.03 vp1 proteins selected from: vp1 proteins produced by expression from a nucleic acid sequence which encodes the predicted amino acid sequence of 1 to 736 of SEQ ID NO: 5, vp1 proteins produced from SEQ ID NO: 6, or vp1 proteins produced from a nucleic acid sequence at least 70% identical to SEQ ID NO: 6 which encodes the predicted amino acid sequence of 1 to 736 of SEQ ID NO: 5, a heterogeneous population of AAV3B.AR2.03 vp2 proteins selected from: vp2 proteins produced by expression from a nucleic acid sequence which encodes the predicted amino acid sequence of at least about amino acids 138 to 736 of SEQ ID NO: 5, vp2 proteins produced from a sequence comprising at least nucleotides 412 to 2211 of SEQ ID NO: 6, or vp2 proteins produced from a nucleic acid sequence at least 70% identical to at least nucleotides 412 to 2211 of SEQ ID NO: 6 which encodes the predicted amino acid sequence of at least about amino acids 138 to 736 of SEQ ID NO: 5, a heterogeneous population of AAV3B.AR2.03 vp3 proteins selected from: vp3 proteins produced by expression from a nucleic acid sequence which encodes the predicted amino acid sequence of at least about amino acids 203 to 736 of SEQ ID NO: 5, vp3 proteins produced from a sequence comprising at least nucleotides 607 to 2211 of SEQ ID NO: 6, or vp3 proteins produced from a nucleic acid sequence at least 70% identical to at least nucleotides 607 to 2211 of SEQ ID NO: 6 which encodes the predicted amino acid sequence of at least about amino acids 203 to 736 of SEQ ID NO: 5; and/or (2) a heterogeneous population of vp1 proteins which are the product of a nucleic acid sequence encoding the amino acid sequence of SEQ ID NO: 5, a heterogeneous population of vp2 proteins which are the product of a nucleic acid sequence encoding the amino acid sequence of at least about amino acids 138 to 736 of SEQ ID NO: 5, and a heterogeneous population of vp3 proteins which are the product of a nucleic acid sequence encoding at least amino acids 203 to 736 of SEQ ID NO: 5, wherein: the vp1, vp2 and vp3 proteins contain subpopulations with amino acid modifications comprising at least two highly deamidated asparagines (N) in asparagine-glycine pairs in SEQ ID NO: 5 and optionally further comprising subpopulations comprising other deamidated amino acids, wherein the deamidation results in an amino acid change; and (B) a vector genome in the AAV3B.AR2.03 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.03 capsid comprises: a heterogeneous population of vp1 proteins which are the product of a nucleic acid sequence encoding the amino acid sequence of SEQ ID NO: 5, a heterogeneous population of vp2 proteins which are the product of a nucleic acid sequence encoding the amino acid sequence of at least about amino acids 138 to 736 of SEQ ID NO: 5, and a heterogeneous population of vp3 proteins which are the product of a nucleic acid sequence encoding at least amino acids 203 to 736 of SEQ ID NO: 5.

In certain embodiments, the nucleic acid sequence encoding the AAV3B.AR2.03 vp1 capsid protein is provided in SEQ ID NO: 6. In other embodiments, a nucleic acid sequence of 70% to 99.9% identity to SEQ ID NO: 6 may be selected to express the AAV3B.AR2.03 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 6. However, other nucleic acid sequences which encode the amino acid sequence of SEQ ID NO: 5 may be selected for use in producing rAAV capsids. In certain embodiments, the nucleic acid sequence has the nucleic acid sequence of SEQ ID NO: 6 or a sequence at least 70% to 99% identical, at least 75%, at least 80%, at least 85%, at least 90%, at least 95%, at least 97%, at least 99%, identical to SEQ ID NO: 6 which encodes SEQ ID NO: 5. In certain embodiments, the nucleic acid sequence has the nucleic acid sequence of SEQ ID NO: 6 or a sequence at least 70% to 99%, at least 75%, at least 80%, at least 85%, at least 90%, at least 95%, at least 97%, at least 99%, identical to about nt 412 to about nt 2211 of SEQ ID NO: 6 which encodes the vp2 capsid protein (about aa 138 to 736) of SEQ ID NO: 5. In certain embodiments, the nucleic acid sequence has the nucleic acid sequence of about nt 607 to about nt 2211 of SEQ ID NO: 6 or a sequence at least 70% to 99%, at least 75%, at least 80%, at least 85%, at least 90%, at least 95%, at least 97%, at least 99%, identical to nt SEQ ID NO: 6 which encodes the vp3 capsid protein (about aa 203 to 736) of SEQ ID NO: 5.

The invention also encompasses nucleic acid sequences encoding mutant AAV3B.AR2.03, in which one or more residues has been altered in order to decrease deamidation, or other modifications which are identified herein. Such nucleic acid sequences can be used in production of mutant AAV3B.AR2.03 capsids.

In certain embodiments, a novel isolated AAV3B.AR2.04 capsid is provided. The nucleic acid sequence encoding the AAV is provided in SEQ ID NO: 8 and the predicted encoded amino acid sequence is provided in SEQ ID NO: 7. Provided herein is an rAAV comprising at least one of the vp1 (aa 1 to 736), vp2 (aa 138 to 736) and the vp3 (aa 203 to 736) of AAV3B.AR2.04 (SEQ ID NO: 7). Also provided herein are rAAV comprising an AAV capsid encoded by at least one of the vp1 (nt 1 to nt 2211), vp2 (nt 412 to nt 2211) and the vp3 (nt 607 to nt 2211) AAV3B.AR2.04 (SEQ ID NO: 8).

In a further aspect, a recombinant adeno-associated virus (rAAV) is provided which comprises: (A) an AAV3B.AR2.04 capsid comprising one or more of: (1) AAV3B.AR2.04 capsid proteins comprising: a heterogeneous population of AAV3B.AR2.04 vp1 proteins selected from: vp1 proteins produced by expression from a nucleic acid sequence which encodes the predicted amino acid sequence of 1 to 736 of SEQ ID NO: 7, vp1 proteins produced from SEQ ID NO: 8, or vp1 proteins produced from a nucleic acid sequence at least 70% identical to SEQ ID NO: 8 which encodes the predicted amino acid sequence of 1 to 736 of SEQ ID NO: 7, a heterogeneous population of AAV3B.AR2.04 vp2 proteins selected from: vp2 proteins produced by expression from a nucleic acid sequence which encodes the predicted amino acid sequence of at least about amino acids 138 to 736 of SEQ ID NO: 7, vp2 proteins produced from a sequence comprising at least nucleotides 412 to 2211 of SEQ ID NO: 8, or vp2 proteins produced from a nucleic acid sequence at least 70% identical to at least nucleotides 412 to 2211 of SEQ ID NO: 8 which encodes the predicted amino acid sequence of at least about amino acids 138 to 736 of SEQ ID NO: 7, a heterogeneous population of AAV3B.AR2.04 vp3 proteins selected from: vp3 proteins produced by expression from a nucleic acid sequence which encodes the predicted amino acid sequence of at least about amino acids 203 to 736 of SEQ ID NO: 7, vp3 proteins produced from a sequence comprising at least nucleotides 607 to 2211 of SEQ ID NO: 8, or vp3 proteins produced from a nucleic acid sequence at least 70% identical to at least nucleotides 607 to 2211 of SEQ ID NO: 8 which encodes the predicted amino acid sequence of at least about amino acids 203 to 736 of SEQ ID NO: 7; and/or (2) a heterogeneous population of vp1 proteins which are the product of a nucleic acid sequence encoding the amino acid sequence of SEQ ID NO: 7, a heterogeneous population of vp2 proteins which are the product of a nucleic acid sequence encoding the amino acid sequence of at least about amino acids 138 to 736 of SEQ ID NO: 7, and a heterogeneous population of vp3 proteins which are the product of a nucleic acid sequence encoding at least amino acids 203 to 736 of SEQ ID NO: 7, wherein: the vp1, vp2 and vp3 proteins contain subpopulations with amino acid modifications comprising at least two highly deamidated asparagines (N) in asparagine-glycine pairs in SEQ ID NO: 7 and optionally further comprising subpopulations comprising other deamidated amino acids, wherein the deamidation results in an amino acid change; and (B) a vector genome in the AAV3B.AR2.04 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.04 capsid comprises: a heterogeneous population of vp1 proteins which are the product of a nucleic acid sequence encoding the amino acid sequence of SEQ ID NO: 7, a heterogeneous population of vp2 proteins which are the product of a nucleic acid sequence encoding the amino acid sequence of at least about amino acids 138 to 736 of SEQ ID NO: 7, and a heterogeneous population of vp3 proteins which are the product of a nucleic acid sequence encoding at least amino acids 203 to 736 of SEQ ID NO: 7.

In certain embodiments, the nucleic acid sequence encoding the AAV3B.AR2.04 vp1 capsid protein is provided in SEQ ID NO: 8. In other embodiments, a nucleic acid sequence of 70% to 99.9% identity to SEQ ID NO: 8 may be selected to express the AAV3B.AR2.04 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 8. However, other nucleic acid sequences which encode the amino acid sequence of SEQ ID NO: 7 may be selected for use in producing rAAV capsids. In certain embodiments, the nucleic acid sequence has the nucleic acid sequence of SEQ ID NO: 8 or a sequence at least 70% to 99% identical, at least 75%, at least 80%, at least 85%, at least 90%, at least 95%, at least 97%, at least 99%, identical to SEQ ID NO: 8 which encodes SEQ ID NO: 7. In certain embodiments, the nucleic acid sequence has the nucleic acid sequence of SEQ ID NO: 8 or a sequence at least 70% to 99%, at least 75%, at least 80%, at least 85%, at least 90%, at least 95%, at least 97% at least 99%, identical to about nt 412 to about nt 2211 of SEQ ID NO: 8 which encodes the vp2 capsid protein (about aa 138 to 736) of SEQ ID NO: 7. In certain embodiments, the nucleic acid sequence has the nucleic acid sequence of about nt 607 to about nt 2211 of SEQ ID NO: 8 or a sequence at least 70% to 99%, at least 75%, at least 80%, at least 85%, at least 90%, at least 95%, at least 97%, at least 99%, identical to nt SEQ ID NO: 8 which encodes the vp3 capsid protein (about aa 203 to 736) of SEQ ID NO: 7.

The invention also encompasses nucleic acid sequences encoding mutant AAV3B.AR2.04, in which one or more residues has been altered in order to decrease deamidation, or other modifications which are identified herein. Such nucleic acid sequences can be used in production of mutant AAV3B.AR2.04 capsids.

In certain embodiments, a novel isolated AAV3B.AR2.05 capsid is provided. The nucleic acid sequence encoding the AAV is provided in SEQ ID NO: 10 and the predicted encoded amino acid sequence is provided in SEQ ID NO: 9. Provided herein is an rAAV comprising at least one of the vp1 (aa 1 to 736), vp2 (aa 138 to 736) and the vp3 (aa 203 to 736) of AAV3B.AR2.05 (SEQ ID NO: 9). Also provided herein are rAAV comprising an AAV capsid encoded by at least one of the vp1 (nt 1 to nt 2211), vp2 (nt 412 to nt 2211) and the vp3 (nt 607 to nt 2211) AAV3B.AR2.05 (SEQ ID NO: 10).

In a further aspect, a recombinant adeno-associated virus (rAAV) is provided which comprises: (A) an AAV3B.AR2.05 capsid comprising one or more of: (1) AAV3B.AR2.05 capsid proteins comprising: a heterogeneous population of AAV3B.AR2.05 vp1 proteins selected from: vp1 proteins produced by expression from a nucleic acid sequence which encodes the predicted amino acid sequence of 1 to 736 of SEQ ID NO: 9, vp1 proteins produced from SEQ ID NO: 10, or vp1 proteins produced from a nucleic acid sequence at least 70% identical to SEQ ID NO: 10 which encodes the predicted amino acid sequence of 1 to 736 of SEQ ID NO: 9, a heterogeneous population of AAV3B.AR2.05 vp2 proteins selected from: vp2 proteins produced by expression from a nucleic acid sequence which encodes the predicted amino acid sequence of at least about amino acids 138 to 736 of SEQ ID NO: 9, vp2 proteins produced from a sequence comprising at least nucleotides 412 to 2211 of SEQ ID NO: 10, or vp2 proteins produced from a nucleic acid sequence at least 70% identical to at least nucleotides 412 to 2211 of SEQ ID NO: 10 which encodes the predicted amino acid sequence of at least about amino acids 138 to 736 of SEQ ID NO: 9, a heterogeneous population of AAV3B.AR2.05 vp3 proteins selected from: vp3 proteins produced by expression from a nucleic acid sequence which encodes the predicted amino acid sequence of at least about amino acids 203 to 736 of SEQ ID NO: 9, vp3 proteins produced from a sequence comprising at least nucleotides 607 to 2211 of SEQ ID NO: 10, or vp3 proteins produced from a nucleic acid sequence at least 70% identical to at least nucleotides 607 to 2211 of SEQ ID NO: 10 which encodes the predicted amino acid sequence of at least about amino acids 203 to 736 of SEQ ID NO: 9; and/or (2) a heterogeneous population of vp1 proteins which are the product of a nucleic acid sequence encoding the amino acid sequence of SEQ ID NO: 9, a heterogeneous population of vp2 proteins which are the product of a nucleic acid sequence encoding the amino acid sequence of at least about amino acids 138 to 736 of SEQ ID NO: 9, and a heterogeneous population of vp3 proteins which are the product of a nucleic acid sequence encoding at least amino acids 203 to 736 of SEQ ID NO: 9, wherein: the vp1, vp2 and vp3 proteins contain subpopulations with amino acid modifications comprising at least two highly deamidated asparagines (N) in asparagine-glycine pairs in SEQ ID NO: 9 and optionally further comprising subpopulations comprising other deamidated amino acids, wherein the deamidation results in an amino acid change; and (B) a vector genome in the AAV3B.AR2.05 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.05 capsid comprises: a heterogeneous population of vp1 proteins which are the product of a nucleic acid sequence encoding the amino acid sequence of SEQ ID NO: 9, a heterogeneous population of vp2 proteins which are the product of a nucleic acid sequence encoding the amino acid sequence of at least about amino acids 138 to 736 of SEQ ID NO: 9, and a heterogeneous population of vp3 proteins which are the product of a nucleic acid sequence encoding at least amino acids 203 to 736 of SEQ ID NO: 9.

In certain embodiments, the nucleic acid sequence encoding the AAV3B.AR2.05 vp1 capsid protein is provided in SEQ ID NO: 10. In other embodiments, a nucleic acid sequence of 70% to 99.9% identity to SEQ ID NO: 10 may be selected to express the AAV3B.AR2.05 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 10. However, other nucleic acid sequences which encode the amino acid sequence of SEQ ID NO: 9 may be selected for use in producing rAAV capsids. In certain embodiments, the nucleic acid sequence has the nucleic acid sequence of SEQ ID NO: 10 or a sequence at least 70% to 99% identical, at least 75%, at least 80%, at least 85%, at least 90%, at least 95%, at least 97%, at least 99%, identical to SEQ ID NO: 10 which encodes SEQ ID NO: 9. In certain embodiments, the nucleic acid sequence has the nucleic acid sequence of SEQ ID NO: 10 or a sequence at least 70% to 99%, at least 75%, at least 80%, at least 85%, at least 90%, at least 95%, at least 97%, at least 99%, identical to about nt 412 to about nt 2211 of SEQ ID NO: 10 which encodes the vp2 capsid protein (about aa 138 to 736) of SEQ ID NO: 9. In certain embodiments, the nucleic acid sequence has the nucleic acid sequence of about nt 607 to about nt 2211 of SEQ ID NO: 10 or a sequence at least 70% to 99%, at least 75%, at least 80%, at least 85%, at least 90%, at least 95%, at least 97%, at least 99%, identical to nt SEQ ID NO: 10 which encodes the vp3 capsid protein (about aa 203 to 736) of SEQ ID NO: 9.

The invention also encompasses nucleic acid sequences encoding mutant AAV3B.AR2.05, in which one or more residues has been altered in order to decrease deamidation, or other modifications which are identified herein. Such nucleic acid sequences can be used in production of mutant AAV3B.AR2.05 capsids.

In certain embodiments, a novel isolated AAV3B.AR2.06 capsid is provided. The nucleic acid sequence encoding the AAV is provided in SEQ ID NO: 12 and the predicted encoded amino acid sequence is provided in SEQ ID NO: 11. Provided herein is an rAAV comprising at least one of the vp1 (aa 1 to 736), vp2 (aa 138 to 736) and the vp3 (aa 203 to 736) of AAV3B.AR2.06 (SEQ ID NO: 11). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.06 (SEQ ID NO: 12).

In a further aspect, a recombinant adeno-associated virus (rAAV) is provided which comprises: (A) an AAV3B.AR2.06 capsid comprising one or more of: (1) AAV3B.AR2.06 capsid proteins comprising: a heterogeneous population of AAV3B.AR2.06 vp1 proteins selected from: vp1 proteins produced by expression from a nucleic acid sequence which encodes the predicted amino acid sequence of 1 to 736 of SEQ ID NO: 11, vp1 proteins produced from SEQ ID NO: 12, or vp1 proteins produced from a nucleic acid sequence at least 70% identical to SEQ ID NO: 12 which encodes the predicted amino acid sequence of 1 to 736 of SEQ ID NO: 11, a heterogeneous population of AAV3B.AR2.06 vp2 proteins selected from: vp2 proteins produced by expression from a nucleic acid sequence which encodes the predicted amino acid sequence of at least about amino acids 138 to 736 of SEQ ID NO: 11, vp2 proteins produced from a sequence comprising at least nucleotides 412 to 2211 of SEQ ID NO: 12, or vp2 proteins produced from a nucleic acid sequence at least 70% identical to at least nucleotides 412 to 2211 of SEQ ID NO: 12 which encodes the predicted amino acid sequence of at least about amino acids 138 to 736 of SEQ ID NO: 11, a heterogeneous population of AAV3B.AR2.06 vp3 proteins selected from: vp3 proteins produced by expression from a nucleic acid sequence which encodes the predicted amino acid sequence of at least about amino acids 203 to 736 of SEQ ID NO: 11, vp3 proteins produced from a sequence comprising at least nucleotides 607 to 2211 of SEQ ID NO: 12, or vp3 proteins produced from a nucleic acid sequence at least 70% identical to at least nucleotides 607 to 2211 of SEQ ID NO: 12 which encodes the predicted amino acid sequence of at least about amino acids 203 to 736 of SEQ ID NO: 11; and/or (2) a heterogeneous population of vp1 proteins which are the product of a nucleic acid sequence encoding the amino acid sequence of SEQ ID NO: 11, a heterogeneous population of vp2 proteins which are the product of a nucleic acid sequence encoding the amino acid sequence of at least about amino acids 138 to 736 of SEQ ID NO: 11, and a heterogeneous population of vp3 proteins which are the product of a nucleic acid sequence encoding at least amino acids 203 to 736 of SEQ ID NO: 11, wherein: the vp1, vp2 and vp3 proteins contain subpopulations with amino acid modifications comprising at least two highly deamidated asparagines (N) in asparagine-glycine pairs in SEQ ID NO: 11 and optionally further comprising subpopulations comprising other deamidated amino acids, wherein the deamidation results in an amino acid change; and (B) a vector genome in the AAV3B.AR2.06 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.06 capsid comprises: a heterogeneous population of vp1 proteins which are the product of a nucleic acid sequence encoding the amino acid sequence of SEQ ID NO: 11, a heterogeneous population of vp2 proteins which are the product of a nucleic acid sequence encoding the amino acid sequence of at least about amino acids 138 to 736 of SEQ ID NO: 11, and a heterogeneous population of vp3 proteins which are the product of a nucleic acid sequence encoding at least amino acids 203 to 736 of SEQ ID NO: 11.

In certain embodiments, the nucleic acid sequence encoding the AAV3B.AR2.06 vp1 capsid protein is provided in SEQ ID NO: 12. In other embodiments, a nucleic acid sequence of 70% to 99.9% identity to SEQ ID NO: 12 may be selected to express the AAV3B.AR2.06 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 12. However, other nucleic acid sequences which encode the amino acid sequence of SEQ ID NO: 11 may be selected for use in producing rAAV capsids. In certain embodiments, the nucleic acid sequence has the nucleic acid sequence of SEQ ID NO: 12 or a sequence at least 70% to 99% identical, at least 75%, at least 80%, at least 85%, at least 90%, at least 95%, at least 97%, at least 99%, identical to SEQ ID NO: 12 which encodes SEQ ID NO: 11. In certain embodiments, the nucleic acid sequence has the nucleic acid sequence of SEQ ID NO: 12 or a sequence at least 70% to 99%, at least 75%, at least 80%, at least 85%, at least 90%, at least 95%, at least 97%, at least 99%, identical to about nt 412 to about nt 2211 of SEQ ID NO: 12 which encodes the vp2 capsid protein (about aa 138 to 736) of SEQ ID NO: 11. In certain embodiments, the nucleic acid sequence has the nucleic acid sequence of about nt 607 to about nt 2211 of SEQ ID NO: 12 or a sequence at least 70% to 99%, at least 75%, at least 80%, at least 85%, at least 90%, at least 95%, at least 97%, at least 99%, identical to nt SEQ ID NO: 12 which encodes the vp3 capsid protein (about aa 203 to 736) of SEQ ID NO: 11.

The invention also encompasses nucleic acid sequences encoding mutant AAV3B.AR2.06, in which one or more residues has been altered in order to decrease deamidation, or other modifications which are identified herein. Such nucleic acid sequences can be used in production of mutant AAV3B.AR2.06 capsids.

In certain embodiments, a novel isolated AAV3B.AR2.07 capsid is provided. The nucleic acid sequence encoding the AAV is provided in SEQ ID NO: 14 and the predicted encoded amino acid sequence is provided in SEQ ID NO: 13. Provided herein is an rAAV comprising at least one of the vp1 (aa 1 to 736), vp2 (aa 138 to 736) and the vp3 (aa 203 to 736) of AAV3B.AR2.07 (SEQ ID NO: 13). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.07 (SEQ ID NO: 14).

In a further aspect, a recombinant adeno-associated virus (rAAV) is provided which comprises: (A) an AAV3B.AR2.07 capsid comprising one or more of: (1) AAV3B.AR2.07 capsid proteins comprising: a heterogeneous population of AAV3B.AR2.07 vp1 proteins selected from: vp1 proteins produced by expression from a nucleic acid sequence which encodes the predicted amino acid sequence of 1 to 736 of SEQ ID NO: 13, vp1 proteins produced from SEQ ID NO: 14, or vp1 proteins produced from a nucleic acid sequence at least 70% identical to SEQ ID NO: 14 which encodes the predicted amino acid sequence of 1 to 736 of SEQ ID NO: 13, a heterogeneous population of AAV3B.AR2.07 vp2 proteins selected from: vp2 proteins produced by expression from a nucleic acid sequence which encodes the predicted amino acid sequence of at least about amino acids 138 to 736 of SEQ ID NO: 13, vp2 proteins produced from a sequence comprising at least nucleotides 412 to 2211 of SEQ ID NO: 14, or vp2 proteins produced from a nucleic acid sequence at least 70% identical to at least nucleotides 412 to 2211 of SEQ ID NO: 14 which encodes the predicted amino acid sequence of at least about amino acids 138 to 736 of SEQ ID NO: 13, a heterogeneous population of AAV3B.AR2.07 vp3 proteins selected from: vp3 proteins produced by expression from a nucleic acid sequence which encodes the predicted amino acid sequence of at least about amino acids 203 to 736 of SEQ ID NO: 13, vp3 proteins produced from a sequence comprising at least nucleotides 607 to 2211 of SEQ ID NO: 14, or vp3 proteins produced from a nucleic acid sequence at least 70% identical to at least nucleotides 607 to 2211 of SEQ ID NO: 14 which encodes the predicted amino acid sequence of at least about amino acids 203 to 736 of SEQ ID NO: 13; and/or (2) a heterogeneous population of vp1 proteins which are the product of a nucleic acid sequence encoding the amino acid sequence of SEQ ID NO: 13, a heterogeneous population of vp2 proteins which are the product of a nucleic acid sequence encoding the amino acid sequence of at least about amino acids 138 to 736 of SEQ ID NO: 13, and a heterogeneous population of vp3 proteins which are the product of a nucleic acid sequence encoding at least amino acids 203 to 736 of SEQ ID NO: 13, wherein: the vp1, vp2 and vp3 proteins contain subpopulations with amino acid modifications comprising at least two highly deamidated asparagines (N) in asparagine-glycine pairs in SEQ ID NO: 13 and optionally further comprising subpopulations comprising other deamidated amino acids, wherein the deamidation results in an amino acid change; and (B) a vector genome in the AAV3B.AR2.07 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.07 capsid comprises: a heterogeneous population of vp1 proteins which are the product of a nucleic acid sequence encoding the amino acid sequence of SEQ ID NO: 13, a heterogeneous population of vp2 proteins which are the product of a nucleic acid sequence encoding the amino acid sequence of at least about amino acids 138 to 736 of SEQ ID NO: 13, and a heterogeneous population of vp3 proteins which are the product of a nucleic acid sequence encoding at least amino acids 203 to 736 of SEQ ID NO: 13.

In certain embodiments, the nucleic acid sequence encoding the AAV3B.AR2.07 vp1 capsid protein is provided in SEQ ID NO: 14. In other embodiments, a nucleic acid sequence of 70% to 99.9% identity to SEQ ID NO: 14 may be selected to express the AAV3B.AR2.07 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 14. However, other nucleic acid sequences which encode the amino acid sequence of SEQ ID NO: 13 may be selected for use in producing rAAV capsids. In certain embodiments, the nucleic acid sequence has the nucleic acid sequence of SEQ ID NO: 14 or a sequence at least 70% to 99% identical, at least 75%, at least 80%, at least 85%, at least 90%, at least 95%, at least 97%, at least 99%, identical to SEQ ID NO: 14 which encodes SEQ ID NO: 13. In certain embodiments, the nucleic acid sequence has the nucleic acid sequence of SEQ ID NO: 14 or a sequence at least 70% to 99%, at least 75%, at least 80%, at least 85%, at least 90%, at least 95%, at least 97%, at least 99%, identical to about nt 412 to about nt 2211 of SEQ ID NO: 14 which encodes the vp2 capsid protein (about aa 138 to 736) of SEQ ID NO: 13. In certain embodiments, the nucleic acid sequence has the nucleic acid sequence of about nt 607 to about nt 2211 of SEQ ID NO: 14 or a sequence at least 70% to 99%, at least 75%, at least 80%, at least 85%, at least 90%, at least 95%, at least 97%, at least 99%, identical to nt SEQ ID NO: 14 which encodes the vp3 capsid protein (about aa 203 to 736) of SEQ ID NO: 13.

The invention also encompasses nucleic acid sequences encoding mutant AAV3B.AR2.07, in which one or more residues has been altered in order to decrease deamidation, or other modifications which are identified herein. Such nucleic acid sequences can be used in production of mutant AAV3B.AR2.07 capsids.

In certain embodiments, a novel isolated AAV3B.AR2.08 capsid is provided. The nucleic acid sequence encoding the AAV is provided in SEQ ID NO: 16 and the predicted encoded amino acid sequence is provided in SEQ ID NO: 15. Provided herein is an rAAV comprising at least one of the vp1 (aa 1 to 736), vp2 (aa 138 to 736) and the vp3 (aa 203 to 736) of AAV3B.AR2.08 (SEQ ID NO: 15). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.08 (SEQ ID NO: 16).

In a further aspect, a recombinant adeno-associated virus (rAAV) is provided which comprises: (A) an AAV3B.AR2.08 capsid comprising one or more of: (1) AAV3B.AR2.08 capsid proteins comprising: a heterogeneous population of AAV3B.AR2.08 vp1 proteins selected from: vp1 proteins produced by expression from a nucleic acid sequence which encodes the predicted amino acid sequence of 1 to 736 of SEQ ID NO: 15, vp1 proteins produced from SEQ ID NO: 16, or vp1 proteins produced from a nucleic acid sequence at least 70% identical to SEQ ID NO: 16 which encodes the predicted amino acid sequence of 1 to 736 of SEQ ID NO: 15, a heterogeneous population of AAV3B.AR2.08 vp2 proteins selected from: vp2 proteins produced by expression from a nucleic acid sequence which encodes the predicted amino acid sequence of at least about amino acids 138 to 736 of SEQ ID NO: 15, vp2 proteins produced from a sequence comprising at least nucleotides 412 to 2211 of SEQ ID NO: 16, or vp2 proteins produced from a nucleic acid sequence at least 70% identical to at least nucleotides 412 to 2211 of SEQ ID NO: 16 which encodes the predicted amino acid sequence of at least about amino acids 138 to 736 of SEQ ID NO: 15, a heterogeneous population of AAV3B.AR2.08 vp3 proteins selected from: vp3 proteins produced by expression from a nucleic acid sequence which encodes the predicted amino acid sequence of at least about amino acids 203 to 736 of SEQ ID NO: 15, vp3 proteins produced from a sequence comprising at least nucleotides 607 to 2211 of SEQ ID NO: 16, or vp3 proteins produced from a nucleic acid sequence at least 70% identical to at least nucleotides 607 to 2211 of SEQ ID NO: 16 which encodes the predicted amino acid sequence of at least about amino acids 203 to 736 of SEQ ID NO: 15; and/or (2) a heterogeneous population of vp1 proteins which are the product of a nucleic acid sequence encoding the amino acid sequence of SEQ ID NO: 15, a heterogeneous population of vp2 proteins which are the product of a nucleic acid sequence encoding the amino acid sequence of at least about amino acids 138 to 736 of SEQ ID NO: 15, and a heterogeneous population of vp3 proteins which are the product of a nucleic acid sequence encoding at least amino acids 203 to 736 of SEQ ID NO: 15, wherein: the vp1, vp2 and vp3 proteins contain subpopulations with amino acid modifications comprising at least two highly deamidated asparagines (N) in asparagine-glycine pairs in SEQ ID NO: 15 and optionally further comprising subpopulations comprising other deamidated amino acids, wherein the deamidation results in an amino acid change; and (B) a vector genome in the AAV3B.AR2.08 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.08 capsid comprises: a heterogeneous population of vp1 proteins which are the product of a nucleic acid sequence encoding the amino acid sequence of SEQ ID NO: 15, a heterogeneous population of vp2 proteins which are the product of a nucleic acid sequence encoding the amino acid sequence of at least about amino acids 138 to 736 of SEQ ID NO: 15, and a heterogeneous population of vp3 proteins which are the product of a nucleic acid sequence encoding at least amino acids 203 to 736 of SEQ ID NO: 15.

In certain embodiments, the nucleic acid sequence encoding the AAV3B.AR2.08 vp1 capsid protein is provided in SEQ ID NO: 16. In other embodiments, a nucleic acid sequence of 70% to 99.9% identity to SEQ ID NO: 16 may be selected to express the AAV3B.AR2.08 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 16. However, other nucleic acid sequences which encode the amino acid sequence of SEQ ID NO: 15 may be selected for use in producing rAAV capsids. In certain embodiments, the nucleic acid sequence has the nucleic acid sequence of SEQ ID NO: 16 or a sequence at least 70% to 99% identical, at least 75%, at least 80%, at least 85%, at least 90%, at least 95%, at least 97%, at least 99%, identical to SEQ ID NO: 16 which encodes SEQ ID NO: 15. In certain embodiments, the nucleic acid sequence has the nucleic acid sequence of SEQ ID NO: 16 or a sequence at least 70% to 99%, at least 75%, at least 80%, at least 85%, at least 90%, at least 95%, at least 97%, at least 99%, identical to about nt 412 to about nt 2211 of SEQ ID NO: 16 which encodes the vp2 capsid protein (about aa 138 to 736) of SEQ ID NO: 15. In certain embodiments, the nucleic acid sequence has the nucleic acid sequence of about nt 607 to about nt 2211 of SEQ ID NO: 16 or a sequence at least 70% to 99%, at least 75%, at least 80%, at least 85%, at least 90%, at least 95%, at least 97%, at least 99%, identical to nt SEQ ID NO: 16 which encodes the vp3 capsid protein (about aa 203 to 736) of SEQ ID NO: 15.

The invention also encompasses nucleic acid sequences encoding mutant AAV3B.AR2.08, in which one or more residues has been altered in order to decrease deamidation, or other modifications which are identified herein. Such nucleic acid sequences can be used in production of mutant AAV3B.AR2.08 capsids.

In certain embodiments, a novel isolated AAV3B.AR2.10 capsid is provided. The nucleic acid sequence encoding the AAV is provided in SEQ ID NO: 18 and the predicted encoded amino acid sequence is provided in SEQ ID NO: 17. Provided herein is an rAAV comprising at least one of the vp1 (aa 1 to 736), vp2 (aa 138 to 736) and the vp3 (aa 203 to 736) of AAV3B.AR2.10 (SEQ ID NO: 17). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.10 (SEQ ID NO: 18).

In a further aspect, a recombinant adeno-associated virus (rAAV) is provided which comprises: (A) an AAV3B.AR2.10 capsid comprising one or more of: (1) AAV3B.AR2.10 capsid proteins comprising: a heterogeneous population of AAV3B.AR2.10 vp1 proteins selected from: vp1 proteins produced by expression from a nucleic acid sequence which encodes the predicted amino acid sequence of 1 to 736 of SEQ ID NO: 17, vp1 proteins produced from SEQ ID NO: 18, or vp1 proteins produced from a nucleic acid sequence at least 70% identical to SEQ ID NO: 18 which encodes the predicted amino acid sequence of 1 to 736 of SEQ ID NO: 17, a heterogeneous population of AAV3B.AR2.10 vp2 proteins selected from: vp2 proteins produced by expression from a nucleic acid sequence which encodes the predicted amino acid sequence of at least about amino acids 138 to 736 of SEQ ID NO: 17, vp2 proteins produced from a sequence comprising at least nucleotides 412 to 2211 of SEQ ID NO: 18, or vp2 proteins produced from a nucleic acid sequence at least 70% identical to at least nucleotides 412 to 2211 of SEQ ID NO: 18 which encodes the predicted amino acid sequence of at least about amino acids 138 to 736 of SEQ ID NO: 17, a heterogeneous population of AAV3B.AR2.10 vp3 proteins selected from: vp3 proteins produced by expression from a nucleic acid sequence which encodes the predicted amino acid sequence of at least about amino acids 203 to 736 of SEQ ID NO: 17, vp3 proteins produced from a sequence comprising at least nucleotides 607 to 2211 of SEQ ID NO: 18, or vp3 proteins produced from a nucleic acid sequence at least 70% identical to at least nucleotides 607 to 2211 of SEQ ID NO: 18 which encodes the predicted amino acid sequence of at least about amino acids 203 to 736 of SEQ ID NO: 17; and/or (2) a heterogeneous population of vp1 proteins which are the product of a nucleic acid sequence encoding the amino acid sequence of SEQ ID NO: 17, a heterogeneous population of vp2 proteins which are the product of a nucleic acid sequence encoding the amino acid sequence of at least about amino acids 138 to 736 of SEQ ID NO: 17, and a heterogeneous population of vp3 proteins which are the product of a nucleic acid sequence encoding at least amino acids 203 to 736 of SEQ ID NO: 17, wherein: the vp1, vp2 and vp3 proteins contain subpopulations with amino acid modifications comprising at least two highly deamidated asparagines (N) in asparagine-glycine pairs in SEQ ID NO: 17 and optionally further comprising subpopulations comprising other deamidated amino acids, wherein the deamidation results in an amino acid change; and (B) a vector genome in the AAV3B.AR2.10 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.10 capsid comprises: a heterogeneous population of vp1 proteins which are the product of a nucleic acid sequence encoding the amino acid sequence of SEQ ID NO: 17, a heterogeneous population of vp2 proteins which are the product of a nucleic acid sequence encoding the amino acid sequence of at least about amino acids 138 to 736 of SEQ ID NO: 17, and a heterogeneous population of vp3 proteins which are the product of a nucleic acid sequence encoding at least amino acids 203 to 736 of SEQ ID NO: 17.

In certain embodiments, the nucleic acid sequence encoding the AAV3B.AR2.10 vp1 capsid protein is provided in SEQ ID NO: 18. In other embodiments, a nucleic acid sequence of 70% to 99.9% identity to SEQ ID NO: 18 may be selected to express the AAV3B.AR2.10 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 18. However, other nucleic acid sequences which encode the amino acid sequence of SEQ ID NO: 17 may be selected for use in producing rAAV capsids. In certain embodiments, the nucleic acid sequence has the nucleic acid sequence of SEQ ID NO: 18 or a sequence at least 70% to 99% identical, at least 75%, at least 80%, at least 85%, at least 90%, at least 95%, at least 97%, at least 99%, identical to SEQ ID NO: 18 which encodes SEQ ID NO: 17. In certain embodiments, the nucleic acid sequence has the nucleic acid sequence of SEQ ID NO: 18 or a sequence at least 70% to 99%, at least 75%, at least 80%, at least 85%, at least 90%, at least 95%, at least 97%, at least 99%, identical to about nt 412 to about nt 2211 of SEQ ID NO: 18 which encodes the vp2 capsid protein (about aa 138 to 736) of SEQ ID NO: 17. In certain embodiments, the nucleic acid sequence has the nucleic acid sequence of about nt 607 to about nt 2211 of SEQ ID NO: 18 or a sequence at least 70% to 99%, at least 75%, at least 80%, at least 85%, at least 90%, at least 95%, at least 97%, at least 99%, identical to nt SEQ ID NO: 18 which encodes the vp3 capsid protein (about aa 203 to 736) of SEQ ID NO: 17.

The invention also encompasses nucleic acid sequences encoding mutant AAV3B.AR2.10, in which one or more residues has been altered in order to decrease deamidation, or other modifications which are identified herein. Such nucleic acid sequences can be used in production of mutant AAV3B.AR2.10 capsids.

In certain embodiments, a novel isolated AAV3B.AR2.11 capsid is provided. The nucleic acid sequence encoding the AAV is provided in SEQ ID NO: 20 and the predicted encoded amino acid sequence is provided in SEQ ID NO: 19. Provided herein is an rAAV comprising at least one of the vp1 (aa 1 to 736), vp2 (aa 138 to 736) and the vp3 (aa 203 to 736) of AAV3B.AR2.11 (SEQ ID NO: 19). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.11 (SEQ ID NO: 20).

In a further aspect, a recombinant adeno-associated virus (rAAV) is provided which comprises: (A) an AAV3B.AR2.11 capsid comprising one or more of: (1) AAV3B.AR2.11 capsid proteins comprising: a heterogeneous population of AAV3B.AR2.11 vp1 proteins selected from: vp1 proteins produced by expression from a nucleic acid sequence which encodes the predicted amino acid sequence of 1 to 736 of SEQ ID NO: 19, vp1 proteins produced from SEQ ID NO: 20, or vp1 proteins produced from a nucleic acid sequence at least 70% identical to SEQ ID NO: 20 which encodes the predicted amino acid sequence of 1 to 736 of SEQ ID NO: 19, a heterogeneous population of AAV3B.AR2.11 vp2 proteins selected from: vp2 proteins produced by expression from a nucleic acid sequence which encodes the predicted amino acid sequence of at least about amino acids 138 to 736 of SEQ ID NO: 19, vp2 proteins produced from a sequence comprising at least nucleotides 412 to 2211 of SEQ ID NO: 20, or vp2 proteins produced from a nucleic acid sequence at least 70% identical to at least nucleotides 412 to 2211 of SEQ ID NO: 20 which encodes the predicted amino acid sequence of at least about amino acids 138 to 736 of SEQ ID NO: 19, a heterogeneous population of AAV3B.AR2.11 vp3 proteins selected from: vp3 proteins produced by expression from a nucleic acid sequence which encodes the predicted amino acid sequence of at least about amino acids 203 to 736 of SEQ ID NO: 19, vp3 proteins produced from a sequence comprising at least nucleotides 607 to 2211 of SEQ ID NO: 20, or vp3 proteins produced from a nucleic acid sequence at least 70% identical to at least nucleotides 607 to 2211 of SEQ ID NO: 20 which encodes the predicted amino acid sequence of at least about amino acids 203 to 736 of SEQ ID NO: 19; and/or (2) a heterogeneous population of vp1 proteins which are the product of a nucleic acid sequence encoding the amino acid sequence of SEQ ID NO: 19, a heterogeneous population of vp2 proteins which are the product of a nucleic acid sequence encoding the amino acid sequence of at least about amino acids 138 to 736 of SEQ ID NO: 19, and a heterogeneous population of vp3 proteins which are the product of a nucleic acid sequence encoding at least amino acids 203 to 736 of SEQ ID NO: 19, wherein: the vp1, vp2 and vp3 proteins contain subpopulations with amino acid modifications comprising at least two highly deamidated asparagines (N) in asparagine-glycine pairs in SEQ ID NO: 19 and optionally further comprising subpopulations comprising other deamidated amino acids, wherein the deamidation results in an amino acid change; and (B) a vector genome in the AAV3B.AR2.11 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.11 capsid comprises: a heterogeneous population of vp1 proteins which are the product of a nucleic acid sequence encoding the amino acid sequence of SEQ ID NO: 19, a heterogeneous population of vp2 proteins which are the product of a nucleic acid sequence encoding the amino acid sequence of at least about amino acids 138 to 736 of SEQ ID NO: 19, and a heterogeneous population of vp3 proteins which are the product of a nucleic acid sequence encoding at least amino acids 203 to 736 of SEQ ID NO: 19.

In certain embodiments, the nucleic acid sequence encoding the AAV3B.AR2.11 vp1 capsid protein is provided in SEQ ID NO: 20. In other embodiments, a nucleic acid sequence of 70% to 99.9% identity to SEQ ID NO: 20 may be selected to express the AAV3B.AR2.11 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 20. However, other nucleic acid sequences which encode the amino acid sequence of SEQ ID NO: 19 may be selected for use in producing rAAV capsids. In certain embodiments, the nucleic acid sequence has the nucleic acid sequence of SEQ ID NO: 20 or a sequence at least 70% to 99% identical, at least 75%, at least 80%, at least 85%, at least 90%, at least 95%, at least 97%, at least 99%, identical to SEQ ID NO: 20 which encodes SEQ ID NO: 19. In certain embodiments, the nucleic acid sequence has the nucleic acid sequence of SEQ ID NO: 20 or a sequence at least 70% to 99%, at least 75%, at least 80%, at least 85%, at least 90%, at least 95%, at least 97%, at least 99%, identical to about nt 412 to about nt 2211 of SEQ ID NO: 20 which encodes the vp2 capsid protein (about aa 138 to 736) of SEQ ID NO: 19. In certain embodiments, the nucleic acid sequence has the nucleic acid sequence of about nt 607 to about nt 2211 of SEQ ID NO: 20 or a sequence at least 70% to 99%, at least 75%, at least 80%, at least 85%, at least 90%, at least 95%, at least 97%, at least 99%, identical to nt SEQ ID NO: 20 which encodes the vp3 capsid protein (about aa 203 to 736) of SEQ ID NO: 19.

The invention also encompasses nucleic acid sequences encoding mutant AAV3B.AR2.11, in which one or more residues has been altered in order to decrease deamidation, or other modifications which are identified herein. Such nucleic acid sequences can be used in production of mutant AAV3B.AR2.11 capsids.

In certain embodiments, a novel isolated AAV3B.AR2.12 capsid is provided. The nucleic acid sequence encoding the AAV is provided in SEQ ID NO: 22 and the predicted encoded amino acid sequence is provided in SEQ ID NO: 21. Provided herein is an rAAV comprising at least one of the vp1 (aa 1 to 736), vp2 (aa 138 to 736) and the vp3 (aa 203 to 736) of AAV3B.AR2.12 (SEQ ID NO: 21). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.12 (SEQ ID NO: 22).

In a further aspect, a recombinant adeno-associated virus (rAAV) is provided which comprises: (A) an AAV3B.AR2.12 capsid comprising one or more of: (1) AAV3B.AR2.12 capsid proteins comprising: a heterogeneous population of AAV3B.AR2.12 vp1 proteins selected from: vp1 proteins produced by expression from a nucleic acid sequence which encodes the predicted amino acid sequence of 1 to 736 of SEQ ID NO: 21, vp1 proteins produced from SEQ ID NO: 22, or vp1 proteins produced from a nucleic acid sequence at least 70% identical to SEQ ID NO: 22 which encodes the predicted amino acid sequence of 1 to 736 of SEQ ID NO: 21, a heterogeneous population of AAV3B.AR2.12 vp2 proteins selected from: vp2 proteins produced by expression from a nucleic acid sequence which encodes the predicted amino acid sequence of at least about amino acids 138 to 736 of SEQ ID NO: 21, vp2 proteins produced from a sequence comprising at least nucleotides 412 to 2211 of SEQ ID NO: 22, or vp2 proteins produced from a nucleic acid sequence at least 70% identical to at least nucleotides 412 to 2211 of SEQ ID NO: 22 which encodes the predicted amino acid sequence of at least about amino acids 138 to 736 of SEQ ID NO: 21, a heterogeneous population of AAV3B.AR2.12 vp3 proteins selected from: vp3 proteins produced by expression from a nucleic acid sequence which encodes the predicted amino acid sequence of at least about amino acids 203 to 736 of SEQ ID NO: 21, vp3 proteins produced from a sequence comprising at least nucleotides 607 to 2211 of SEQ ID NO: 22, or vp3 proteins produced from a nucleic acid sequence at least 70% identical to at least nucleotides 607 to 2211 of SEQ ID NO: 22 which encodes the predicted amino acid sequence of at least about amino acids 203 to 736 of SEQ ID NO: 21; and/or (2) a heterogeneous population of vp1 proteins which are the product of a nucleic acid sequence encoding the amino acid sequence of SEQ ID NO: 21, a heterogeneous population of vp2 proteins which are the product of a nucleic acid sequence encoding the amino acid sequence of at least about amino acids 138 to 736 of SEQ ID NO: 21, and a heterogeneous population of vp3 proteins which are the product of a nucleic acid sequence encoding at least amino acids 203 to 736 of SEQ ID NO: 21, wherein: the vp1, vp2 and vp3 proteins contain subpopulations with amino acid modifications comprising at least two highly deamidated asparagines (N) in asparagine-glycine pairs in SEQ ID NO: 21 and optionally further comprising subpopulations comprising other deamidated amino acids, wherein the deamidation results in an amino acid change; and (B) a vector genome in the AAV3B.AR2.12 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.12 capsid comprises: a heterogeneous population of vp1 proteins which are the product of a nucleic acid sequence encoding the amino acid sequence of SEQ ID NO: 21, a heterogeneous population of vp2 proteins which are the product of a nucleic acid sequence encoding the amino acid sequence of at least about amino acids 138 to 736 of SEQ ID NO: 21, and a heterogeneous population of vp3 proteins which are the product of a nucleic acid sequence encoding at least amino acids 203 to 736 of SEQ ID NO: 21.

In certain embodiments, the nucleic acid sequence encoding the AAV3B.AR2.12 vp1 capsid protein is provided in SEQ ID NO: 22. In other embodiments, a nucleic acid sequence of 70% to 99.9% identity to SEQ ID NO: 22 may be selected to express the AAV3B.AR2.12 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 22. However, other nucleic acid sequences which encode the amino acid sequence of SEQ ID NO: 21 may be selected for use in producing rAAV capsids. In certain embodiments, the nucleic acid sequence has the nucleic acid sequence of SEQ ID NO: 22 or a sequence at least 70% to 99% identical, at least 75%, at least 80%, at least 85%, at least 90%, at least 95%, at least 97%, at least 99%, identical to SEQ ID NO: 22 which encodes SEQ ID NO: 21. In certain embodiments, the nucleic acid sequence has the nucleic acid sequence of SEQ ID NO: 22 or a sequence at least 70% to 99%, at least 75%, at least 80%, at least 85%, at least 90%, at least 95%, at least 97%, at least 99%, identical to about nt 412 to about nt 2211 of SEQ ID NO: 22 which encodes the vp2 capsid protein (about aa 138 to 736) of SEQ ID NO: 21. In certain embodiments, the nucleic acid sequence has the nucleic acid sequence of about nt 607 to about nt 2211 of SEQ ID NO: 22 or a sequence at least 70% to 99%, at least 75%, at least 80%, at least 85%, at least 90%, at least 95%, at least 97%, at least 99%, identical to nt SEQ ID NO: 22 which encodes the vp3 capsid protein (about aa 203 to 736) of SEQ ID NO: 21.

The invention also encompasses nucleic acid sequences encoding mutant AAV3B.AR2.12, in which one or more residues has been altered in order to decrease deamidation, or other modifications which are identified herein. Such nucleic acid sequences can be used in production of mutant AAV3B.AR2.12 capsids.

In certain embodiments, a novel isolated AAV3B.AR2.13 capsid is provided. The nucleic acid sequence encoding the AAV is provided in SEQ ID NO: 24 and the predicted encoded amino acid sequence is provided in SEQ ID NO: 23. Provided herein is an rAAV comprising at least one of the vp1 (aa 1 to 736), vp2 (aa 138 to 736) and the vp3 (aa 203 to 736) of AAV3B.AR2.13 (SEQ ID NO: 23). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.13 (SEQ ID NO: 24).

In a further aspect, a recombinant adeno-associated virus (rAAV) is provided which comprises: (A) an AAV3B.AR2.13 capsid comprising one or more of: (1) AAV3B.AR2.13 capsid proteins comprising: a heterogeneous population of AAV3B.AR2.13 vp1 proteins selected from: vp1 proteins produced by expression from a nucleic acid sequence which encodes the predicted amino acid sequence of 1 to 736 of SEQ ID NO: 23, vp1 proteins produced from SEQ ID NO: 24, or vp1 proteins produced from a nucleic acid sequence at least 70% identical to SEQ ID NO: 24 which encodes the predicted amino acid sequence of 1 to 736 of SEQ ID NO: 23, a heterogeneous population of AAV3B.AR2.13 vp2 proteins selected from: vp2 proteins produced by expression from a nucleic acid sequence which encodes the predicted amino acid sequence of at least about amino acids 138 to 736 of SEQ ID NO: 23, vp2 proteins produced from a sequence comprising at least nucleotides 412 to 2211 of SEQ ID NO: 24, or vp2 proteins produced from a nucleic acid sequence at least 70% identical to at least nucleotides 412 to 2211 of SEQ ID NO: 24 which encodes the predicted amino acid sequence of at least about amino acids 138 to 736 of SEQ ID NO: 23, a heterogeneous population of AAV3B.AR2.13 vp3 proteins selected from: vp3 proteins produced by expression from a nucleic acid sequence which encodes the predicted amino acid sequence of at least about amino acids 203 to 736 of SEQ ID NO: 23, vp3 proteins produced from a sequence comprising at least nucleotides 607 to 2211 of SEQ ID NO: 24, or vp3 proteins produced from a nucleic acid sequence at least 70% identical to at least nucleotides 607 to 2211 of SEQ ID NO: 24 which encodes the predicted amino acid sequence of at least about amino acids 203 to 736 of SEQ ID NO: 23; and/or (2) a heterogeneous population of vp1 proteins which are the product of a nucleic acid sequence encoding the amino acid sequence of SEQ ID NO: 23, a heterogeneous population of vp2 proteins which are the product of a nucleic acid sequence encoding the amino acid sequence of at least about amino acids 138 to 736 of SEQ ID NO: 23, and a heterogeneous population of vp3 proteins which are the product of a nucleic acid sequence encoding at least amino acids 203 to 736 of SEQ ID NO: 23, wherein: the vp1, vp2 and vp3 proteins contain subpopulations with amino acid modifications comprising at least two highly deamidated asparagines (N) in asparagine-glycine pairs in SEQ ID NO: 23 and optionally further comprising subpopulations comprising other deamidated amino acids, wherein the deamidation results in an amino acid change; and (B) a vector genome in the AAV3B.AR2.13 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.13 capsid comprises: a heterogeneous population of vp1 proteins which are the product of a nucleic acid sequence encoding the amino acid sequence of SEQ ID NO: 23, a heterogeneous population of vp2 proteins which are the product of a nucleic acid sequence encoding the amino acid sequence of at least about amino acids 138 to 736 of SEQ ID NO: 23, and a heterogeneous population of vp3 proteins which are the product of a nucleic acid sequence encoding at least amino acids 203 to 736 of SEQ ID NO: 23.

In certain embodiments, the nucleic acid sequence encoding the AAV3B.AR2.13 vp1 capsid protein is provided in SEQ ID NO: 24. In other embodiments, a nucleic acid sequence of 70% to 99.9% identity to SEQ ID NO: 24 may be selected to express the AAV3B.AR2.13 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 24. However, other nucleic acid sequences which encode the amino acid sequence of SEQ ID NO: 23 may be selected for use in producing rAAV capsids. In certain embodiments, the nucleic acid sequence has the nucleic acid sequence of SEQ ID NO: 24 or a sequence at least 70% to 99% identical, at least 75%, at least 80%, at least 85%, at least 90%, at least 95%, at least 97%, at least 99%, identical to SEQ ID NO: 24 which encodes SEQ ID NO: 23. In certain embodiments, the nucleic acid sequence has the nucleic acid sequence of SEQ ID NO: 24 or a sequence at least 70% to 99%, at least 75%, at least 80%, at least 85%, at least 90%, at least 95%, at least 97%, at least 99%, identical to about nt 412 to about nt 2211 of SEQ ID NO: 24 which encodes the vp2 capsid protein (about aa 138 to 736) of SEQ ID NO: 23. In certain embodiments, the nucleic acid sequence has the nucleic acid sequence of about nt 607 to about nt 2211 of SEQ ID NO: 24 or a sequence at least 70% to 99%, at least 75%, at least 80%, at least 85%, at least 90%, at least 95%, at least 97%, at least 99%, identical to nt SEQ ID NO: 24 which encodes the vp3 capsid protein (about aa 203 to 736) of SEQ ID NO: 23.

The invention also encompasses nucleic acid sequences encoding mutant AAV3B.AR2.13, in which one or more residues has been altered in order to decrease deamidation, or other modifications which are identified herein. Such nucleic acid sequences can be used in production of mutant AAV3B.AR2.13 capsids.

In certain embodiments, a novel isolated AAV3B.AR2.14 capsid is provided. The nucleic acid sequence encoding the AAV is provided in SEQ ID NO: 26 and the predicted encoded amino acid sequence is provided in SEQ ID NO: 25. Provided herein is an rAAV comprising at least one of the vp1 (aa 1 to 736), vp2 (aa 138 to 736) and the vp3 (aa 203 to 736) of AAV3B.AR2.14 (SEQ ID NO: 25). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.14 (SEQ ID NO: 26).

In a further aspect, a recombinant adeno-associated virus (rAAV) is provided which comprises: (A) an AAV3B.AR2.14 capsid comprising one or more of: (1) AAV3B.AR2.14 capsid proteins comprising: a heterogeneous population of AAV3B.AR2.14 vp1 proteins selected from: vp1 proteins produced by expression from a nucleic acid sequence which encodes the predicted amino acid sequence of 1 to 736 of SEQ ID NO: 25, vp1 proteins produced from SEQ ID NO: 26, or vp1 proteins produced from a nucleic acid sequence at least 70% identical to SEQ ID NO: 26 which encodes the predicted amino acid sequence of 1 to 736 of SEQ ID NO: 25, a heterogeneous population of AAV3B.AR2.14 vp2 proteins selected from: vp2 proteins produced by expression from a nucleic acid sequence which encodes the predicted amino acid sequence of at least about amino acids 138 to 736 of SEQ ID NO: 25, vp2 proteins produced from a sequence comprising at least nucleotides 412 to 2211 of SEQ ID NO: 26, or vp2 proteins produced from a nucleic acid sequence at least 70% identical to at least nucleotides 412 to 2211 of SEQ ID NO: 26 which encodes the predicted amino acid sequence of at least about amino acids 138 to 736 of SEQ ID NO: 25, a heterogeneous population of AAV3B.AR2.14 vp3 proteins selected from: vp3 proteins produced by expression from a nucleic acid sequence which encodes the predicted amino acid sequence of at least about amino acids 203 to 736 of SEQ ID NO: 25, vp3 proteins produced from a sequence comprising at least nucleotides 607 to 2211 of SEQ ID NO: 26, or vp3 proteins produced from a nucleic acid sequence at least 70% identical to at least nucleotides 607 to 2211 of SEQ ID NO: 26 which encodes the predicted amino acid sequence of at least about amino acids 203 to 736 of SEQ ID NO: 25; and/or (2) a heterogeneous population of vp1 proteins which are the product of a nucleic acid sequence encoding the amino acid sequence of SEQ ID NO: 25, a heterogeneous population of vp2 proteins which are the product of a nucleic acid sequence encoding the amino acid sequence of at least about amino acids 138 to 736 of SEQ ID NO: 25, and a heterogeneous population of vp3 proteins which are the product of a nucleic acid sequence encoding at least amino acids 203 to 736 of SEQ ID NO: 25, wherein: the vp1, vp2 and vp3 proteins contain subpopulations with amino acid modifications comprising at least two highly deamidated asparagines (N) in asparagine-glycine pairs in SEQ ID NO: 25 and optionally further comprising subpopulations comprising other deamidated amino acids, wherein the deamidation results in an amino acid change; and (B) a vector genome in the AAV3B.AR2.14 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.14 capsid comprises: a heterogeneous population of vp1 proteins which are the product of a nucleic acid sequence encoding the amino acid sequence of SEQ ID NO: 25, a heterogeneous population of vp2 proteins which are the product of a nucleic acid sequence encoding the amino acid sequence of at least about amino acids 138 to 736 of SEQ ID NO: 25, and a heterogeneous population of vp3 proteins which are the product of a nucleic acid sequence encoding at least amino acids 203 to 736 of SEQ ID NO: 25.

In certain embodiments, the nucleic acid sequence encoding the AAV3B.AR2.14 vp1 capsid protein is provided in SEQ ID NO: 26. In other embodiments, a nucleic acid sequence of 70% to 99.9% identity to SEQ ID NO: 26 may be selected to express the AAV3B.AR2.14 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 26. However, other nucleic acid sequences which encode the amino acid sequence of SEQ ID NO: 25 may be selected for use in producing rAAV capsids. In certain embodiments, the nucleic acid sequence has the nucleic acid sequence of SEQ ID NO: 26 or a sequence at least 70% to 99% identical, at least 75%, at least 80%, at least 85%, at least 90%, at least 95%, at least 97%, at least 99%, identical to SEQ ID NO: 26 which encodes SEQ ID NO: 25. In certain embodiments, the nucleic acid sequence has the nucleic acid sequence of SEQ ID NO: 26 or a sequence at least 70% to 99%, at least 75%, at least 80%, at least 85%, at least 90%, at least 95%, at least 97%, at least 99%, identical to about nt 412 to about nt 2211 of SEQ ID NO: 26 which encodes the vp2 capsid protein (about aa 138 to 736) of SEQ ID NO: 25. In certain embodiments, the nucleic acid sequence has the nucleic acid sequence of about nt 607 to about nt 2211 of SEQ ID NO: 26 or a sequence at least 70% to 99%, at least 75%, at least 80%, at least 85%, at least 90%, at least 95%, at least 97%, at least 99%, identical to nt SEQ ID NO: 26 which encodes the vp3 capsid protein (about aa 203 to 736) of SEQ ID NO: 25.

The invention also encompasses nucleic acid sequences encoding mutant AAV3B.AR2.14, in which one or more residues has been altered in order to decrease deamidation, or other modifications which are identified herein. Such nucleic acid sequences can be used in production of mutant AAV3B.AR2.14 capsids.

In certain embodiments, a novel isolated AAV3B.AR2.15 capsid is provided. The nucleic acid sequence encoding the AAV is provided in SEQ ID NO: 28 and the predicted encoded amino acid sequence is provided in SEQ ID NO: 27. Provided herein is an rAAV comprising at least one of the vp1 (aa 1 to 736), vp2 (aa 138 to 736) and the vp3 (aa 203 to 736) of AAV3B.AR2.15 (SEQ ID NO: 27). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.15 (SEQ ID NO: 28).

In a further aspect, a recombinant adeno-associated virus (rAAV) is provided which comprises: (A) an AAV3B.AR2.15 capsid comprising one or more of: (1) AAV3B.AR2.15 capsid proteins comprising: a heterogeneous population of AAV3B.AR2.15 vp1 proteins selected from: vp1 proteins produced by expression from a nucleic acid sequence which encodes the predicted amino acid sequence of 1 to 736 of SEQ ID NO: 27, vp1 proteins produced from SEQ ID NO: 28, or vp1 proteins produced from a nucleic acid sequence at least 70% identical to SEQ ID NO: 28 which encodes the predicted amino acid sequence of 1 to 736 of SEQ ID NO: 27, a heterogeneous population of AAV3B.AR2.15 vp2 proteins selected from: vp2 proteins produced by expression from a nucleic acid sequence which encodes the predicted amino acid sequence of at least about amino acids 138 to 736 of SEQ ID NO: 27, vp2 proteins produced from a sequence comprising at least nucleotides 412 to 2211 of SEQ ID NO: 28, or vp2 proteins produced from a nucleic acid sequence at least 70% identical to at least nucleotides 412 to 2211 of SEQ ID NO: 28 which encodes the predicted amino acid sequence of at least about amino acids 138 to 736 of SEQ ID NO: 27, a heterogeneous population of AAV3B.AR2.15 vp3 proteins selected from: vp3 proteins produced by expression from a nucleic acid sequence which encodes the predicted amino acid sequence of at least about amino acids 203 to 736 of SEQ ID NO: 27, vp3 proteins produced from a sequence comprising at least nucleotides 607 to 2211 of SEQ ID NO: 28, or vp3 proteins produced from a nucleic acid sequence at least 70% identical to at least nucleotides 607 to 2211 of SEQ ID NO: 28 which encodes the predicted amino acid sequence of at least about amino acids 203 to 736 of SEQ ID NO: 27; and/or (2) a heterogeneous population of vp1 proteins which are the product of a nucleic acid sequence encoding the amino acid sequence of SEQ ID NO: 27, a heterogeneous population of vp2 proteins which are the product of a nucleic acid sequence encoding the amino acid sequence of at least about amino acids 138 to 736 of SEQ ID NO: 27, and a heterogeneous population of vp3 proteins which are the product of a nucleic acid sequence encoding at least amino acids 203 to 736 of SEQ ID NO: 27, wherein: the vp1, vp2 and vp3 proteins contain subpopulations with amino acid modifications comprising at least two highly deamidated asparagines (N) in asparagine-glycine pairs in SEQ ID NO: 27 and optionally further comprising subpopulations comprising other deamidated amino acids, wherein the deamidation results in an amino acid change; and (B) a vector genome in the AAV3B.AR2.15 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.15 capsid comprises: a heterogeneous population of vp1 proteins which are the product of a nucleic acid sequence encoding the amino acid sequence of SEQ ID NO: 27, a heterogeneous population of vp2 proteins which are the product of a nucleic acid sequence encoding the amino acid sequence of at least about amino acids 138 to 736 of SEQ ID NO: 27, and a heterogeneous population of vp3 proteins which are the product of a nucleic acid sequence encoding at least amino acids 203 to 736 of SEQ ID NO: 27.

In certain embodiments, the nucleic acid sequence encoding the AAV3B.AR2.15 vp1 capsid protein is provided in SEQ ID NO: 28. In other embodiments, a nucleic acid sequence of 70% to 99.9% identity to SEQ ID NO: 28 may be selected to express the AAV3B.AR2.15 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 28. However, other nucleic acid sequences which encode the amino acid sequence of SEQ ID NO: 27 may be selected for use in producing rAAV capsids. In certain embodiments, the nucleic acid sequence has the nucleic acid sequence of SEQ ID NO: 28 or a sequence at least 70% to 99% identical, at least 75%, at least 80%, at least 85%, at least 90%, at least 95%, at least 97%, at least 99%, identical to SEQ ID NO: 28 which encodes SEQ ID NO: 27. In certain embodiments, the nucleic acid sequence has the nucleic acid sequence of SEQ ID NO: 28 or a sequence at least 70% to 99%, at least 75%, at least 80%, at least 85%, at least 90%, at least 95%, at least 97%, at least 99%, identical to about nt 412 to about nt 2211 of SEQ ID NO: 28 which encodes the vp2 capsid protein (about aa 138 to 736) of SEQ ID NO: 27. In certain embodiments, the nucleic acid sequence has the nucleic acid sequence of about nt 607 to about nt 2211 of SEQ ID NO: 28 or a sequence at least 70% to 99%, at least 75%, at least 80%, at least 85%, at least 90%, at least 95%, at least 97%, at least 99%, identical to nt SEQ ID NO: 28 which encodes the vp3 capsid protein (about aa 203 to 736) of SEQ ID NO: 27.

The invention also encompasses nucleic acid sequences encoding mutant AAV3B.AR2.15, in which one or more residues has been altered in order to decrease deamidation, or other modifications which are identified herein. Such nucleic acid sequences can be used in production of mutant AAV3B.AR2.15 capsids.

In certain embodiments, a novel isolated AAV3B.AR2.16 capsid is provided. The nucleic acid sequence encoding the AAV is provided in SEQ ID NO: 30 and the predicted encoded amino acid sequence is provided in SEQ ID NO: 29. Provided herein is an rAAV comprising at least one of the vp1 (aa 1 to 736), vp2 (aa 138 to 736) and the vp3 (aa 203 to 736) of AAV3B.AR2.16 (SEQ ID NO: 29). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.16 (SEQ ID NO: 30).

In a further aspect, a recombinant adeno-associated virus (rAAV) is provided which comprises: (A) an AAV3B.AR2.16 capsid comprising one or more of: (1) AAV3B.AR2.16 capsid proteins comprising: a heterogeneous population of AAV3B.AR2.16 vp1 proteins selected from: vp1 proteins produced by expression from a nucleic acid sequence which encodes the predicted amino acid sequence of 1 to 736 of SEQ ID NO: 29, vp1 proteins produced from SEQ ID NO: 30, or vp1 proteins produced from a nucleic acid sequence at least 70% identical to SEQ ID NO: 30 which encodes the predicted amino acid sequence of 1 to 736 of SEQ ID NO: 29, a heterogeneous population of AAV3B.AR2.16 vp2 proteins selected from: vp2 proteins produced by expression from a nucleic acid sequence which encodes the predicted amino acid sequence of at least about amino acids 138 to 736 of SEQ ID NO: 29, vp2 proteins produced from a sequence comprising at least nucleotides 412 to 2211 of SEQ ID NO: 30, or vp2 proteins produced from a nucleic acid sequence at least 70% identical to at least nucleotides 412 to 2211 of SEQ ID NO: 30 which encodes the predicted amino acid sequence of at least about amino acids 138 to 736 of SEQ ID NO: 29, a heterogeneous population of AAV3B.AR2.16 vp3 proteins selected from: vp3 proteins produced by expression from a nucleic acid sequence which encodes the predicted amino acid sequence of at least about amino acids 203 to 736 of SEQ ID NO: 29, vp3 proteins produced from a sequence comprising at least nucleotides 607 to 2211 of SEQ ID NO: 30, or vp3 proteins produced from a nucleic acid sequence at least 70% identical to at least nucleotides 607 to 2211 of SEQ ID NO: 30 which encodes the predicted amino acid sequence of at least about amino acids 203 to 736 of SEQ ID NO: 29; and/or (2) a heterogeneous population of vp1 proteins which are the product of a nucleic acid sequence encoding the amino acid sequence of SEQ ID NO: 29, a heterogeneous population of vp2 proteins which are the product of a nucleic acid sequence encoding the amino acid sequence of at least about amino acids 138 to 736 of SEQ ID NO: 29, and a heterogeneous population of vp3 proteins which are the product of a nucleic acid sequence encoding at least amino acids 203 to 736 of SEQ ID NO: 29, wherein: the vp1, vp2 and vp3 proteins contain subpopulations with amino acid modifications comprising at least two highly deamidated asparagines (N) in asparagine-glycine pairs in SEQ ID NO: 29 and optionally further comprising subpopulations comprising other deamidated amino acids, wherein the deamidation results in an amino acid change; and (B) a vector genome in the AAV3B.AR2.16 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.16 capsid comprises: a heterogeneous population of vp1 proteins which are the product of a nucleic acid sequence encoding the amino acid sequence of SEQ ID NO: 29, a heterogeneous population of vp2 proteins which are the product of a nucleic acid sequence encoding the amino acid sequence of at least about amino acids 138 to 736 of SEQ ID NO: 29, and a heterogeneous population of vp3 proteins which are the product of a nucleic acid sequence encoding at least amino acids 203 to 736 of SEQ ID NO: 29.

In certain embodiments, the nucleic acid sequence encoding the AAV3B.AR2.16 vp1 capsid protein is provided in SEQ ID NO: 30. In other embodiments, a nucleic acid sequence of 70% to 99.9% identity to SEQ ID NO: 30 may be selected to express the AAV3B.AR2.16 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 30. However, other nucleic acid sequences which encode the amino acid sequence of SEQ ID NO: 29 may be selected for use in producing rAAV capsids. In certain embodiments, the nucleic acid sequence has the nucleic acid sequence of SEQ ID NO: 30 or a sequence at least 70% to 99% identical, at least 75%, at least 80%, at least 85%, at least 90%, at least 95%, at least 97%, at least 99%, identical to SEQ ID NO: 30 which encodes SEQ ID NO: 29. In certain embodiments, the nucleic acid sequence has the nucleic acid sequence of SEQ ID NO: 30 or a sequence at least 70% to 99%, at least 75%, at least 80%, at least 85%, at least 90%, at least 95%, at least 97%, at least 99%, identical to about nt 412 to about nt 2211 of SEQ ID NO: 30 which encodes the vp2 capsid protein (about aa 138 to 736) of SEQ ID NO: 29. In certain embodiments, the nucleic acid sequence has the nucleic acid sequence of about nt 607 to about nt 2211 of SEQ ID NO: 30 or a sequence at least 70% to 99%, at least 75%, at least 80%, at least 85%, at least 90%, at least 95%, at least 97%, at least 99%, identical to nt SEQ ID NO: 30 which encodes the vp3 capsid protein (about aa 203 to 736) of SEQ ID NO: 29.

The invention also encompasses nucleic acid sequences encoding mutant AAV3B.AR2.16, in which one or more residues has been altered in order to decrease deamidation, or other modifications which are identified herein. Such nucleic acid sequences can be used in production of mutant AAV3B.AR2.16 capsids.

In certain embodiments, a novel isolated AAV3B.AR2.17 capsid is provided. The nucleic acid sequence encoding the AAV is provided in SEQ ID NO: 32 and the predicted encoded amino acid sequence is provided in SEQ ID NO: 31. Provided herein is an rAAV comprising at least one of the vp1 (aa 1 to 736), vp2 (aa 138 to 736) and the vp3 (aa 203 to 736) of AAV3B.AR2.17 (SEQ ID NO: 31). Also provided herein are rAAV comprising an AAV capsid encoded by at least one of the vp1 (nt 1 to nt 2211), vp2 (nt 412 to nt 2211) and the vp3 (nt 607 to nt 2211) of AAV3B.AR2.17 (SEQ ID NO: 32).

In a further aspect, a recombinant adeno-associated virus (rAAV) is provided which comprises: (A) an AAV3B.AR2.17 capsid comprising one or more of: (1) AAV3B.AR2.17 capsid proteins comprising: a heterogeneous population of AAV3B.AR2.17 vp1 proteins selected from: vp1 proteins produced by expression from a nucleic acid sequence which encodes the predicted amino acid sequence of 1 to 736 of SEQ ID NO: 31, vp1 proteins produced from SEQ ID NO: 32, or vp1 proteins produced from a nucleic acid sequence at least 70% identical to SEQ ID NO: 32 which encodes the predicted amino acid sequence of 1 to 736 of SEQ ID NO: 31, a heterogeneous population of AAV3B.AR2.17 vp2 proteins selected from: vp2 proteins produced by expression from a nucleic acid sequence which encodes the predicted amino acid sequence of at least about amino acids 138 to 736 of SEQ ID NO: 31, vp2 proteins produced from a sequence comprising at least nucleotides 412 to 2211 of SEQ ID NO: 32, or vp2 proteins produced from a nucleic acid sequence at least 70% identical to at least nucleotides 412 to 2211 of SEQ ID NO: 32 which encodes the predicted amino acid sequence of at least about amino acids 138 to 736 of SEQ ID NO: 31, a heterogeneous population of AAV3B.AR2.17 vp3 proteins selected from: vp3 proteins produced by expression from a nucleic acid sequence which encodes the predicted amino acid sequence of at least about amino acids 203 to 736 of SEQ ID NO: 31, vp3 proteins produced from a sequence comprising at least nucleotides 607 to 2211 of SEQ ID NO: 32, or vp3 proteins produced from a nucleic acid sequence at least 70% identical to at least nucleotides 607 to 2211 of SEQ ID NO: 32 which encodes the predicted amino acid sequence of at least about amino acids 203 to 736 of SEQ ID NO: 31; and/or (2) a heterogeneous population of vp1 proteins which are the product of a nucleic acid sequence encoding the amino acid sequence of SEQ ID NO: 31, a heterogeneous population of vp2 proteins which are the product of a nucleic acid sequence encoding the amino acid sequence of at least about amino acids 138 to 736 of SEQ ID NO: 31, and a heterogeneous population of vp3 proteins which are the product of a nucleic acid sequence encoding at least amino acids 203 to 736 of SEQ ID NO: 31, wherein: the vp1, vp2 and vp3 proteins contain subpopulations with amino acid modifications comprising at least two highly deamidated asparagines (N) in asparagine-glycine pairs in SEQ ID NO: 31 and optionally further comprising subpopulations comprising other deamidated amino acids, wherein the deamidation results in an amino acid change; and (B) a vector genome in the AAV3B.AR2.17 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, an AAV3B.AR2.17 capsid comprises: a heterogeneous population of vp1 proteins which are the product of a nucleic acid sequence encoding the amino acid sequence of SEQ ID NO: 31, a heterogeneous population of vp2 proteins which are the product of a nucleic acid sequence encoding the amino acid sequence of at least about amino acids 138 to 736 of SEQ ID NO: 31, and a heterogeneous population of vp3 proteins which are the product of a nucleic acid sequence encoding at least amino acids 203 to 736 of SEQ ID NO: 31.

In certain embodiments, the nucleic acid sequence encoding the AAV3B.AR2.17 vp1 capsid protein is provided in SEQ ID NO: 32. In other embodiments, a nucleic acid sequence of 70% to 99.9% identity to SEQ ID NO: 32 may be selected to express the AAV3B.AR2.17 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 32. However, other nucleic acid sequences which encode the amino acid sequence of SEQ ID NO: 31 may be selected for use in producing rAAV capsids. In certain embodiments, the nucleic acid sequence has the nucleic acid sequence of SEQ ID NO: 32 or a sequence at least 70% to 99% identical, at least 75%, at least 80%, at least 85%, at least 90%, at least 95%, at least 97%, at least 99%, identical to SEQ ID NO: 32 which encodes SEQ ID NO: 31. In certain embodiments, the nucleic acid sequence has the nucleic acid sequence of SEQ ID NO: 32 or a sequence at least 70% to 99%, at least 75%, at least 80%, at least 85%, at least 90%, at least 95%, at least 97%, at least 99%, identical to about nt 412 to about nt 2211 of SEQ ID NO: 32 which encodes the vp2 capsid protein (about aa 138 to 736) of SEQ ID NO: 31. In certain embodiments, the nucleic acid sequence has the nucleic acid sequence of about nt 607 to about nt 2211 of SEQ ID NO: 32 or a sequence at least 70% to 99%, at least 75%, at least 80%, at least 85%, at least 90%, at least 95%, at least 97%, at least 99%, identical to nt SEQ ID NO: 32 which encodes the vp3 capsid protein (about aa 203 to 736) of SEQ ID NO: 31.

The invention also encompasses nucleic acid sequences encoding mutant AAV3B.AR2.17, in which one or more residues has been altered in order to decrease deamidation, or other modifications which are identified herein. Such nucleic acid sequences can be used in production of mutant AAV3B.AR2.17 capsids.

### B. rAAV Vectors and Compositions

In another aspect, described herein are molecules which utilize the AAV capsid sequences described herein, including fragments thereof, for production of viral vectors useful in delivery of a heterologous gene or other nucleic acid sequences to a target cell. In certain embodiments, the vectors useful in compositions and methods described herein contain, at a minimum, sequences encoding a selected AAV capsid as described herein, e.g., an AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.08 (SEQ ID NO: 15), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), AAV3B.AR2.16 (SEQ ID NO: 29), or AAV3B.AR2.17 (SEQ ID NO: 31) capsid, or a fragment thereof, including the vp1, vp2, or vp3 capsid protein. In another embodiment, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 origin. Alternatively, vectors may be used in which the rep sequences are from an AAV which differs from the wild type AAV providing the cap sequences, e.g., the same AAV providing the ITRs and rep.

In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein. Optionally, the vectors further contain a minigene comprising a selected transgene which is flanked by AAV 5′ ITR and AAV 3′ ITR. In another embodiment, the AAV is a self-complementary AAV (sc-AAV) (See, U.S. 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to ˜55 kd) and any currently available RNA-based therapy.

Pseudotyped vectors, wherein the capsid of one AAV is replaced with a heterologous capsid protein, are useful herein. For illustrative purposes, AAV vectors utilizing an AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 capsid as described herein, with AAV2 ITRs are used in the examples described below. See, Mussolino et al, cited above. Unless otherwise specified, the AAV ITRs, and other selected AAV components described herein, may be individually selected from among any AAV serotype, including, without limitation, AAV1, AAV2, AAV3, AAV4, AAV5, AAV6, AAV7, AAV8, AAV9 or other known and unknown AAV serotypes. In one desirable embodiment, the ITRs of AAV serotype 2 are used. However, ITRs from other suitable serotypes may be selected. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The rAAV described herein also comprise a vector genome. The vector genome is composed of, at a minimum, a non-AAV or heterologous nucleic acid sequence (the transgene), as described below, and its regulatory sequences, and 5′ and 3′ AAV inverted terminal repeats (ITRs). It is this minigene which is packaged into a capsid protein and delivered to a selected target cell.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The AAV may comprise one or more transgenes.

Therapeutic Transgenes

Useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In most embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgi-bin/Disease_Search_List.php; rarediseases.info.nih.gov/diseases. In one embodiment, the transgene is not human low-density lipoprotein receptor (hLDLR). In another embodiment, the transgene is not an engineered human low-density lipoprotein receptor (hLDLR) variant, such as those described in WO 2015/164778.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-IL33, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include hydroxymethylbilane synthase (HMBS), carbamoyl synthetase I, ornithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), chorionic gonadotrophin (CG), glucose-6-phosphatase, porphobilinogen deaminase, cystathionine beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase E3A (UBE3A). Still useful gene products include UDP Glucuronosyltransferase Family 1 Member A1 (UGT1A1).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Other useful gene products include non-naturally occurring polypeptides, such as chimeric or hybrid polypeptides having a non-naturally occurring amino acid sequence containing insertions, deletions, or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Further illustrative genes which may be delivered via the rAAV provided herein for treatment of, for example, liver indications include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbarnylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO2015/164778; PCSK9; ApoE and ApoC proteins, associated with dementia; UDP-glucuronosyltransferase, associated with Crigler-Najjar disease; adenosine deaminase, associated with severe combined immunodeficiency disease; hypoxanthine guanine phosphoribosyl transferase, associated with Gout and Lesch-Nyhan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Additional genes and diseases of interest include, e.g., dystonin gene related diseases such as Hereditary Sensory and Autonomic Neuropathy Type VI (the DST gene encodes dystonin; dual AAV vectors may be required due to the size of the protein (˜7570 aa); SCN9A related diseases, in which loss of function mutants cause inability to feel pain and gain of function mutants cause pain conditions, such as erythromelalgia. Another condition is Charcot-Marie-Tooth (CMT) type 1F and 2E due to mutations in the NEFL gene (neurofilament light chain) characterized by a progressive peripheral motor and sensory neuropathy with variable clinical and electrophysiologic expression. Other gene products associated with CMT include mitofusin 2 (MFN2).

In certain embodiments, the rAAV described herein may be used in treatment of mucopolysaccharidoses (MPS) disorders. Such rAAV may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome).

Immunogenic Transgenes

In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelin phosphodiesterase 1 (SMPD1), SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBI, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the invention: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-00*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518e*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520c, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-mi-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. For example, miRNA targeting chromosome 8 open reading frame 72 (C9orf72) which expresses superoxide dismutase (SOD1), associated with amyotrophic lateral sclerosis (ALS) may be of interest.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA, As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex.

Still other useful transgenes may include those encoding immunoglobulins which confer passive immunity to a pathogen. An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The anti-pathogen construct is selected based on the causative agent (pathogen) for the disease against which protection is sought. These pathogens may be of viral, bacterial, or fungal origin, and may be used to prevent infection in humans against human disease, or in non-human mammals or other animals to prevent veterinary disease.

The rAAV may include genes encoding antibodies, and particularly neutralizing antibodies against a viral pathogen. Such anti-viral antibodies may include anti-influenza antibodies directed against one or more of Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Other target pathogenic viruses include, arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory syncitial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease. Thus, in certain embodiments, a rAAV vector as described herein may be engineered to express an anti-ebola antibody, e.g., 2G4, 4G7, 13C6, an anti-influenza antibody, e.g., FI6, CR8033, and anti-RSV antibody, e.g, palivizumab, motavizumab. A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The rAAV may include genes encoding antibodies, and particularly neutralizing antibodies against a bacterial pathogen such as the causative agent of anthrax, a toxin produced by Bacillus anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

The rAAV may include genes encoding antibodies, and particularly neutralizing antibodies, against pathogenic factors of diseases such as Alzheimer's disease (AD), Parkinson's disease (PD), GBA-associated-Parkinson's disease (GBA-PD), Rheumatoid arthritis (RA), Irritable bowel syndrome (IBS), chronic obstructive pulmonary disease (COPD), cancers, tumors, systemic sclerosis, asthma and other diseases. Such antibodies may be, without limitation, e.g., alpha-synuclein, anti-vascular endothelial growth factor (VEGF) (anti-VEGF), anti-VEGFA, anti-PD-1, anti-PDL1, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-ibeta, anti-epidermal growth factor receptor, anti-CD25, anti-RANK ligand, anti-complement system protein C5, anti-CD11a, anti-CD3 receptor, anti-alpha-4 (α4) integrin, anti-RSV F protein, and anti-integrin α₄β₇. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-beta-amyloid fibril, anti-beta-amyloid plaques, anti-tau, a bapineuzumab, among others. Other suitable antibodies for treating a variety of indications include those described, e.g., in PCT/US2016/058968, filed 27 Oct. 2016, published as WO 2017/075119A1.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Alternatively, or in addition, the vectors may contain AAV sequences of the invention and a transgene encoding a peptide, polypeptide or protein which induces an immune response to a selected immunogen. For example, immunogens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutinin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentiviral (which includes human immunodeficiency virus (HIV), simian immunodeficiency virus (SIV), feline immunodeficiency virus (FIV), equine infectious anemia virus, and spumavirinal). Between the HIV and SIV, many suitable antigens have been described and can readily be selected. Examples of suitable HIV and SIV antigens include, without limitation the gag, pol, Vif, Vpx, VPR, Env, Tat and Rev proteins, as well as various fragments thereof. In addition, a variety of modifications to these antigens have been described. Suitable antigens for this purpose are known to those of skill in the art. For example, one may select a sequence encoding the gag, pol, Vif, and Vpr, Env, Tat and Rev, amongst other proteins. See, e.g., the modified gag protein which is described in U.S. Pat. No. 5,972,596. See, also, the HIV and SIV proteins described in D. H. Barouch et al, J. Virol., 75(5):2462-2467 (March 2001), and R. R. Amara, et al, Science, 292:69-74 (6 Apr. 2001). These proteins or subunits thereof may be delivered alone, or in combination via separate vectors or from a single vector.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; meliodosis; salmonella; shigella; haemophilus; moraxella; H. ducreyi (which causes chancroid); brucella; Francisella tularensis (which causes tularemia); yersinia (pasteurella); Streptobacillus moniliformis and spirillum; Gram-positive bacilli include Listeria monocytogenes; Erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), Variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricinus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of T cell receptors (TCRs) which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a nucleic acid molecule that encodes at least one of these polypeptides will elicit an immune response that will target T cells involved in scleroderma.

In one embodiment, the transgene is selected to provide optogenetic therapy. In optogenetic therapy, artificial photoreceptors are constructed by gene delivery of light-activated channels or pumps to surviving cell types in the remaining retinal circuit. This is particularly useful for patients who have lost a significant amount of photoreceptor function, but whose bipolar cell circuitry to ganglion cells and optic nerve remains intact. In one embodiment, the heterologous nucleic acid sequence (transgene) is an opsin. The opsin sequence can be derived from any suitable single- or multicellular-organism, including human, algae and bacteria. In one embodiment, the opsin is rhodopsin, photopsin, L/M wavelength (red/green) -opsin, or short wavelength (S) opsin (blue). In another embodiment, the opsin is channelrhodopsin or halorhodopsin.

In another embodiment, the transgene is selected for use in gene augmentation therapy, i.e., to provide replacement copy of a gene that is missing or defective. In this embodiment, the transgene may be readily selected by one of skill in the art to provide the necessary replacement gene. In one embodiment, the missing/defective gene is related to an ocular disorder. In another embodiment, the transgene is NYX, GRM6, TRPM1L or GPR179 and the ocular disorder is Congenital Stationary Night Blindness. See, e.g., Zeitz et al, Am J Hum Genet. 2013 Jan. 10; 92(1):67-75. Epub 2012 Dec. 13 which is incorporated herein by reference. In another embodiment, the transgene is RPGR. In another embodiment, the gene is Rab escort protein 1 (REP-1) encoded by CHM, associated with choroideremia.

In another embodiment, the transgene is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126(3):764-74 and O'Reilly M, et al. Am J Hum Genet. 2007 July; 81(1):127-35, which are incorporated herein by reference. In this embodiment, the transgene may be readily selected by one of skill in the art based upon the gene which is desired to be silenced.

In another embodiment, the transgene comprises more than one transgene. This may be accomplished using a single vector carrying two or more heterologous sequences, or using two or more rAAV each carrying one or more heterologous sequences. In one embodiment, the rAAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. In one embodiment, the transgene encodes a nuclease selected from a meganuclease, a zinc finger nuclease, a transcription activator-like (TAL) effector nuclease (TALEN), and a clustered, regularly interspaced short palindromic repeat (CRISPR)/endonuclease (Cas9, Cpf1, etc). Examples of suitable meganucleases are described, e.g., in U.S. Pat. Nos. 8,445,251; 9,340,777; 9,434,931; 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (January 2020)). In certain embodiments, the nuclease is not a zinc finger nuclease. In certain embodiments, the nuclease is not a CRISPR-associated nuclease. In certain embodiments, the nuclease is not a TALEN. In one embodiment, the nuclease is not a meganuclease. In certain embodiments, the nuclease is a member of the LAGLIDADG (SEQ ID NO: 45) family of homing endonucleases. In certain embodiments, the nuclease is a member of the I-CreI family of homing endonucleases which recognizes and cuts a 22 base pair recognition sequence SEQ ID NO: 46-CAAAACGTCGTGAGACAGTTTG. See, e.g., WO 2009/059195. Methods for rationally-designing mono-LAGLIDADG homing endonucleases were described which are capable of comprehensively redesigning ICreI and other homing endonucleases to target widely-divergent DNA sites, including sites in mammalian, yeast, plant, bacterial, and viral genomes (WO 2007/047859).

Suitable gene editing targets include, e.g., liver-expressed genes such as, without limitation, proprotein convertase subtilisin/kexin type 9 (PCSK9) (cholesterol related disorders), transthyretin (TTR) (transthyretin amyloidosis), HAO, apolipoprotein C-III (APOC3), Factor VIII, Factor IX, low density lipoprotein receptor (LDLr), lipoprotein lipase (LPL) (Lipoprotein Lipase Deficiency), lecithin-cholesterol acyltransferase (LCAT), ornithine transcarbamylase (OTC), carnosinase (CN1), sphingomyelin phosphodiesterase (SMPD1) (Niemann-Pick disease), hypoxanthine-guanine phosphoribosyltransferase (HGPRT), branched-chain alpha-keto acid dehydrogenase complex (BCKDC) (maple syrup urine disease), erythropoietin (EPO), Carbamyl Phosphate Synthetase (CPS1), N-Acetylglutamate Synthetase (NAGS), Argininosuccinic Acid Synthetase (Citrullinemia), Argininosuccinate Lyase (ASL) (Argininosuccinic Aciduria), and Arginase (AG).

Other gene editing targets may include, e.g., hydroxymethylbilane synthase (HMBS), carbamoyl synthetase I, ornithine transcarbamylase (OTC), arginosuccinate synthetase, alpha 1 anti-trypsin (A1AT), aaporginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathionine beta-synthase, branched chain ketoacid decarboxylase, albumin, isovaleryl-coA dehydrogenase, propionyl CoA carboxylase, methyl malonyl CoA mutase (MUT), glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase. glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-Ela, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucuronosyltransferase, associated with Crigler-Najjar disease; adenosine deaminase, associated with severe combined immunodeficiency disease; hypoxanthine guanine phosphoribosyl transferase, associated with Gout and Lesch-Nyhan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In one embodiment, the capsids described herein are useful in the CRISPR-Cas dual vector system described in U.S. Published Patent Application 2018/0110877, filed Apr. 26, 2018, each of which is incorporated herein by reference. The capsids are also useful for delivery homing endonucleases or other meganucleases.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, in addition to the transgene coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

These above coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

Desirably, the transgene encodes a product which is useful in biology and medicine, such as proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated animal.

The regulatory sequences include conventional control elements which are operably linked to the transgene in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In one embodiment, the AAV comprises a promoter (or a functional fragment of a promoter). The selection of the promoter to be employed in the rAAV may be made from among a wide number of constitutive or inducible promoters that can express the selected transgene in the desired target cell. In one embodiment, the target cell is an ocular cell. The promoter may be derived from any species, including human. Desirably, in one embodiment, the promoter is “cell specific”. The term “cell-specific” means that the particular promoter selected for the recombinant vector can direct expression of the selected transgene in a particular cell tissue. In one embodiment, the promoter is specific for expression of the transgene in muscle cells. In another embodiment, the promoter is specific for expression in lung. In another embodiment, the promoter is specific for expression of the transgene in liver cells. In another embodiment, the promoter is specific for expression of the transgene in airway epithelium. In another embodiment, the promoter is specific for expression of the transgene in neurons. In another embodiment, the promoter is specific for expression of the transgene in heart.

The expression cassette typically contains a promoter sequence as part of the expression control sequences, e.g., located between the selected 5′ ITR sequence and the immunoglobulin construct coding sequence. In one embodiment, expression in liver is desirable. Thus, in one embodiment, a liver-specific promoter is used. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14). Tissue specific promoters, constitutive promoters, regulatable promoters [see, e.g., WO 2011/126808 and WO 2013/04943], or a promoter responsive to physiologic cues may be used may be utilized in the vectors described herein. In another embodiment, expression in muscle is desirable. Thus, in one embodiment, a muscle-specific promoter is used. In one embodiment, the promoter is an MCK based promoter, such as the dMCK (509-bp) or tMCK (720-bp) promoters (see, e.g., Wang et al, Gene Ther. 2008 November; 15(22):1489-99. doi: 10.1038/gt.2008.104. Epub 2008 Jun. 19, which is incorporated herein by reference). Another useful promoter is the SPc5-12 promoter (see Rasowo et al, European Scientific Journal June 2014 edition vol. 10, No. 18, which is incorporated herein by reference). In certain embodiments, a promoter specific for the eye or a subpart thereof (e.g., retina) may be selected.

In one embodiment, the promoter is a CMV promoter. In another embodiment, the promoter is a TBG promoter. In another embodiment, a CB7 promoter is used. CB7 is a chicken β-actin promoter with cytomegalovirus enhancer elements. Alternatively, other liver-specific promoters may be used [see, e.g., The Liver Specific Gene Promoter Database, Cold Spring Harbor, rulai.schl.edu/LSPD, alpha 1 anti-trypsin (A1AT); human albumin Miyatake et al., J. Virol., 71:5124 32 (1997), humAlb; and hepatitis B virus core promoter, Sandig et al., Gene Ther., 3:1002 9 (1996)]. TTR minimal enhancer/promoter, alpha-antitrypsin promoter, LSP (845 nt) 25 (requires intron-less scAAV).

The promoter(s) can be selected from different sources, e.g., human cytomegalovirus (CMV) immediate-early enhancer/promoter, the SV40 early enhancer/promoter, the JC polyomavirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The expression cassette may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter-enhancer elements may be selected. Other enhancer sequences useful herein include the IRBP enhancer (Nicoud 2007, J Gene Med. 2007 December; 9(12):1015-23), immediate early cytomegalovirus enhancer, one derived from an immunoglobulin gene or SV40 enhancer, the cis-acting element identified in the mouse proximal promoter, etc.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A variety of suitable polyA are known. In one example, the polyA is rabbit beta globin, such as the 127 bp rabbit beta-globin polyadenylation signal (GenBank #V00882.1). In other embodiments, an SV40 polyA signal is selected. Still other suitable polyA sequences may be selected. In certain embodiments, an intron is included. One suitable intron is a chicken beta-actin intron. In one embodiment, the intron is 875 bp (GenBank #X00182.1). In another embodiment, a chimeric intron available from Promega is used. However, other suitable introns may be selected. In one embodiment, spacers are included such that the vector genome is approximately the same size as the native AAV vector genome (e.g., between 4.1 and 5.2 kb). In one embodiment, spacers are included such that the vector genome is approximately 4.7 kb. See, Wu et al, Effect of Genome Size on AAV Vector Packaging, Mol Ther. 2010 January; 18(1): 80-86, which is incorporated herein by reference.

In certain embodiments, the expression cassette further comprises dorsal root ganglion (drg)-specific miRNA detargeting sequences operably linked to the transgene coding sequence. In certain embodiments, the tandem miRNA target sequences are continuous or are separated by a spacer of 1 to 10 nucleic acids, wherein said spacer is not an miRNA target sequence. In certain embodiments, there are at least two drg-specific miRNA sequences located at 3′ to a functional transgene coding sequence. In certain embodiments, the start of the first of the at least two drg-specific miRNA tandem repeats is within 20 nucleotides from the 3′ end of the transgene coding sequence. In certain embodiments, the start of the first of the at least two drg-specific miRNA tandem repeats is at least 100 nucleotides from the 3′ end of the functional transgene coding sequence. In certain embodiments, the miRNA tandem repeats comprise 200 to 1200 nucleotides in length. In certain embodiments, there are at least two drg-specific miRNA target sequences located at 5′ to the functional transgene coding sequence. In certain embodiments, at least two drg-specific miRNA target sequences are located in both 5′ and 3′ to the functional transgene coding sequence. In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 41); (ii) AGCAAAAATGTGCTAGTGCCAAA (SEQ ID NO: 42), (iii) AGTGTGAGTTCTACCATTGCCAAA (SEQ ID NO: 43); or (iv) AGGGATTCCTGGGAAAACTGGAC (SEQ ID NO: 44). In certain embodiments, the miRNA target sequence for the at least first and/or at least second miRNA target sequence for the expression cassette mRNA or DNA positive strand is AGTGAATTCTACCAGTGCCATA (miR183, SEQ ID NO: 41). In certain embodiments, the miRNA target sequence for the at least first and/or at least second miRNA target sequence for the expression cassette mRNA or DNA positive strand is AGTGAATTCTACCAGTGCCATA (miR182, SEQ ID NO: 42). In certain embodiments, two or more consecutive miRNA target sequences are continuous and not separated by a spacer. In certain embodiments, two or more of the miRNA target sequences are separated by a spacer and each spacer is independently selected from one or more of (A) GGAT; (B) CACGTG; or (C) GCATGC. In certain embodiments, the spacer located between the miRNA target sequences may be located 3′ to the first miRNA target sequence and/or 5′ to the last miRNA target sequence. In certain embodiments, the spacers between the miRNA target sequences are the same. See International Patent Application No. PCT/US19/67872, filed Dec. 20, 2019, U.S. Provisional Patent Application No. 63/023,594, filed May 12, 2020, U.S. Provisional Patent Application No. 63/038,488, filed Jun. 12, 2020, U.S. Provisional Patent Application No. 63/043,562, filed Jun. 24, 2020, and U.S. Provisional Patent Application No. 63/079,299, filed Sep. 16, 2020, all of which are incorporated by reference in their entireties.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989. Of course, not all vectors and expression control sequences will function equally well to express all of the transgenes as described herein. However, one of skill in the art may make a selection among these, and other, expression control sequences without departing from the scope of this invention.

In another embodiment, a method of generating a recombinant adeno-associated virus is provided. A suitable recombinant adeno-associated virus (AAV) is generated by culturing a host cell which contains a nucleic acid sequence encoding an AAV capsid protein as described herein, or fragment thereof, a functional rep gene; a minigene composed of, at a minimum, AAV inverted terminal repeats (ITRs) and a heterologous nucleic acid sequence encoding a desirable transgene; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Also provided herein are host cells transfected with an AAV as described herein. Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art. In another embodiment, the host cell comprises a nucleic acid molecule as described herein.

The minigene, rep sequences, cap sequences, and helper functions required for producing the rAAV described herein may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, 1993 J. Virol., 70:520-532 and U.S. Pat. No. 5,478,745, among others. These publications are incorporated by reference herein.

Also provided herein, are plasmids for use in producing the vectors described herein. Such plasmids are described in the Examples section.

### C. Pharmaceutical Compositions and Administration

In one embodiment, the recombinant AAV containing the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). The vector is formulated in a buffer/carrier suitable for infusion in human subjects. The buffer/carrier should include a component that prevents the rAAV from sticking to the infusion tubing but does not interfere with the rAAV binding activity in vivo.

In certain embodiments of the methods described herein, the pharmaceutical composition described above is administered to the subject intramuscularly. In other embodiments, the pharmaceutical composition is administered by intravenously. In other embodiments, the pharmaceutical composition is administered by intracerebroventricular injection. Other forms of administration that may be useful in the methods described herein include, but are not limited to, direct delivery to a desired organ (e.g., the eye, liver), including subretinal or intravitreal delivery, oral, inhalation, intranasal, intratracheal, intravenous, intramuscular, subcutaneous, intradermal, and other parental routes of administration. Routes of administration may be combined, if desired.

The composition may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

An effective concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)). In one embodiment, the rAAV vector genomes are measured by real-time PCR. In another embodiment, the rAAV vector genomes are measured by digital PCR. See, Lock et al, Absolute determination of single-stranded and self-complementary adeno-associated viral vector genome titers by droplet digital PCR, Hum Gene Ther Methods. 2014 April; 25 (2):115-25. doi: 10.1089/hgtb.2013.131. Epub 2014 Feb. 14, which are incorporated herein by reference. In another embodiment, the rAAV infectious units are measured as described in S. K. McLaughlin et al, 1988 J. Virol., 62:1963, which is incorporated herein by reference.

Preferably, the concentration is from about 1.5×10⁹ vg/mL to about 1.5×10¹³ vg/mL, and more preferably from about 1.5×10⁹ vg/mL to about 1.5×10¹¹ vg/mL. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. All ranges described herein are inclusive of the endpoints.

In one embodiment, the dosage is from about 1.5×10⁹ vg/kg of body weight to about 1.5×10¹³ vg/kg, and more preferably from about 1.5×10⁹ vg/kg to about 1.5×10¹¹ vg/kg. In one embodiment, the dosage is about 1.4×10⁸ vg/kg. In one embodiment, the dosage is about 3.5×10¹⁰ vg/kg. In another embodiment, the dosage is about 5.6×10¹¹ vg/kg. In another embodiment, the dosage is about 5.3×10¹² vg/kg. In yet another embodiment, the dosage is about 1.5×10¹² vg/kg. In another embodiment, the dosage is about 1.5×10¹³ vg/kg. In another embodiment, the dosage is about 3.0×10¹³ vg/kg. In another embodiment, the dosage is about 1.0×10¹⁴ vg/kg. All ranges described herein are inclusive of the endpoints.

In one embodiment, the effective dosage (total genome copies delivered) is from about 10⁷ to 10¹³ vector genomes. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed. Intravenous delivery, for example may require doses on the order of 1.5×10¹³ vg/kg.

### D. Methods

In another aspect, a method of transducing a target cell or tissue is provided. In one embodiment, the method includes administering an AAV having an AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 capsid as described herein. As shown in the examples below, the inventors have shown that the AAV3B mutants described herein effectively transduce liver, heart and muscle tissue. Therefore, provided herein is a method of transducing liver comprising administering an rAAV having the AAV3B.AR2.01 capsid. In another embodiment, provided herein is a method of transducing liver comprising administering an rAAV having the AAV3B.AR2.02 capsid. In another embodiment, provided herein is a method of transducing liver comprising administering an rAAV having the AAV3B.AR2.03 capsid. In another embodiment, provided herein is a method of transducing liver comprising administering an rAAV having the AAV3B.AR2.04 capsid. In another embodiment, provided herein is a method of transducing liver comprising administering an rAAV having the AAV3B.AR2.05 capsid. In another embodiment, provided herein is a method of transducing liver comprising administering an rAAV having the AAV3B.AR2.06 capsid. In another embodiment, provided herein is a method of transducing liver comprising administering an rAAV having the AAV3B.AR2.07 capsid. In another embodiment, provided herein is a method of transducing liver comprising administering an rAAV having the AAV3B.AR2.08 capsid. In another embodiment, provided herein is a method of transducing liver comprising administering an rAAV having the AAV3B.AR2.10 capsid. In another embodiment, provided herein is a method of transducing liver comprising administering an rAAV having the AAV3B.AR2.11 capsid. In another embodiment, provided herein is a method of transducing liver comprising administering an rAAV having the AAV3B.AR2.12 capsid. In another embodiment, provided herein is a method of transducing liver comprising administering an rAAV having the AAV3B.AR2.13 capsid. In another embodiment, provided herein is a method of transducing liver comprising administering an rAAV having the AAV3B.AR2.14 capsid. In another embodiment, provided herein is a method of transducing liver comprising administering an rAAV having the AAV3B.AR2.15 capsid. In another embodiment, provided herein is a method of transducing liver comprising administering an rAAV having the AAV3B.AR2.16 capsid. In another embodiment, provided herein is a method of transducing liver comprising administering an rAAV having the AAV3B.AR2.17 capsid. In one embodiment, intravenous administration is employed. In another embodiment, ICV administration is employed.

Also provided herein is a method of transducing heart comprising administering an rAAV having the AAV3B.AR2.01 capsid. In another embodiment, provided herein is a method of transducing heart comprising administering an rAAV having the AAV3B.AR2.02 capsid. In another embodiment, provided herein is a method of transducing heart comprising administering an rAAV having the AAV3B.AR2.03 capsid. In another embodiment, provided herein is a method of transducing heart comprising administering an rAAV having the AAV3B.AR2.04 capsid. In another embodiment, provided herein is a method of transducing heart comprising administering an rAAV having the AAV3B.AR2.05 capsid. In another embodiment, provided herein is a method of transducing heart comprising administering an rAAV having the AAV3B.AR2.06 capsid. In another embodiment, provided herein is a method of transducing heart comprising administering an rAAV having the AAV3B.AR2.07 capsid. In another embodiment, provided herein is a method of transducing heart comprising administering an rAAV having the AAV3B.AR2.08 capsid. In another embodiment, provided herein is a method of transducing heart comprising administering an rAAV having the AAV3B.AR2.10 capsid. In another embodiment, provided herein is a method of transducing heart comprising administering an rAAV having the AAV3B.AR2.11 capsid. In another embodiment, provided herein is a method of transducing heart comprising administering an rAAV having the AAV3B.AR2.12 capsid. In another embodiment, provided herein is a method of transducing heart comprising administering an rAAV having the AAV3B.AR2.13 capsid. In another embodiment, provided herein is a method of transducing heart comprising administering an rAAV having the AAV3B.AR2.14 capsid. In another embodiment, provided herein is a method of transducing heart comprising administering an rAAV having the AAV3B.AR2.15 capsid. In another embodiment, provided herein is a method of transducing heart comprising administering an rAAV having the AAV3B.AR2.16 capsid. In another embodiment, provided herein is a method of transducing heart comprising administering an rAAV having the AAV3B.AR2.17 capsid. In one embodiment, intravenous administration is employed. In another embodiment, ICV administration is employed.

Also provided herein is a method of transducing muscle comprising administering an rAAV having the AAV3B.AR2.01 capsid. In another embodiment, provided herein is a method of transducing muscle comprising administering an rAAV having the AAV3B.AR2.02 capsid. In another embodiment, provided herein is a method of transducing muscle comprising administering an rAAV having the AAV3B.AR2.03 capsid. In another embodiment, provided herein is a method of transducing muscle comprising administering an rAAV having the AAV3B.AR2.04 capsid. In another embodiment, provided herein is a method of transducing muscle comprising administering an rAAV having the AAV3B.AR2.05 capsid. In another embodiment, provided herein is a method of transducing muscle comprising administering an rAAV having the AAV3B.AR2.06 capsid. In another embodiment, provided herein is a method of transducing muscle comprising administering an rAAV having the AAV3B.AR2.07 capsid. In another embodiment, provided herein is a method of transducing muscle comprising administering an rAAV having the AAV3B.AR2.08 capsid. In another embodiment, provided herein is a method of transducing muscle comprising administering an rAAV having the AAV3B.AR2.10 capsid. In another embodiment, provided herein is a method of transducing muscle comprising administering an rAAV having the AAV3B.AR2.11 capsid. In another embodiment, provided herein is a method of transducing muscle comprising administering an rAAV having the AAV3B.AR2.12 capsid. In another embodiment, provided herein is a method of transducing muscle comprising administering an rAAV having the AAV3B.AR2.13 capsid. In another embodiment, provided herein is a method of transducing muscle comprising administering an rAAV having the AAV3B.AR2.14 capsid. In another embodiment, provided herein is a method of transducing muscle comprising administering an rAAV having the AAV3B.AR2.15 capsid. In another embodiment, provided herein is a method of transducing muscle comprising administering an rAAV having the AAV3B.AR2.16 capsid. In another embodiment, provided herein is a method of transducing muscle comprising administering an rAAV having the AAV3B.AR2.17 capsid. In one embodiment, intravenous administration is employed. In another embodiment, ICV administration is employed.

As discussed herein, the vectors comprising the AAV capsids described herein are capable of transducing target tissues at high levels. Thus, provided herein is a method of delivering a transgene to a liver cell. The method includes contacting the cell with an rAAV having a AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 capsid, wherein said rAAV comprises the transgene. In another embodiment, the method includes contacting the cell with an rAAV having any capsid described herein, wherein the rAAV comprises the transgene. In another aspect, the use of an rAAV having the AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 capsid is provided for delivering a transgene to liver.

In another embodiment, a method of transducing CNS tissue is provided. The method includes contacting the cell with an rAAV having an AAV3B.AR2.01, AAV3B.AR2.02, AAV3B.AR2.03, AAV3B.AR2.04, AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17 capsid. In one embodiment, Intra-Cisterna Magna injection is employed.

In one embodiment, the dosage of an rAAV is about 1×10⁹ GC to about 1×10¹⁵ genome copies (GC) per dose (to treat an average subject of 70 kg in body weight), and preferably 1.0×10¹² GC to 2.0×10¹⁵ GC for a human patient. In another embodiment, the dose is less than about 1×10¹⁴ GC/kg body weight of the subject. In specific embodiments, the dose administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg body weight or the subject.

A course of treatment may optionally involve repeat administration of the same rAAV or a different vector (e.g., a rAAV3B.AR2.08 or rAAV3B.AR2.16), particularly for those prenatal, newborn, infant, toddler, preschool, grade-schooler, or teen patients. In one embodiment, those non-adult patients undergo an active proliferating of liver cells, thus requiring repeated administration of an rAAV as described herein which is replication defective. In another embodiment, for a non-adult patient having no native functional hLDLR protein, pre-exposure to a functional hLDLR optionally delivered via another rAAV, particular during the prenatal, newborn or infant stages, may induce a better tolerance and lower immunogenicity to the functional hLDLR, leading to a higher efficacy and efficiency.

In one embodiment, the method further comprises the subject receives an immunosuppressive co-therapy. Such immune suppressant co-therapy may be started prior to delivery of an rAAV or a composition as disclosed, e.g., if undesirably high neutralizing antibody levels to the AAV capsid are detected. In certain embodiments, co-therapy may also be started prior to delivery of the rAAV as a precautionary measure. In certain embodiments, immunosuppressive co-therapy is started following delivery of the rAAV, e.g., if an undesirable immune response is observed following treatment.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include prednisone, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the rAAV administration, or 0, 1, 2, 3, 7, or more days post the rAAV administration. Such therapy may involve a single drug (e.g., prednisone) or co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), two weeks, three weeks, about 60 days, or longer, as needed. In certain embodiments, a tacrolimus-free regimen is selected.

The following examples are illustrative of certain embodiments of the invention and are not a limitation thereon.

### E. EXAMPLES

### Example 1: Materials and Methods

AAV3B variants were isolated by directed evolution (library construction and FRG mouse selection) and further selected/evaluated with barcode evaluation.

### Library Construction

1. A DNA fragment was generated by PCR with Q5 DNA polymerase (NEB) and Primer01+Primer02.
2. Fragments were loaded into an AAV library backbone in a “scarless” way (by using the unique property of restriction enzyme BsmBI whose recognition site is different from its cleavage site) and performed electroporation to generate the initial library L3BSCAR0 (its map and sequence are L3BSC).
3. The plasmid library was transfected into HEK293 cells along with the plasmids pAdAF6 and pRep to produce the packaged AAV library.

| Primer Name | Sequence |
|---|---|
| Primer01 | CGGTCACGTCTCCCAACAGAGCAGTATGGAACTGTCGCARMCV |
| | ACMWCCAGVRCMRCRRCRMAGCTCCCAHCABASRGACAGTCA |
| | ATGATCAGGGAGCC (SEQ ID NO: 35) |
| Primer02 | GCCAGTCGTCTCCGGTCTTGCCACACCATGCCAGGTAAGGCTCC |
| | CTGATCATTGACTGT (SEQ ID NO: 36) |
| Primer03 | CGGTCACGTCTCCCAACAGAGCAGTATGGAACTGTCGCA (SEQ |
| | ID NO: 37) |
| Primer04 | GCCAGTCGTCTCCGGTCTTGCCACACCATGCCAGGTAA (SEQ ID |
| | NO: 38) |
| Primer05 | GGCGAACAGCGGACACCGATATGAA (SEQ ID NO: 39) |
| Primer06 | GGCTCTCGTCGCGTGAGAATGAGAA (SEQ ID NO: 40) |

### FRG Mouse Selection

To select the library for human liver tropism, the packaged AAV library was injected intravenously into human-hepatocytes-xenografted Fah^{-/-}/Rag2^{-/-}/Il2rg^{-/-} (FRG) mice (Yecuris, Oreg., USA) at a dose of ≥1×10¹² GC/mouse. Four weeks later, hepatocytes were harvested the with collagenase perfusion. Human hepatocytes were then enriched by treating the hepatocytes with anti H-2kb antibody coated magnetic beads to remove the murine hepatocytes. The AAV signal was then retrieved from the human hepatocytes by RT-PCR with Q5 DNA polymerase, Primer03 and Primer04 and then loaded into the library backbone to generate a new library (map and sequence still L3BSC) for the next round of selection.

The diversity change (variant frequency changes) was monitored by next generation sequencing (NGS).

After two rounds of FRG mouse selection, we picked sixteen variants (the variants have the highest frequencies) and used the barcode evaluation system to evaluate their performance.

### The Barcode Evaluation System

DNA barcodes can be used to evaluate multiple testing articles, each tagged with a DNA barcode, at the same time, by reading the frequency changes of each barcode before and after the treatments. Our barcode evaluation system was used to evaluate the performance of various AAV capsids at the same time. The key component of the system is a series of barcoded cis plasmids, each plasmid carrying a unique 6-bp DNA barcode. The cis plasmids were identical except the DNA barcodes. The backbone of the cis plasmids was the cis plasmid for self-complementary AAV vectors—a transgene cassette flanked by a defective ITR (ΔITR) at the cassette's 5′ end and a normal ITR at its 3′ end. The transgene cassette was CB8 promoter—SV40 intron—eGFP—SV40 polyA signal. Further, all of the ATG codons within the eGFP transgene were removed so that no protein was expressed (the resulting ORF is named as dEGFP—dead eGFP) and a 6-bp barcode was inserted right after the dEGFP.

A barcoded cis plasmid was mixed with pAdAF6 and the trans plasmid carrying an AAV capsid gene to be tested for triple-transfection into HEK293 cells to produce an AAV vector prep. Each vector in the prep had the tested capsid as its capsid and carries in its genome the DNA barcode from the cis plasmid. Therefore, the barcode was linked to the tested capsid.

For multiple capsids to be tested, the AAV vector preps were produced individually so each capsid linked to a unique DNA barcode. The preps were then pooled together for animal studies. After the pooled vectors were injected into animals, various tissues were then collected and preserved in RNAlater solution. PCR and RT-PCR can then be carried out and the barcode frequencies are then read by NGS.

| Barcode Name | Barcode sequence |
|---|---|
| BC01 | ATCACG |
| BC02 | CGATGT |
| BC03 | TTAGGC |
| BC04 | TGACCA |
| BC05 | ACAGTG |
| BC06 | GCCAAT |
| BC07 | CAGATC |
| BC08 | ACTTGA |
| BC09 | GATCAG |
| BC10 | TAGCTT |
| BC11 | GGCTAC |
| BC12 | CTTGTA |

### Overview

1. Collect tissues into RNAlater (Qiagen). Store the preserved samples at −20° C. or −80° C.
2. Use Trizol (Ambion) to extract RNA, by following the manufacturer's instructions.
3. DNase I treatment: 100 μL reaction system, 2 μL of DNase I recombinant, RNase-free (Roche, 10 U/μL), ≤100 μg Trizol-extracted RNA, 37° C. 1 hour.
4. Use RNeasy Mini Kit (Qiagen) to do the cleanup, by following the manufacturer's instructions.
5. Follow RT's manual (High Capacity cDNA Reverse Transcription Kit, Applied Biosystems) to do the RT, with oligo dT (Invitrogen, Cat #18418012, 0.1 μg oligo dT/1 μg total RNA). 1 μg total RNA/10 μL reaction. RT—controls included.
6. PCR: Q5 DNA polymerase. For 50 μL reaction, ≤5 μL cDNA, 2.5 μL of 10 μM Primer05 and 2.5 μL of 10 μM Primer06. 98° C. 30 s, x cycles of (98° C. 10 s, 72° C. 17 s), 72 120 s, 4° C. infinite.
7. The PCR products are read by NGS to obtain the frequencies of the barcodes in the samples.

### Example 2: Development of AAV3B Variants with Improved Liver Transduction in Nonhuman Primates by Directed Evolution

### Overview

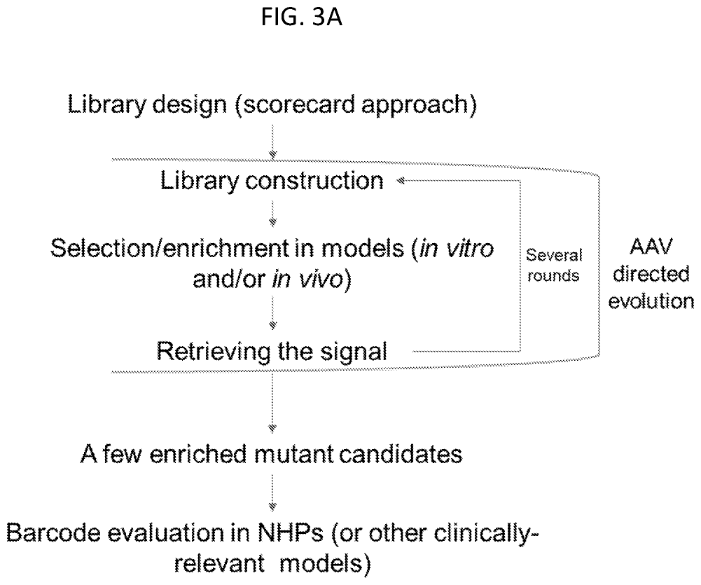

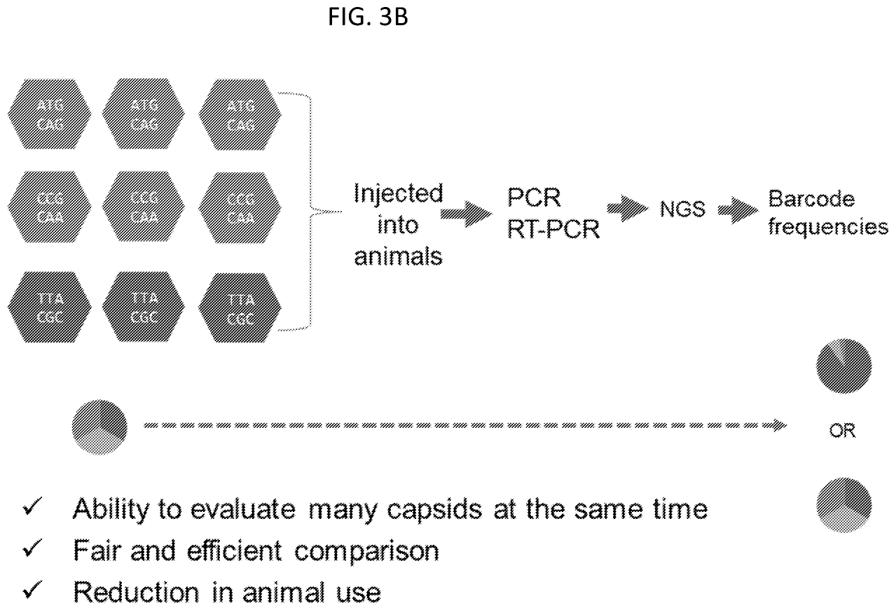

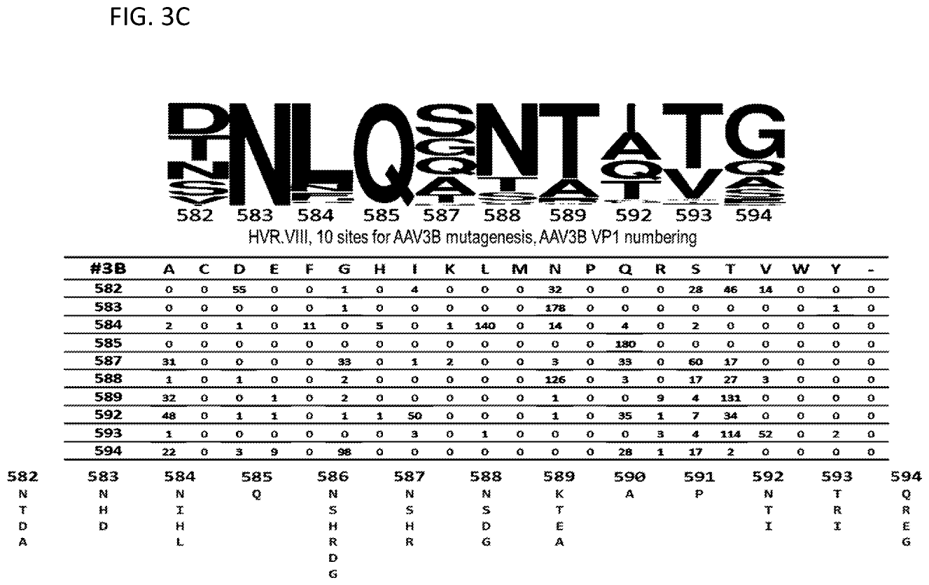

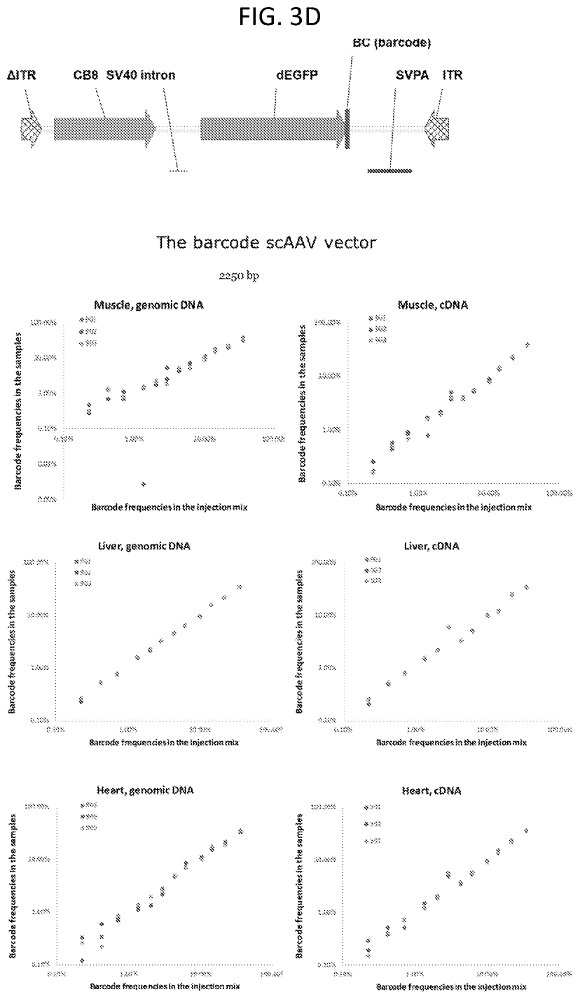

A scorecard approach was used to evaluate diversity in the AAV3B hyper variable region (HVR) VIII (FIG. 3A-FIG. 3C). We then conducted selections in human-hepatocytes-xenografted Fah^{-/-}/Rag2^{-/-}/Il2rg^{-/-} (FRG) mice, by injecting the libraries intravenously and retrieving AAV cDNA from human hepatocytes isolated from those mice to prepare new libraries for the next rounds. Sixteen AAV3B variants that showed increases in relative frequencies were evaluated in nonhuman primates (NHPs) with a validated barcodes system. Most of the sixteen variants were better than AAV3B in terms of liver transduction, with some showing high liver specificity. Two variants were further evaluated with a therapeutic transgene for liver gene therapy in NHPs and the preliminary results confirmed the NHP barcode evaluation results.

Creation of Vectors with High Tropism to Human Hepatocytes and Low Recognition by NAbs Based on Engineered Forms of AAV3B.

We examined the HVR VIII of AAV3B and focused on the non-conserved amino acids compared to the other AAV serotypes. The AAV3B VP1 was aligned with that of 180 other AAVs, and 10 amino acids between 582-594 were chosen based on their variability among the aligned sequences. In order to maximize the viability of the mutant, degenerate codons were designed with the intention to introduce alternative amino acids appeared in other AAVs at the aligned position. The mutant capsid sequences were cloned into the AAV capsid expression plasmid, mixed with helper plasmid (pAdAF6) and pRep, and then transfected into 293 cells to produce the packaged AAV library.

FRG mice with human hepatocyte xenografts were used to select AAV mutants with human liver tropism from the library. FRG stands for triple mutant of Fah(−/−), Rag-2(−/−) and IL2rg(−/−). The Fah is a gene in the catabolic pathway for tyrosine, and its deletion leads to liver damage unless the drug 2-(2-nitro-4-trifluoromethylbenzoyl) 1,3-cyclohexedione (NTBC) is supplemented to block the accumulation of the toxic metabolite. When NTBC is withdraw, hepatocyte from human donor can be introduced, and the double knockout of Rag-2(−/−) and IL2rg(−/−) cause severe immune-deficiency and allows the survival of the human hepatocyte. FRG mice with repopulated human hepatocyte were purchased from Yecuris (Tigard, Oreg., USA) and injected with the library intravenously at, minimally, 1×10¹² GC per animal. At day 28, the livers were perfused with collagenase to harvest the hepatocytes. Among the 4 animals injected, up to 40 million human hepatocytes were recovered with over 95% viability. Magnetic beads with anti-H2-kb, which is a mouse specific marker, were used to remove mouse hepatocytes from the harvested cells. Primers targeting the designed mutations were used to amplify DNA fragments containing HVR VIII via RT-PCR. The DNA fragments were cloned back into the capsid expression plasmid to proceed with the next round of screening.

To select vectors with high tropism to human hepatocytes from the library, we injected an AAV3B library into FRG mice xenografted with human hepatocytes. RNA fragments recovered from the isolated human hepatocytes was subjected to RT-PCR using primers flanking the engineered HVRVIII region and re-cloned into a cis-plasmid designed to express AAV3B VP1 for repeat selection. We performed next generation sequencing (NGS) on the libraries from before selection (denoted as AR0), after first round (AR1) and the plasmid after second round of humanized FRG selection (AR2) and examined the frequency of each variant (FIG. 3D). The eighteen AAV3B variants with the highest frequencies in AR2 were found to have consistently increasing normalized frequencies in the library sequenced, which suggests that these variants may have relative advantages in transducing human hepatocytes. The amino acid and DNA sequences are provided in the sequence listing.

### Example 3: AAV3B Variants—Barcode Evaluation

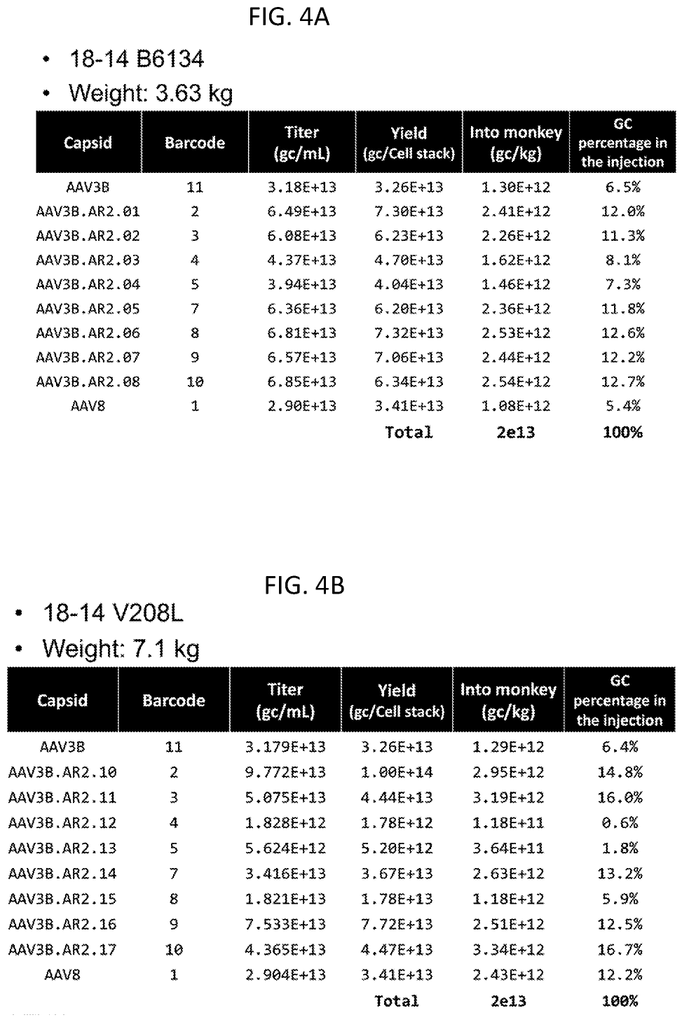

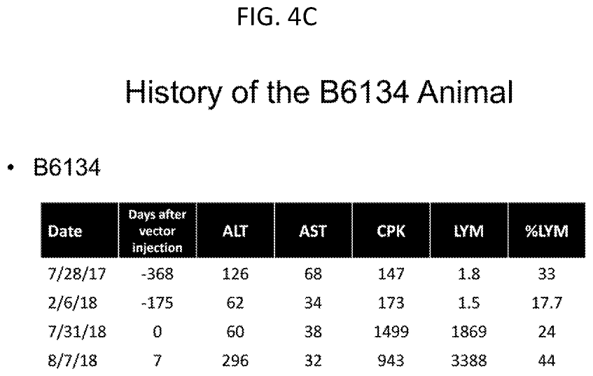

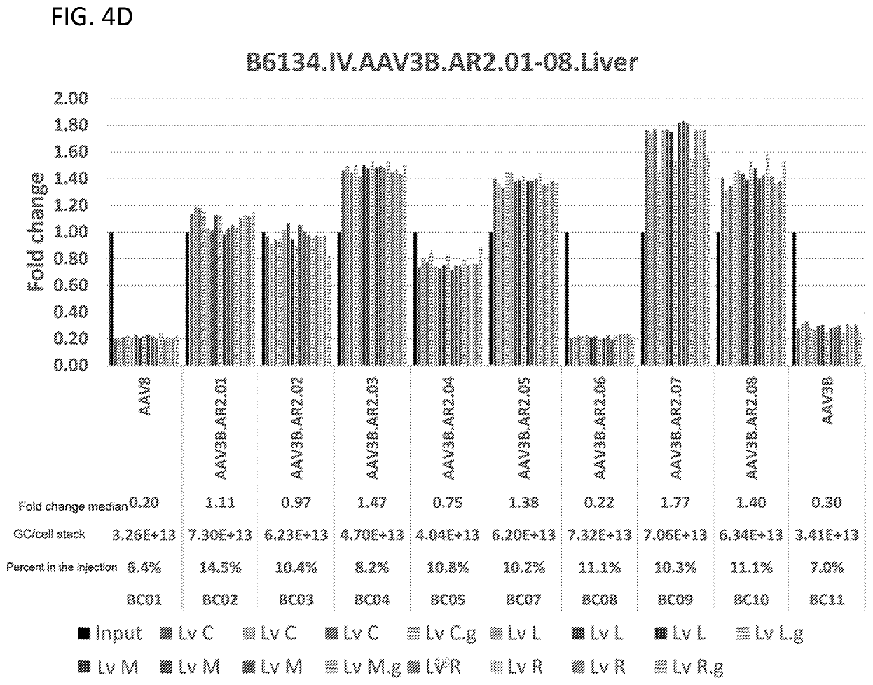

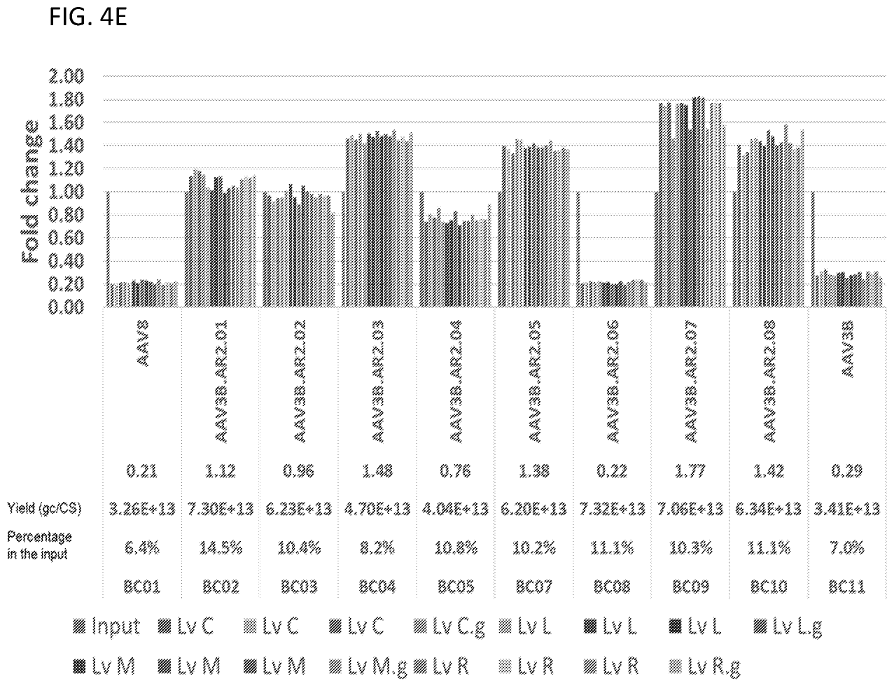

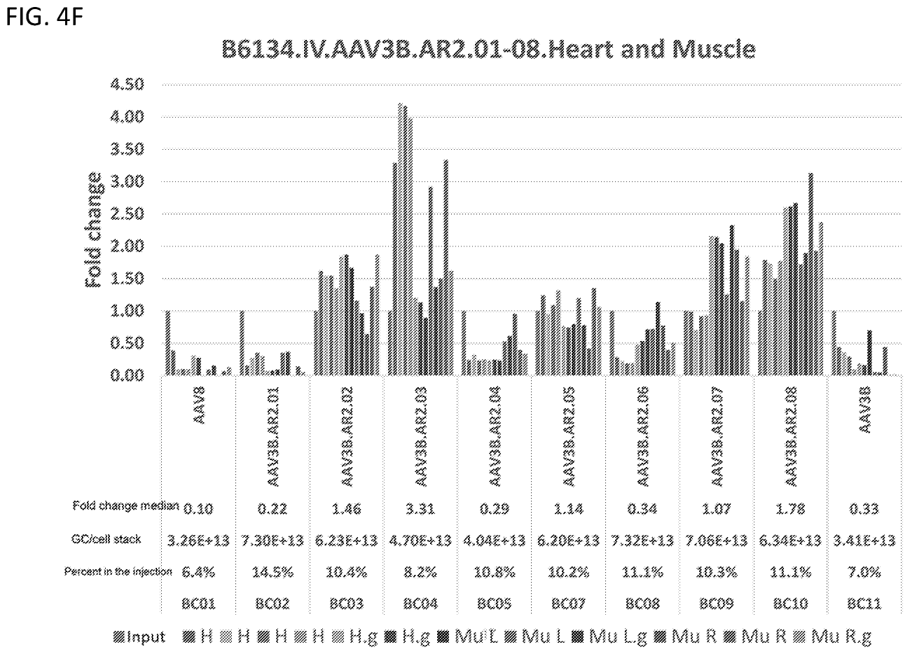

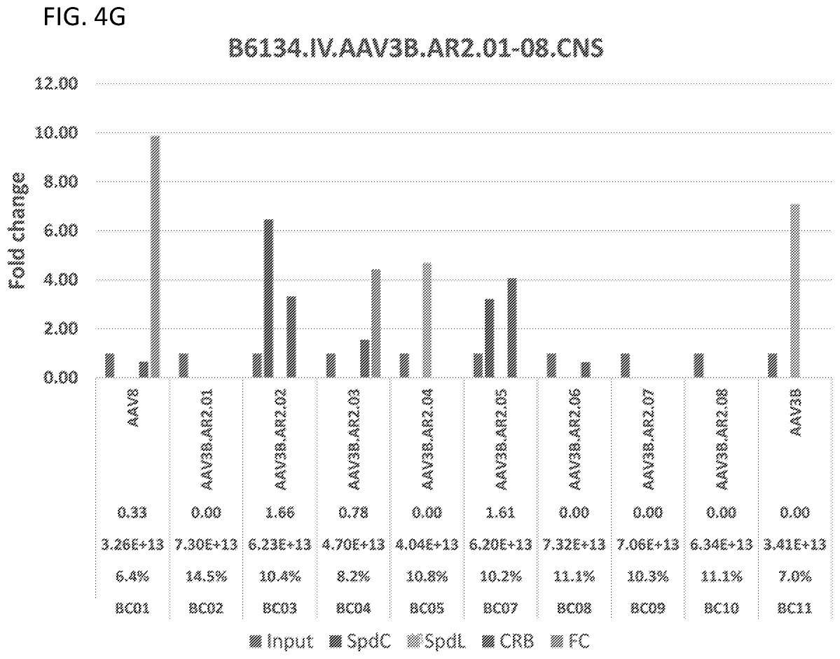

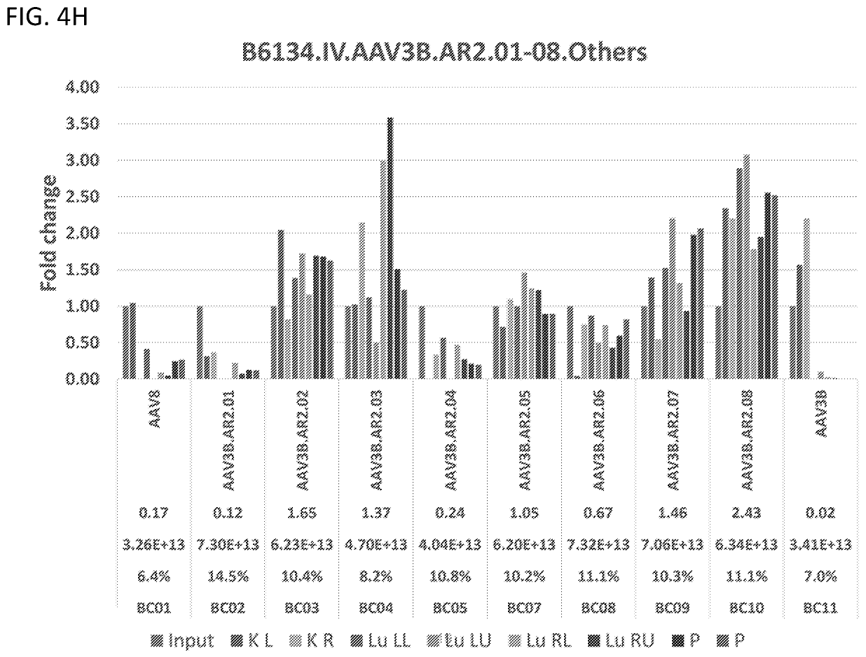

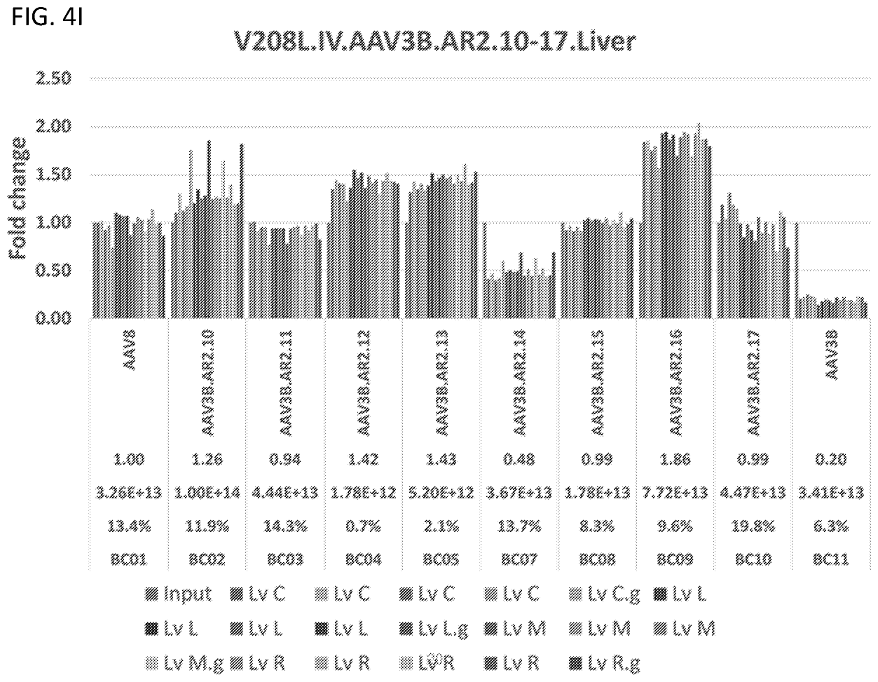

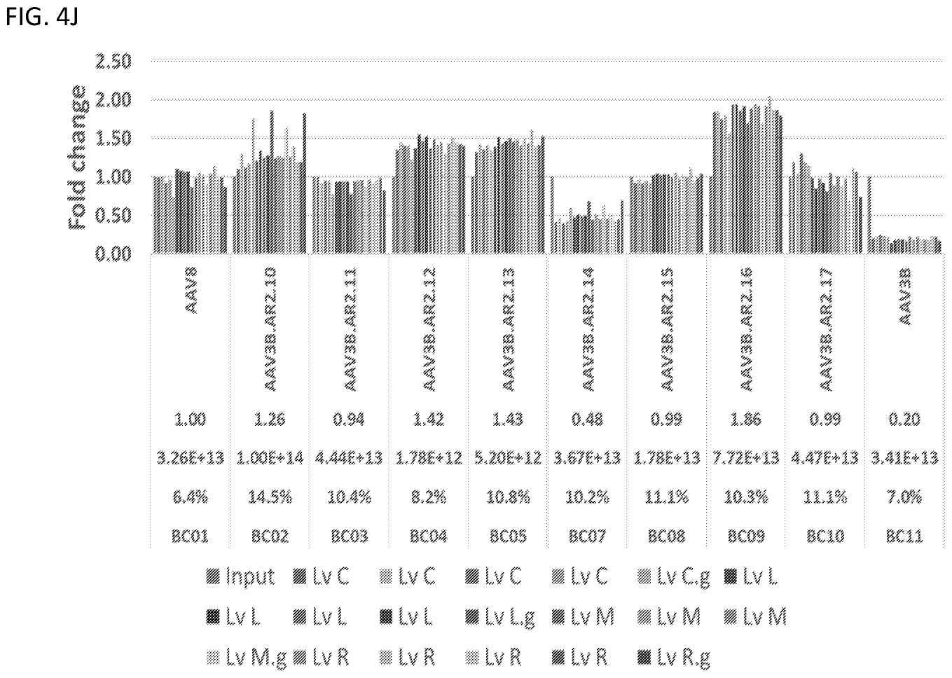

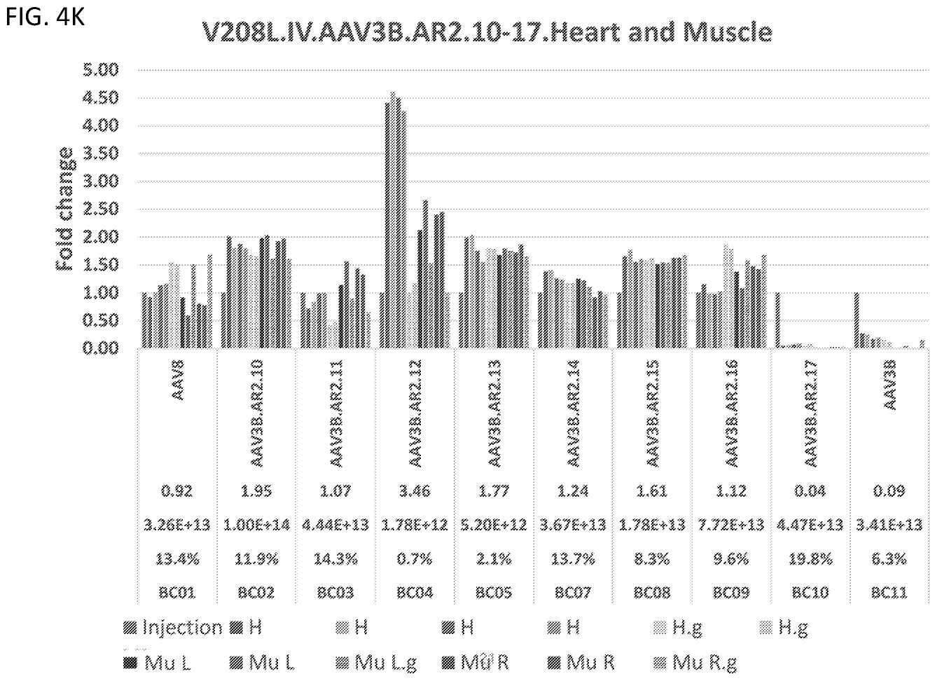

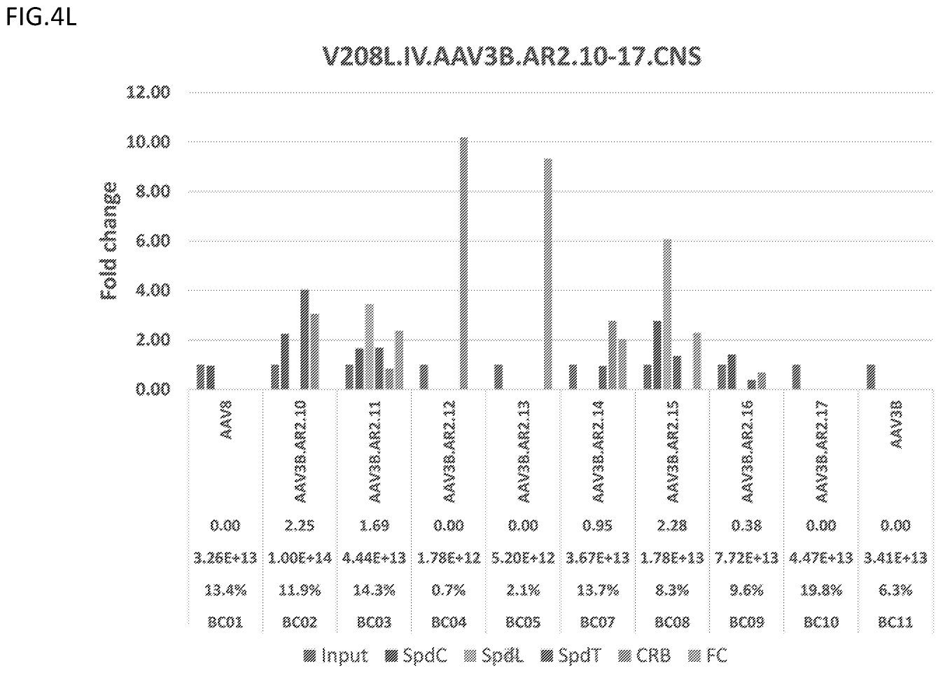

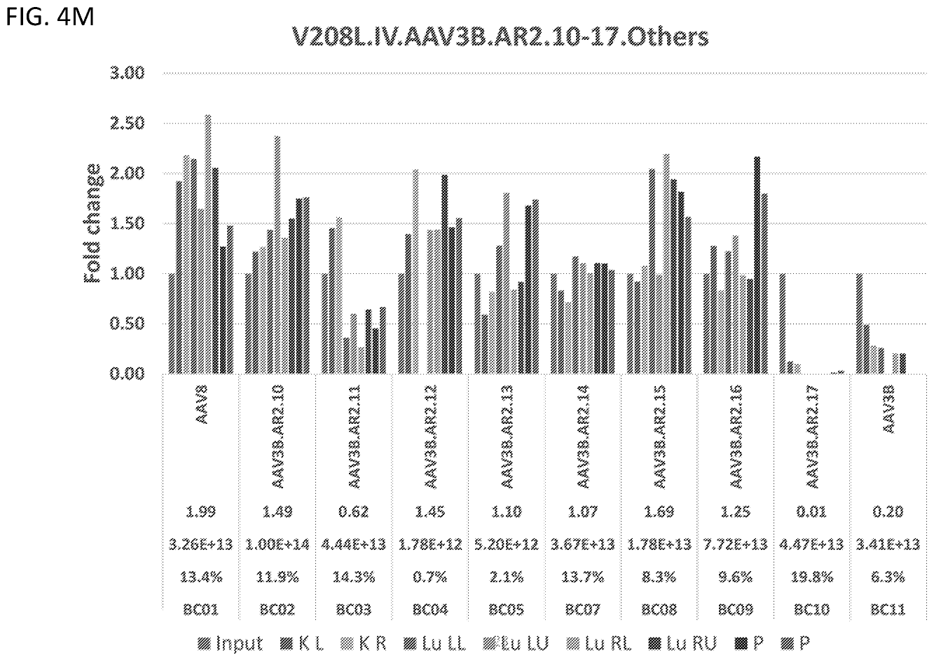

Barcoded AAV3B variants were injected into two NHP (B6134 and V208L) at a dosage of 2×10¹³ gc/kg IV. Details relating to injected vectors are shown in FIG. 4A and FIG. 4B. Seven days post vector administration, tissues were harvested. Barcode fold changes were compared. For animal B6134, fold changes for each variant tested are shown in FIG. 4D and FIG. 4E (liver), FIG. 4F (heart and muscle), FIG. 4G (CNS), and FIG. 4H (other tissues). For animal V208L, fold changes for each variant tested are shown in FIG. 4I and FIG. 4J (liver), FIG. 4K (heart and muscle), FIG. 4L (CNS), FIG. 4M (other tissues).

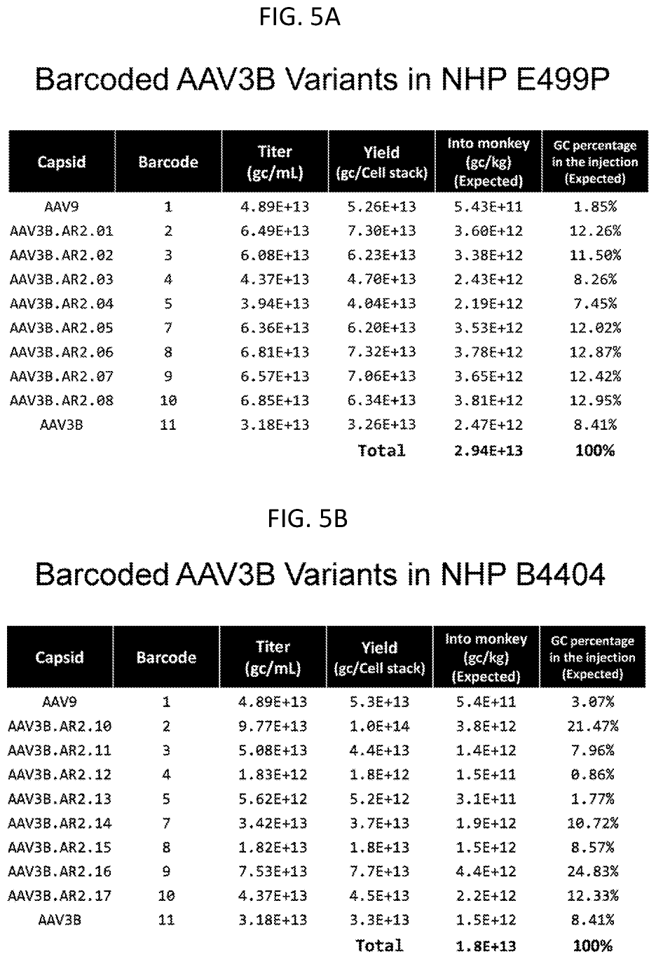

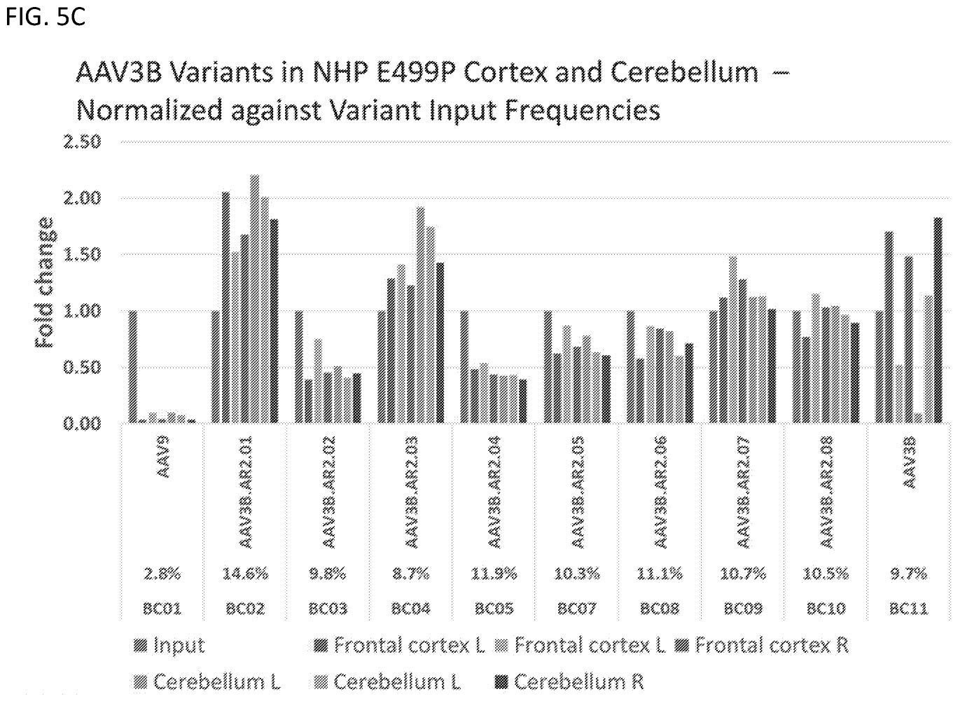

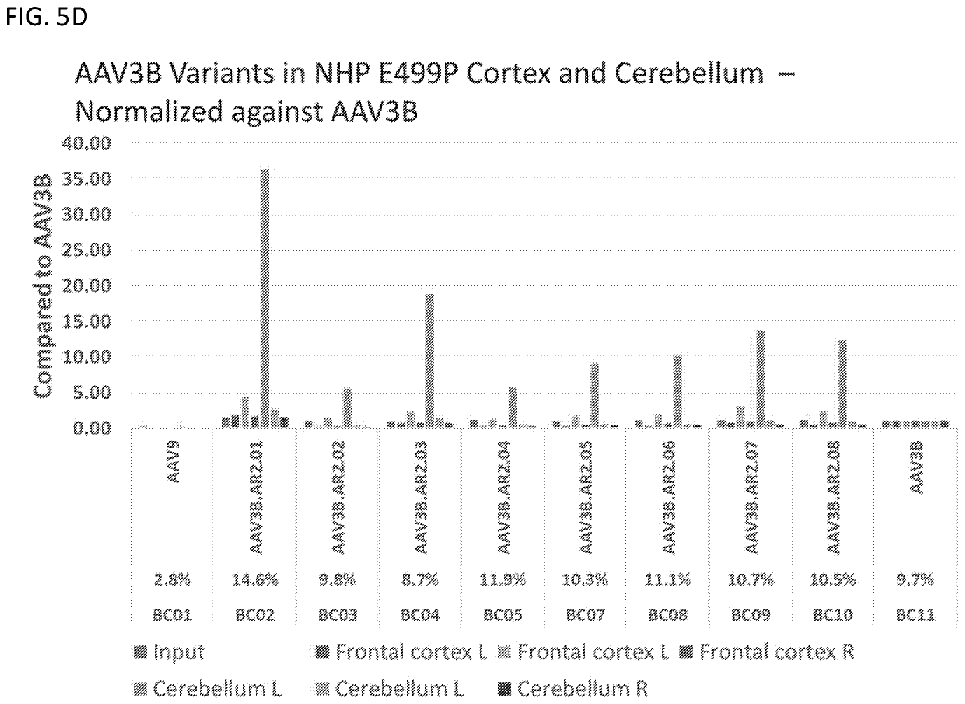

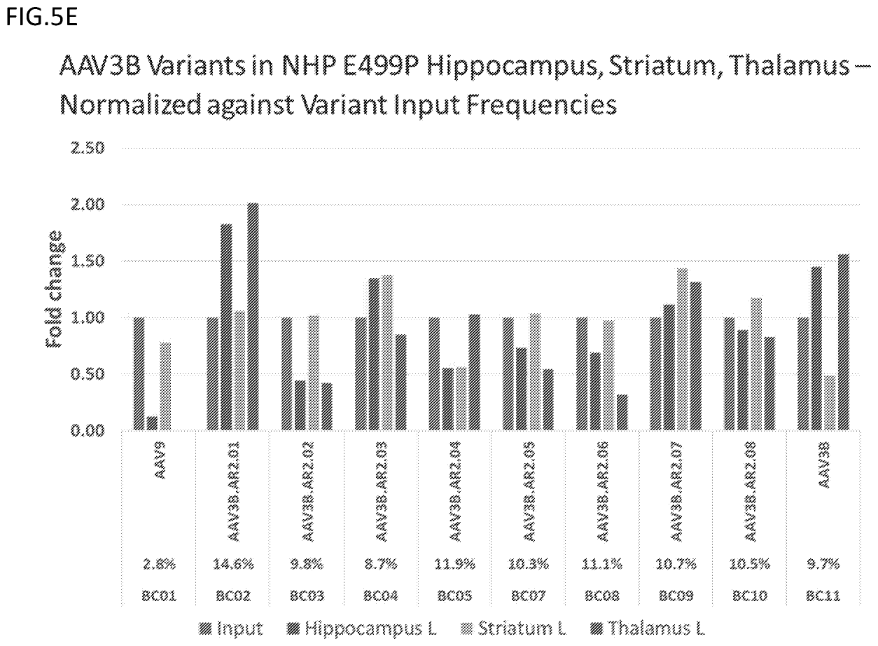

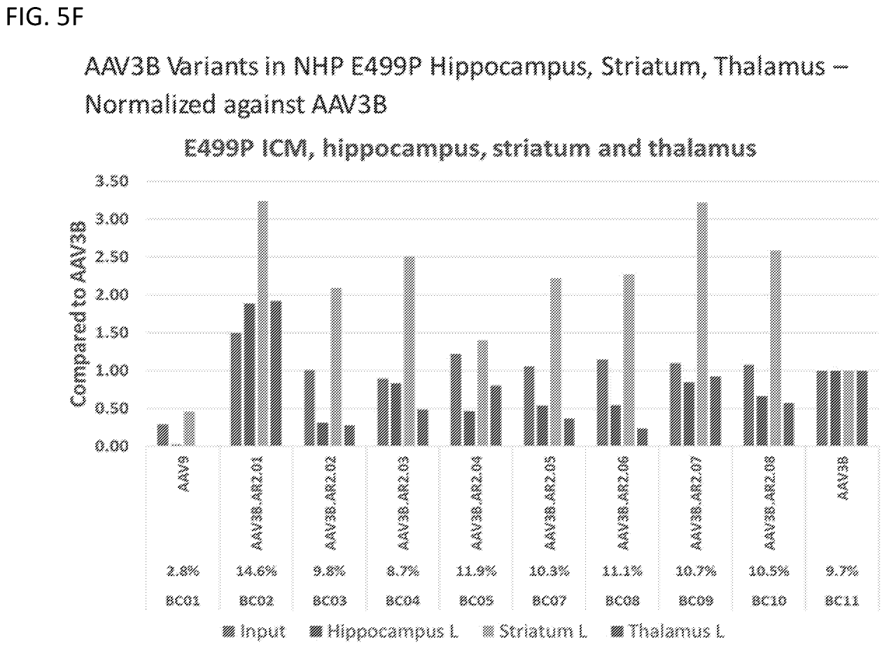

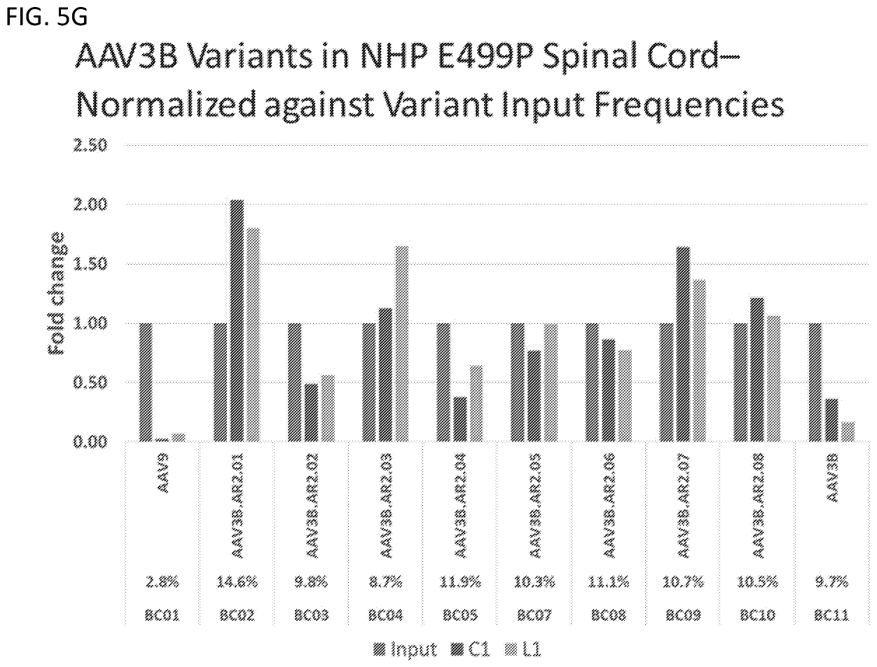

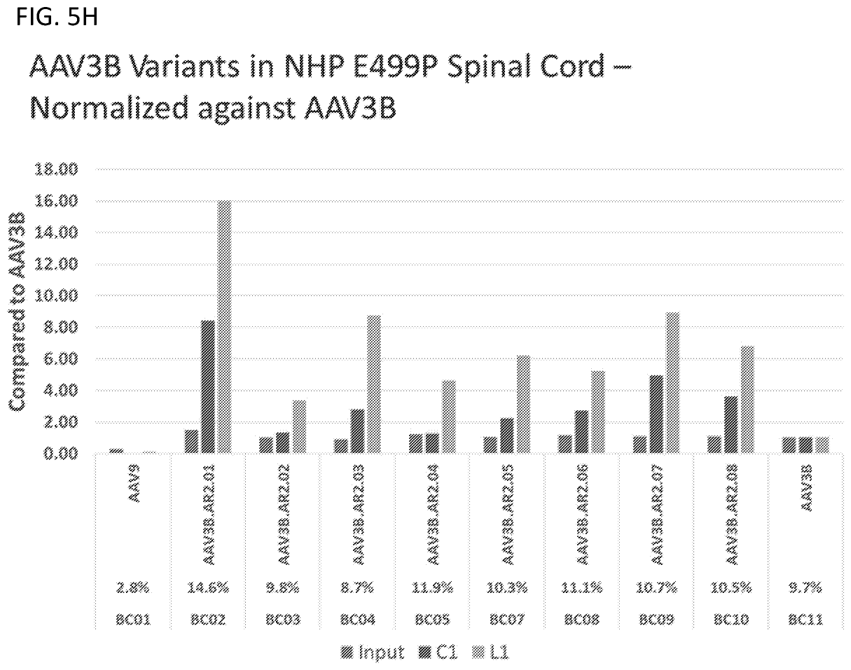

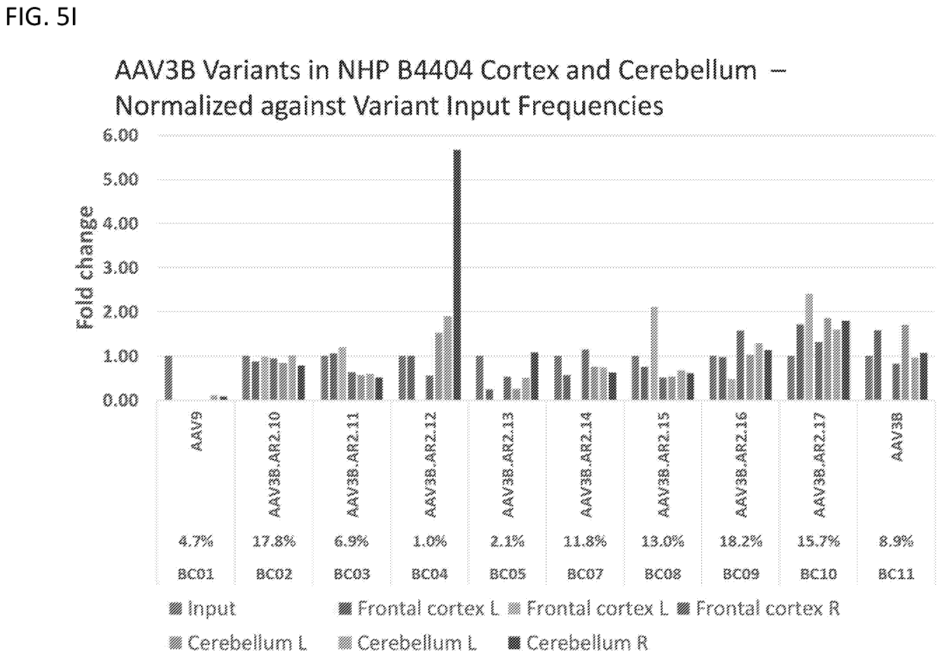

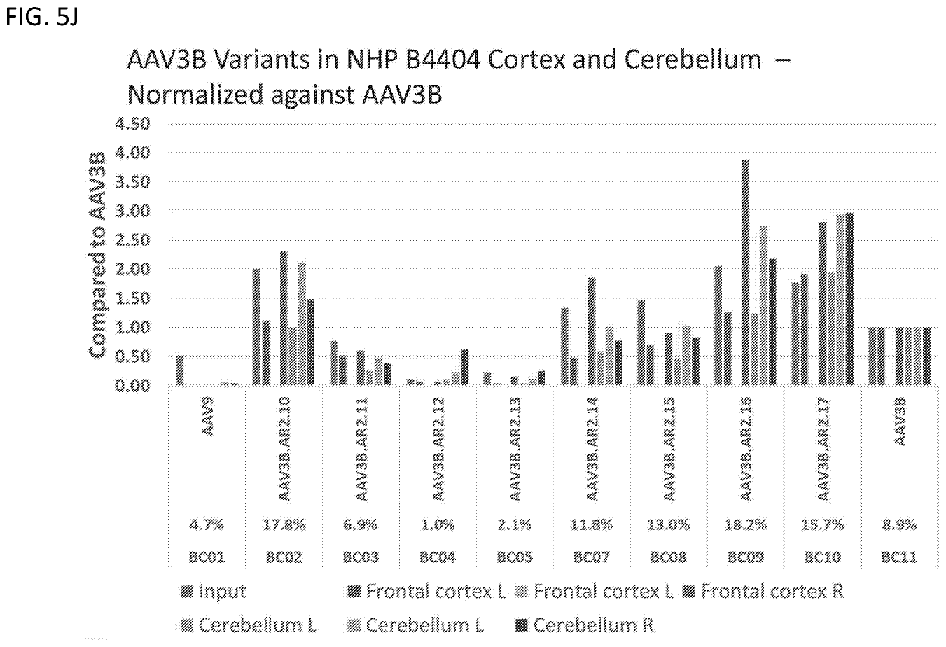

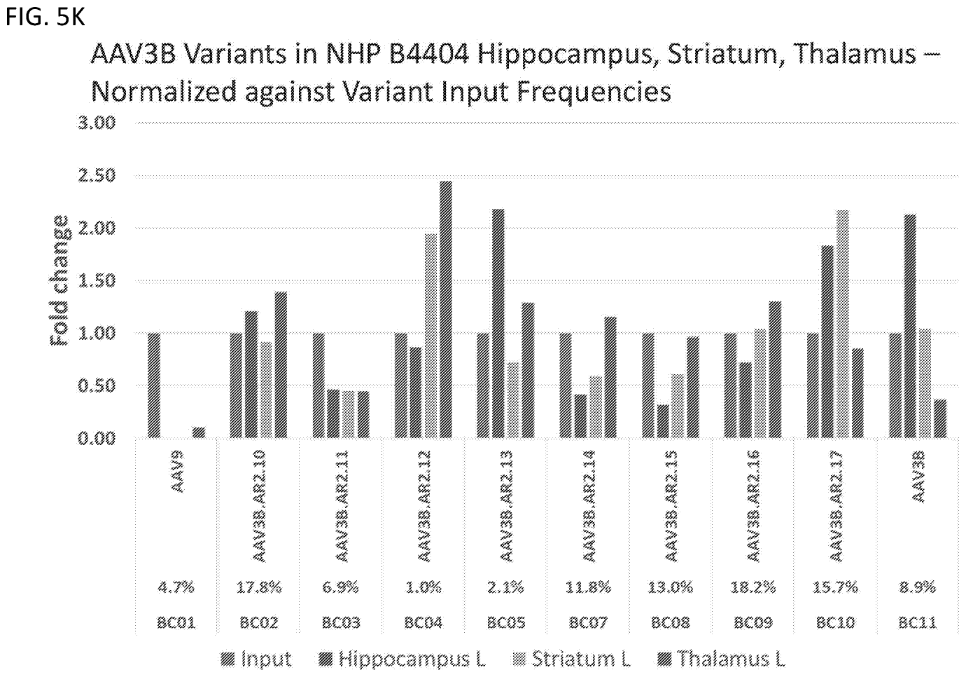

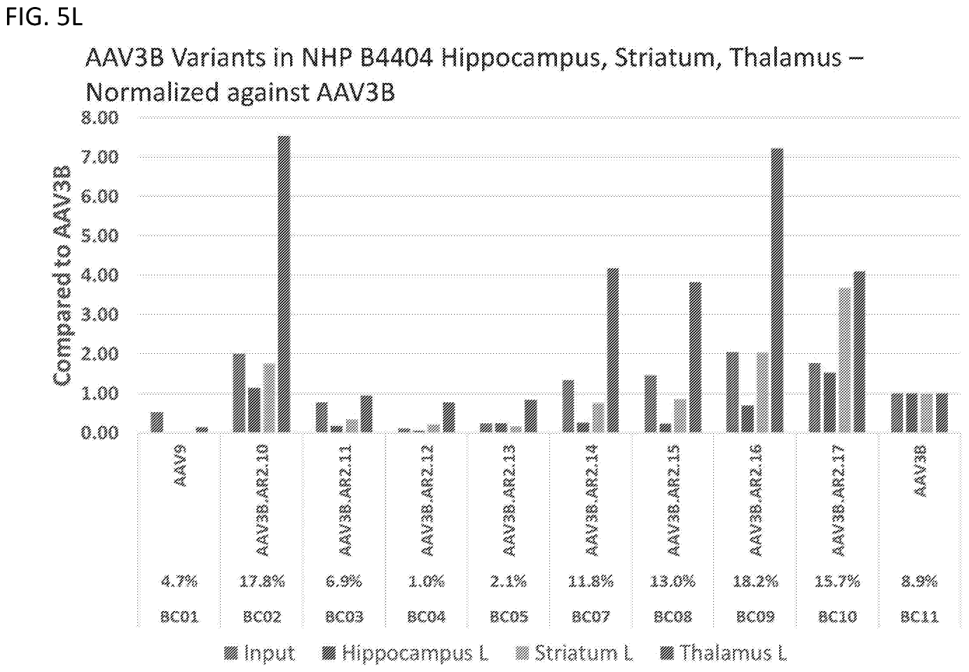

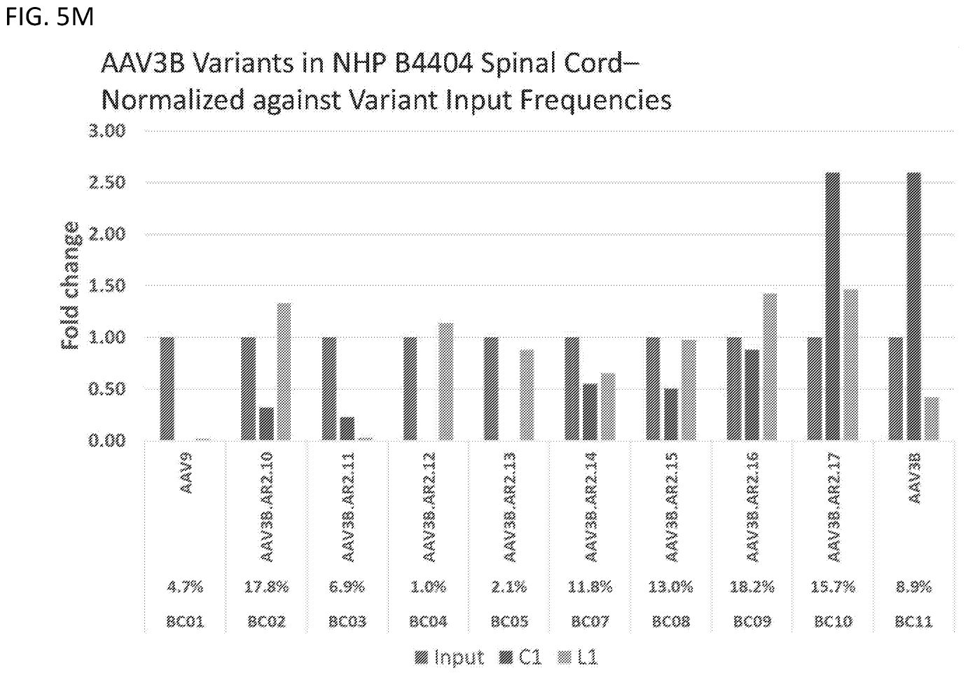

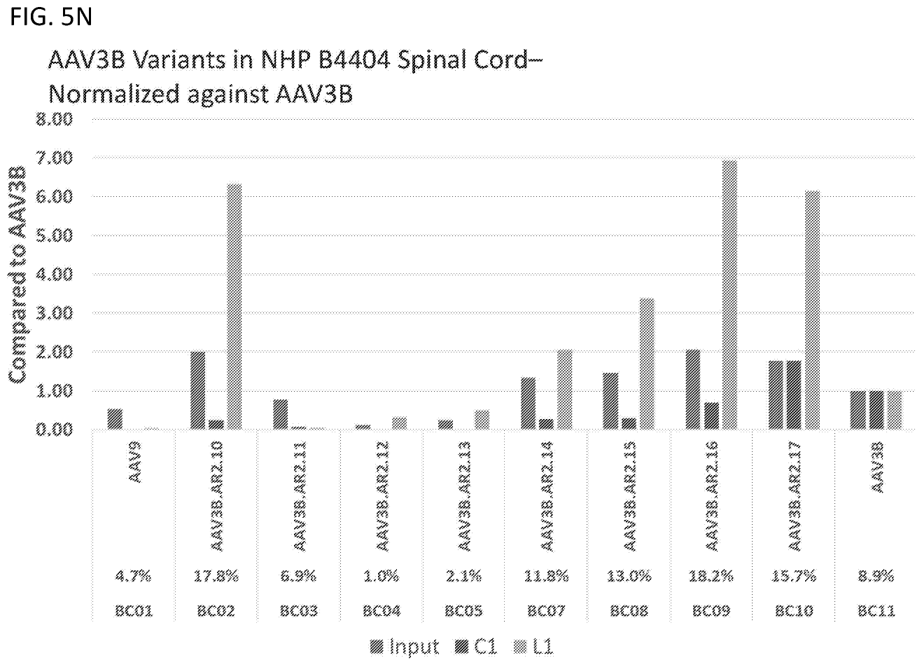

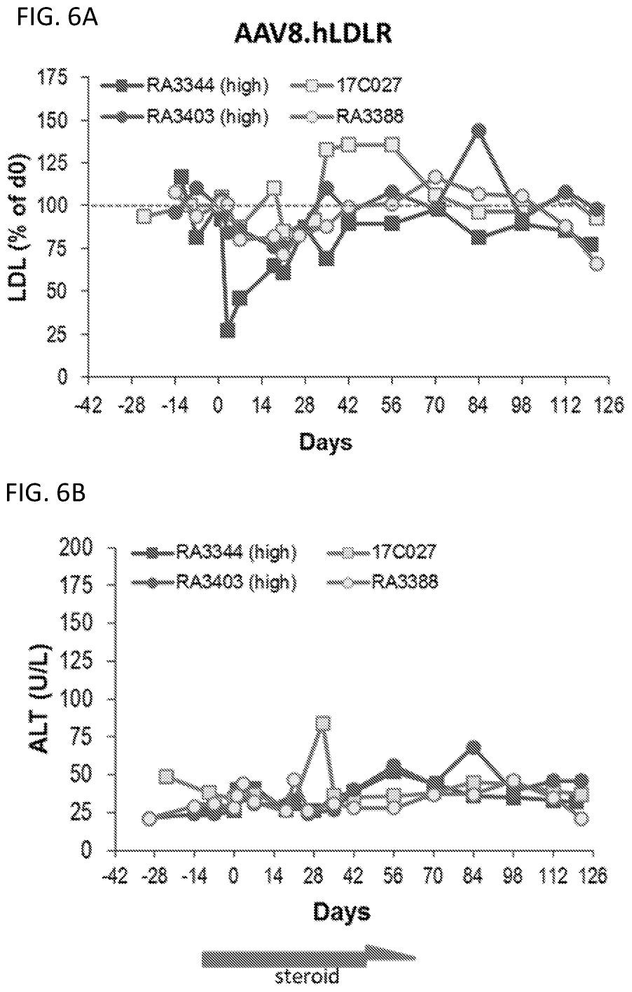

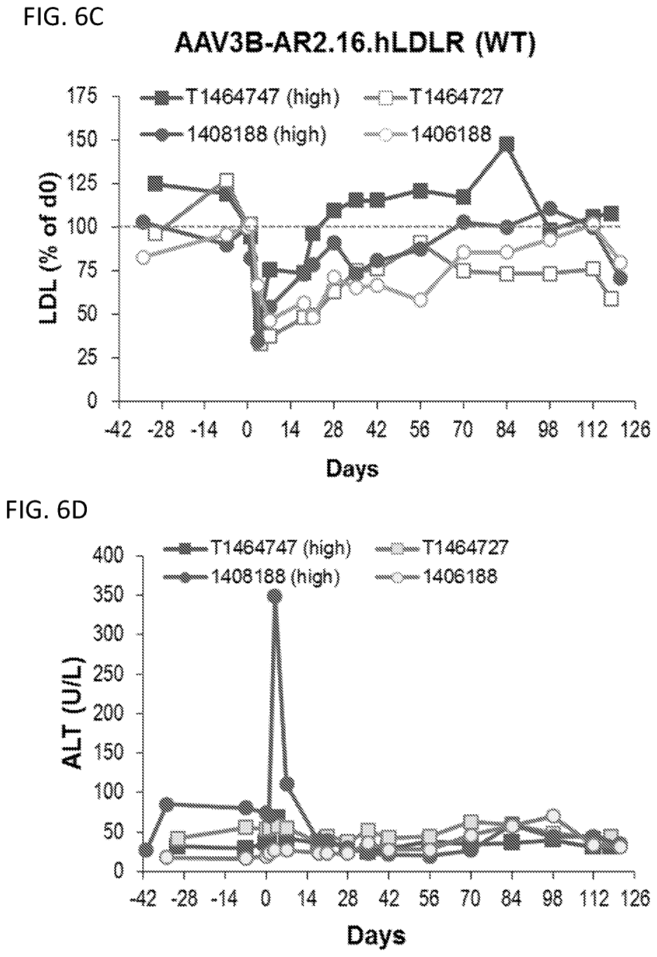

The barcoded AAV3B variants were also injected into two NHP (E499P and B4404) at a dosage of ˜1.8×10¹³ and ˜2.9×10¹³ gc/animal via intra-cisterna magna (ICM) injection (FIG. 6A). Details relating to injected vectors are shown in FIG. 5A and FIG. 5B. Fourteen days post vector administration, tissues were harvested. Barcode fold changes were compared.

Fold changes in cortex and cerebellum are shown in FIG. 5C (normalized against variant input frequencies) and FIG. 5D (normalized against AAV3B) for animal E499P. Fold changes in hippocampus, striatum, thalamus are shown in FIG. 5E (normalized against variant input frequencies) and FIG. 5F (normalized against AAV3B) for animal E499P. Fold changes in spinal cord are shown in FIG. 5G (normalized against variant input frequencies) and FIG. 5H (normalized against AAV3B) for animal E499P.

Fold changes in cortex and cerebellum are shown in FIG. 5I (normalized against variant input frequencies) and FIG. 5J (normalized against AAV3B) for animal B4404. Fold changes in hippocampus, striatum, and thalamus are shown in FIG. 5K (normalized against variant input frequencies) and FIG. 5L (normalized against AAV3B) for animal B4404. Fold changes in spinal cord are shown in FIG. 5M (normalized against variant input frequencies) and FIG. 5N (normalized against AAV3B) for animal B4404.

### Example 4: Characterization of a AAV3B Variants for Treatment of Hypercholesterolemia

We selected AAV3B.AR2.08 and AAV3B.AR2.16 to further evaluate their potential as second-generation clinical candidates using the codon-optimized, triple mutation hLDLR. The following vectors were generated:
a. AAV8.TBG.PI.hLDLR.rBG
b. AAV3B.AR2.16.TBG.PI.hLDLR.rBG
c. AAV3B.AR2.16.TBG.IVS2.hLDLR011.bGH
d. AAV3B.AR2.16.TBG.IVS2.hLDLR011-triple.bGH
e. AAV3B.AR2.08.TBG.IVS2.hLDLR011-triple.bGH
f. AAV3B.TBG.IVS2.hLDLR011-triple.bGH

The rAAV are named after its capsid and vector genome in a format of “capsid.vector genome”. An AAV capsid may be an “AAV8”, “AAV3B.AR2.08” or “AAV3B.AR2.16” capsid. The vector genomes are further noted based on their promoter, intron, hLDLR coding sequence and polyA sequence separated by “.”.

As used herein, “TBG” indicates a TBG promoter. “PI” refers to a chimeric intron with Genbank #U47121 (Promega Corporation, Madison, Wis.), while “IVS2” means a human β-globin intron 2. The term “rBG” provides a rabbit beta-globin polyadenylation signal in the rAAV while bGH stands for a polyadenylation signal from the bovine growth hormone.

With respect to the LDLR coding sequence, “hLDLR” or “LDLR” indicates that the coding sequence is the human wild-type coding sequence encoding a wild-type hLDLR protein; “hLDLR011” or “LDLR011” indicates the engineered coding sequence encoding a wild-type hLDLR protein; and “hLDLR011-triple” or “hLDLR011.triple” or “LDLR011.trip” means the engineered coding sequence encoding a hLDLR protein with three amino acid substitutions, i.e., L318D/K809R/C818A.

When referring to a vector genome or an rAAV particle without specifying a capsid, a similar format is used as the following: “AAV.promoter(optional).intron(optional).hLDLR coding sequence.polyA (optional)”.

### A. Non-Human Primate (NHPs) Study

Three rAAV were tested, including AAV8.TBG.PI.hLDLr.rBG.KanR, or AAV3B-AR2.08.TBG.IVS2.hLDLR011 (L318D, K809R,C818A).bGH, or AAV3B-AR2.16.TBG.IVS2.hLDLR011 (L318D, K809R,C818A).bGH. Each of the rAAV was delivered IV to four animals. Two animals received 2.5×10¹³ GC/kg (noted as “high” in the drawings) and two animals received 7.5×10¹² GC/kg (which is referred to as the “low dose” or “lower dose”).

Starting on the day of rAAV administration (day 0), animals received Prednisolone (1 mg/kg/day) orally for transient immune suppression. At approximately eight weeks post vector administration, animals were tapered off Prednisolone by gradual reduction of daily dose. The LDL and PCSK9 levels of injected animals were measured to evaluate the efficacy of the vectors.

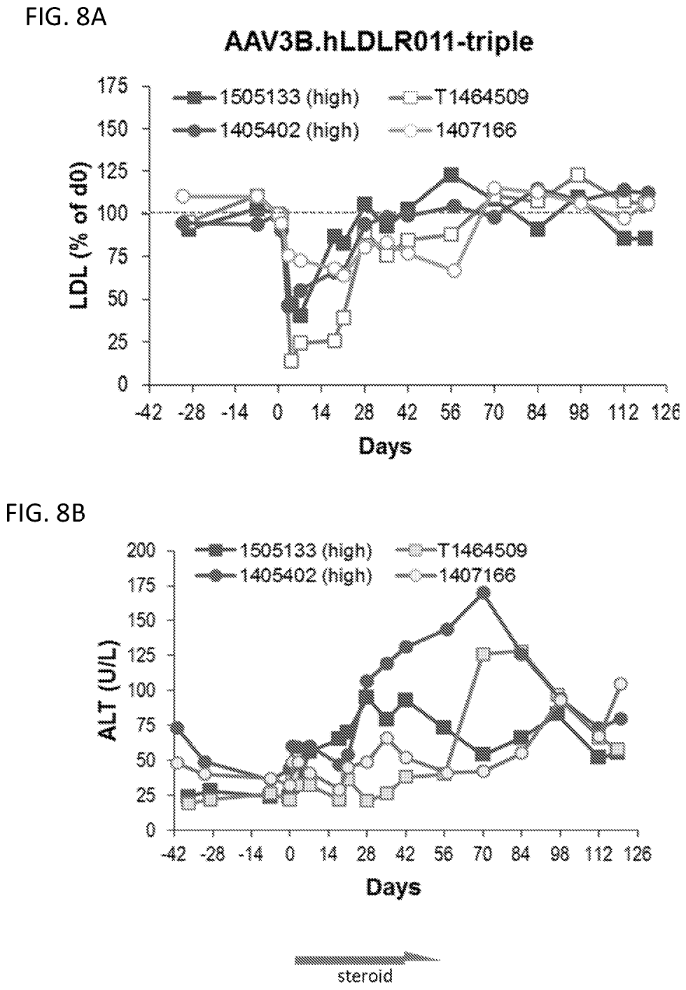

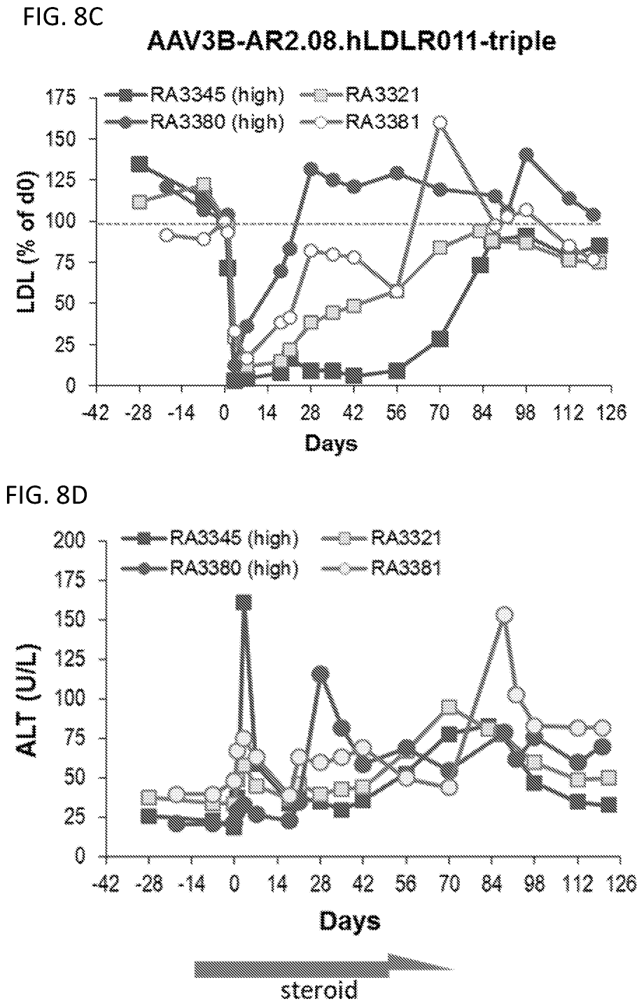

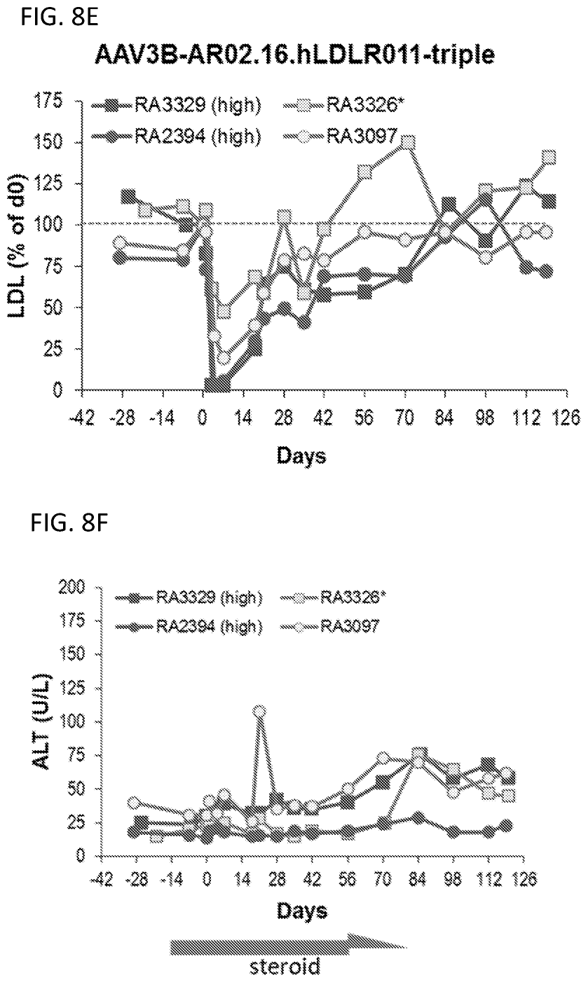

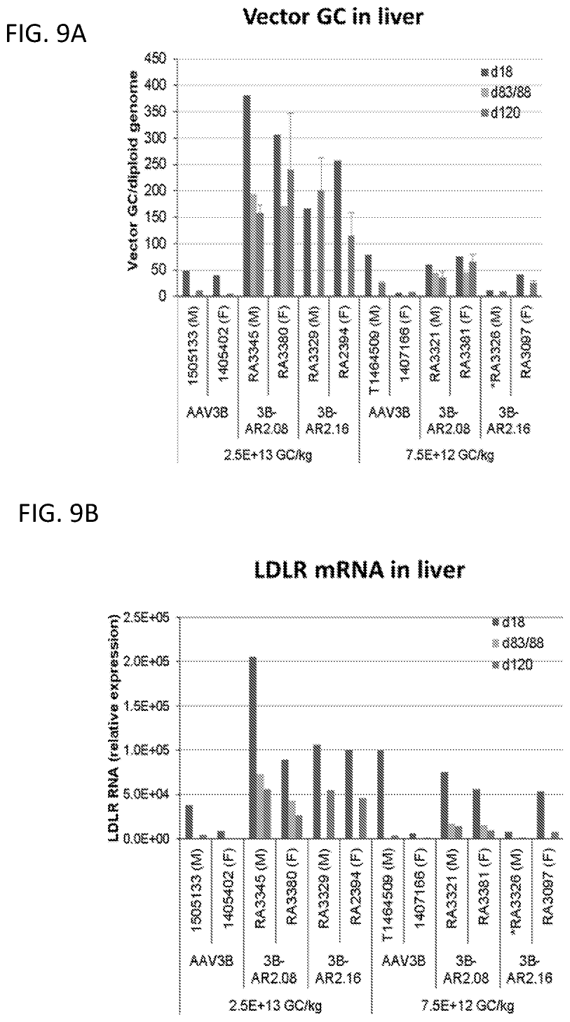

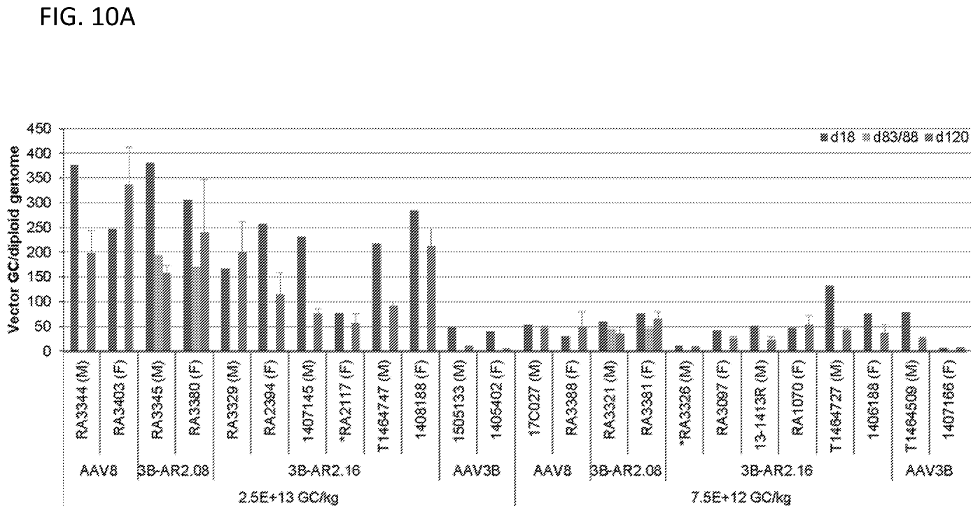

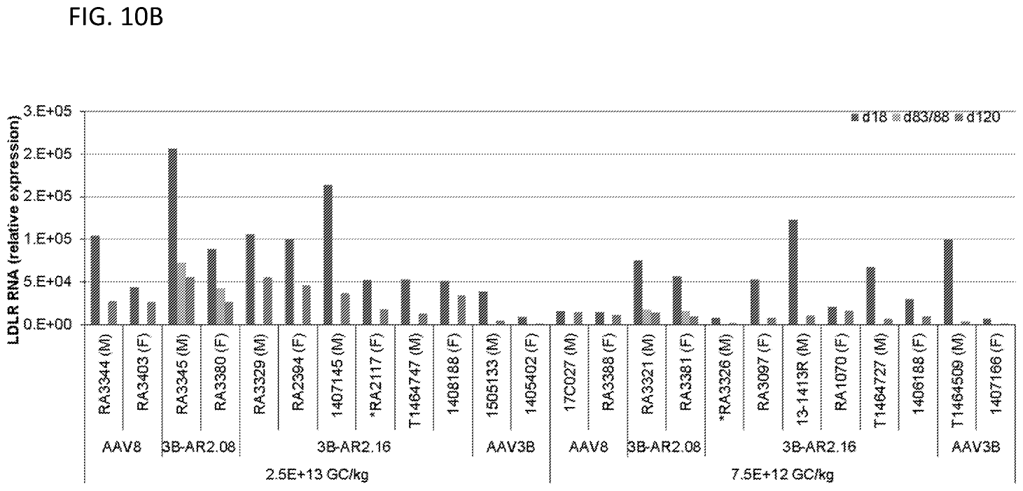

Each animal received at least one liver biopsy on day 18 for the purpose of monitoring the stability of the transgene. The vector genome copies in biopsy samples showed dose dependency. The lower dose of AAV3B-AR2.08 resulted in higher vector genome copies than AAV8 or AAV3B-AR2.16. Two animals in the AAV3B-AR2.16 group (marked with asterisk in FIG. 8E) had 1:5 neutralizing antibody (NAb) titers which is considered negative but may have impacted the efficiency of the gene transfer. When the steroid tapering began, the LDL level among the AAV3B-AR2.08 group started returning to baseline. All four animals in the AAV3B-AR2.08 group had received a second biopsy and showed decreased vector GC in liver. See, RA3345 (M) vs. RA3345-d83 (i.e., RA3345 (M) at day 83) and RA3380 (F) vs. RA3380-d88 (i.e., RA3380 (F) at day 88) in FIG. 10A.

### B. Comparison of the AAV Capsids and the hLDLR Expression Cassettes

Each of the six rAAV described in above were tested on four NHPs. One male and one female NHP received 2.5×10¹³ GC/kg (noted as “high” in the drawings, and also referred to as the “high dose” or “higher dose”) or 7.5×10¹² GC/kg (which is referred to as the “low dose” or “lower dose”).

Starting on the day of rAAV administration (day 0), animals received Prednisolone at 1 mg/kg body weight/day orally every day for 8 weeks. Animals were then tapered off Prednisolone by gradual reduction of daily dose.

Livers were biopsied on day 18 and a full necropsy was performed at four months post the rAAV administration (day 120). Clinical pathology, levels of cytokines, complements, lipids and T cell responses were also monitored.

### C. Comparison Between AAV8 with AAV3B.AR2.16 Using rAAV Containing the Native hLDLR Coding Sequence

The AAV capsids of AAV8 and AAV3B.AR2.16 were compared using the PI intron and the wild type hLDLR coding sequence.

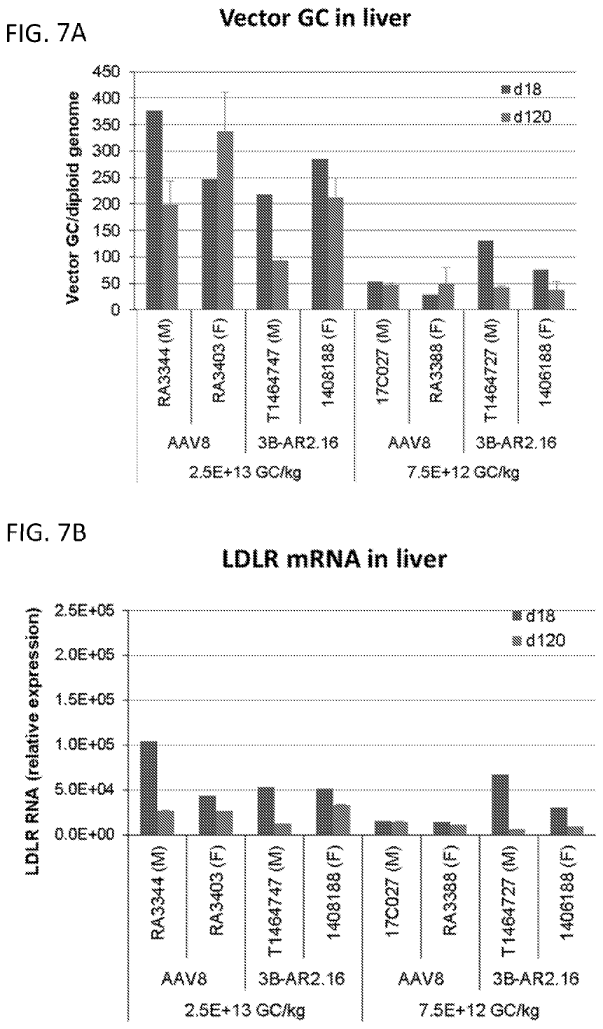

Experimental results showed that the rAAV particles successfully delivered the vector genome to liver. Briefly, liver samples from the biopsy on day 18 as well as the necropsy on day 120 were evaluated. Genome copies (GC) of the vector genome were normalized by diploid genome and plotted in FIG. 7A. Additionally, correlated LDLR mRNA relative expression was plotted (FIG. 7B). A dose dependence was observed, i.e., a higher dose results in more copies of vector in a cell. Although a slight decrease over time in the vector copies was observed in most of the animals, on day 120, the vector genome was not eliminated in any of the animal, suggesting a long-term effect of the rAAV treatment.

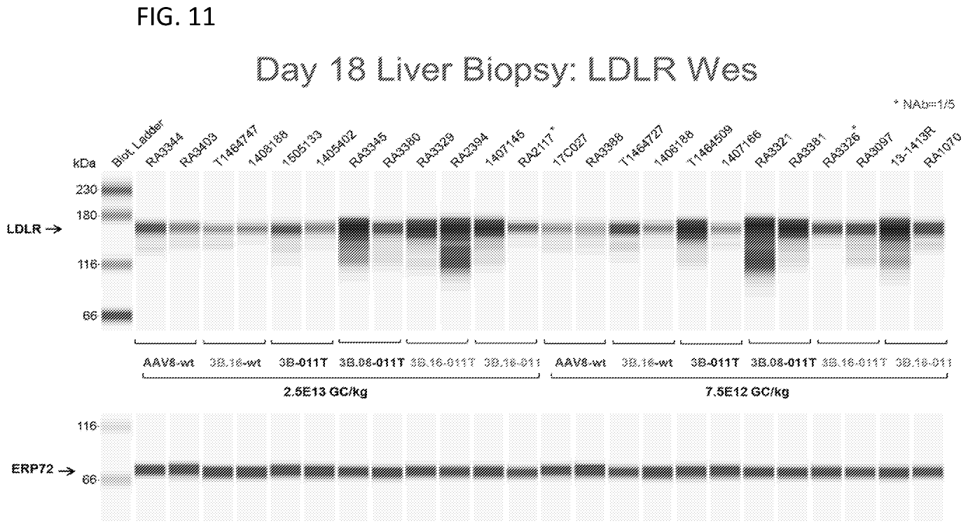

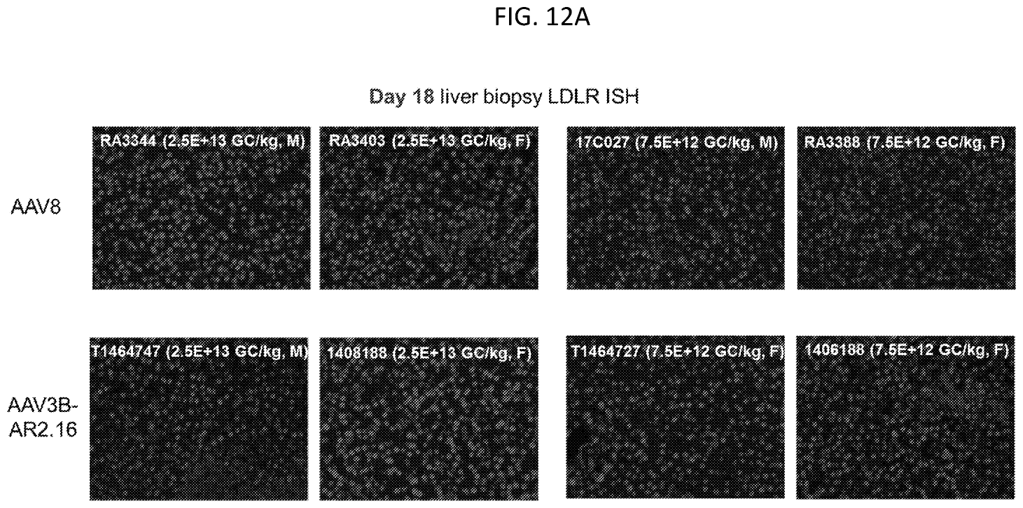

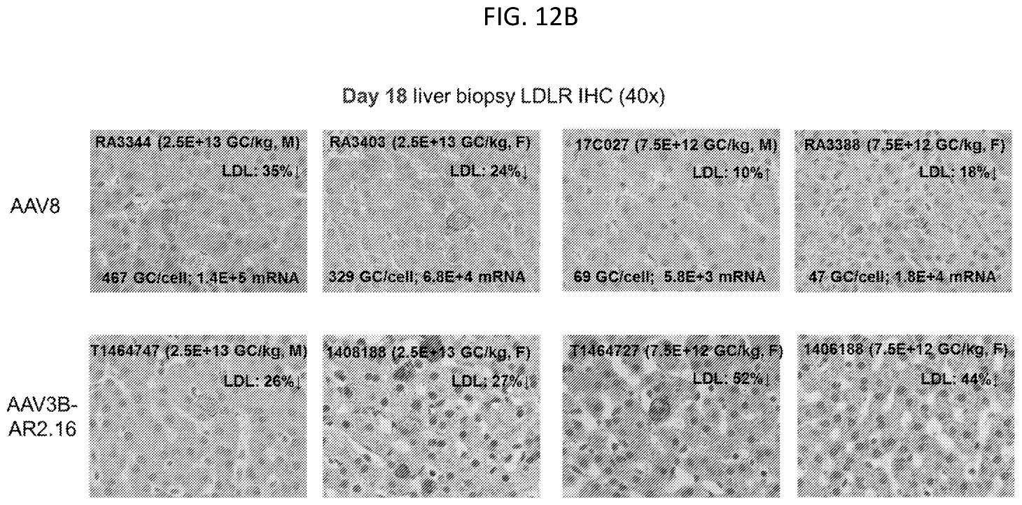

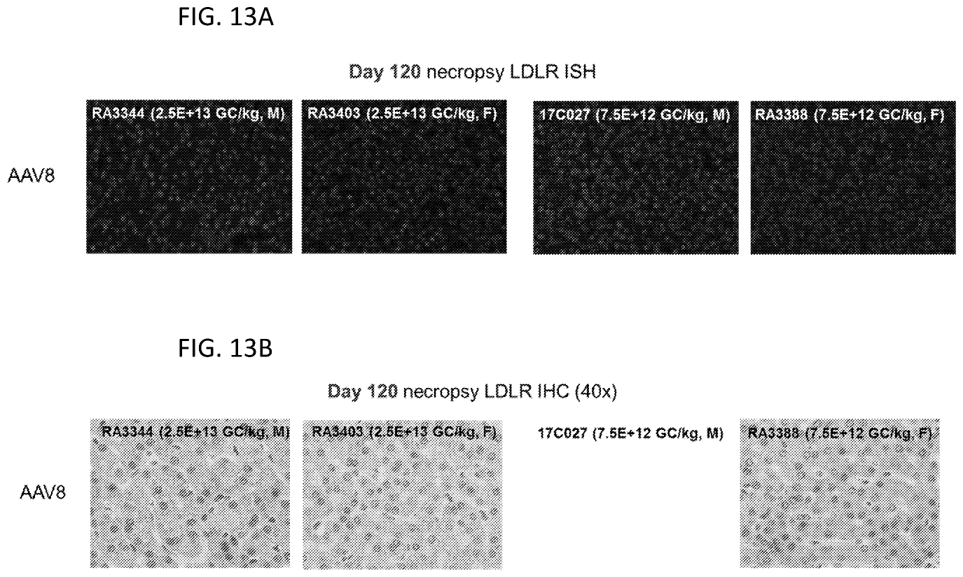

Further, a robust expression of the hLDLR protein was found in liver on day 18 post the AAV particle administration shown by western blot (WB), in situ hybridization (ISH) and immunohistochemistry (IHC). See, FIG. 11, FIG. 12A and FIG. 12B. On day 120, the expression level of liver LDLR protein was reduced in the animals treated with the AAV8 particles shown by WB, ISH and IHC as well as in the animals treated with high dose of the AAV3B.AR2.16 particles shown by WB. Still, LDLR expression in liver was observed even on day 120.

The low dose of the AAV8 particle did not lead to a significant LDL reduction upon administration. The male animal identified as RA3344 that was treated with the high dose of the AAV8 particle showed an LDL level reduced to a quarter of the starting level on day 0, while the female animal identified as RA3403 had no significant change in its LDL level. (FIG. 6A). However, both doses of the AAV3B.AR2.16 particle demonstrated its effectiveness shown by a significant reduction in the LDL level upon treatment (FIG. 6C), suggesting the AAV3B.AR2.16 capsid is more effective compared to the AAV8 for delivery to liver cells.

Potential toxicity to the liver was further evaluated via measuring ALT and AST levels with and without steroid. (FIG. 6B and FIG. 6D. A transient increase after injection was observed in the of the female animal and in the AST level of both animals treated with high dose of the AAV3B.AR2.16 particle. A male animal treated with the high dose of the AAV8 particle also showed a similar increase in its AST level. However, all ALT and AST levels returned to normal, indicating that no long-term liver damage occurred.

### D. Comparison of the Three hLDLR Expression Cassettes

Studies were performed to evaluate the three hLDLR expression cassettes (“hLDLR” vs. “hLDLR011” vs. “hLDLR011.triple”). The AAV3B.AR2.16 capsid was used to deliver a vector genome comprising one of the expression cassettes.

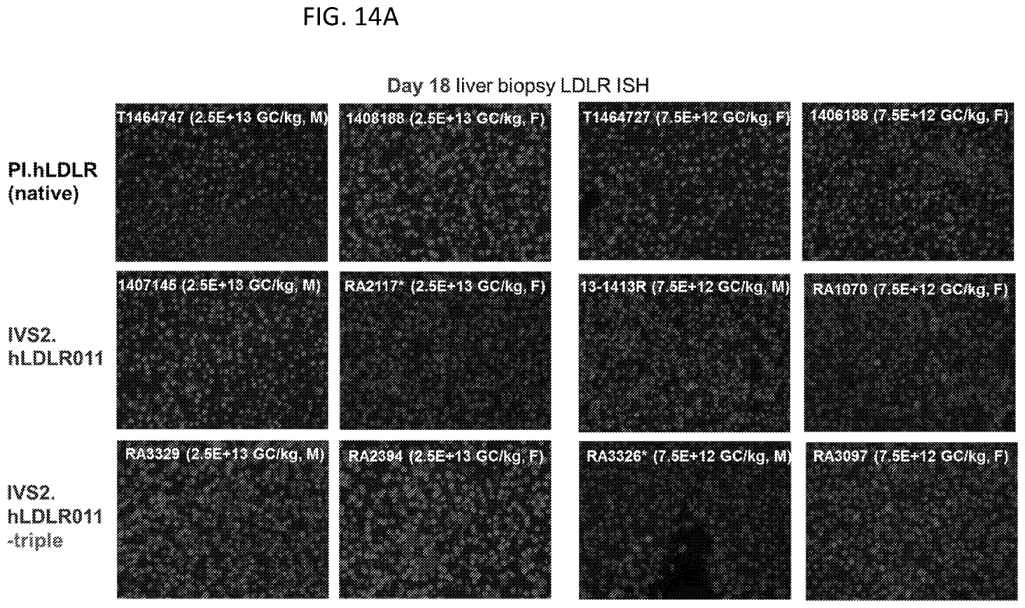

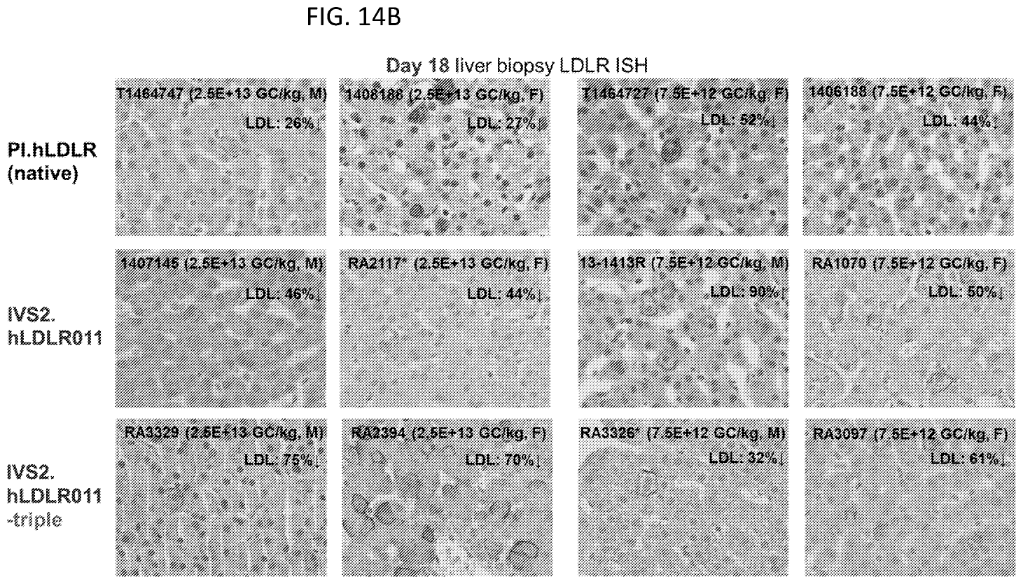

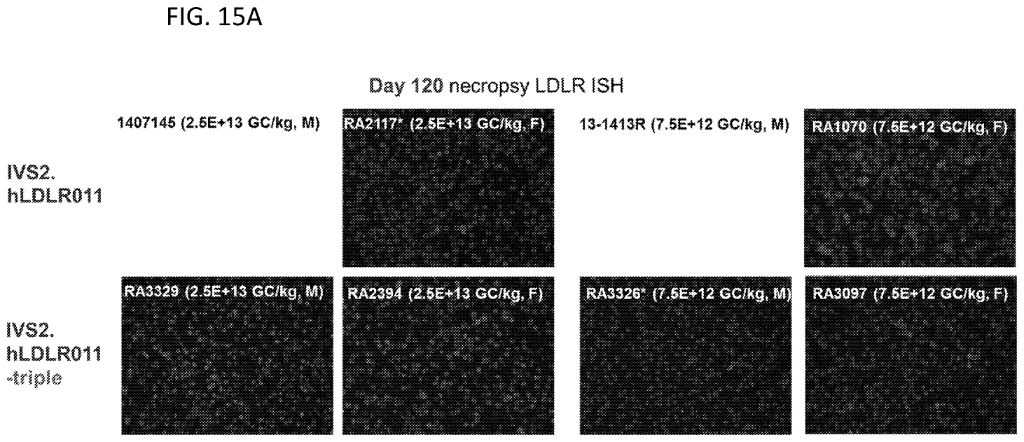

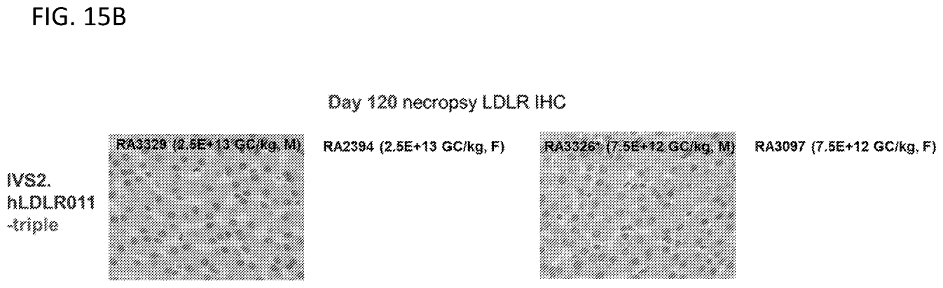

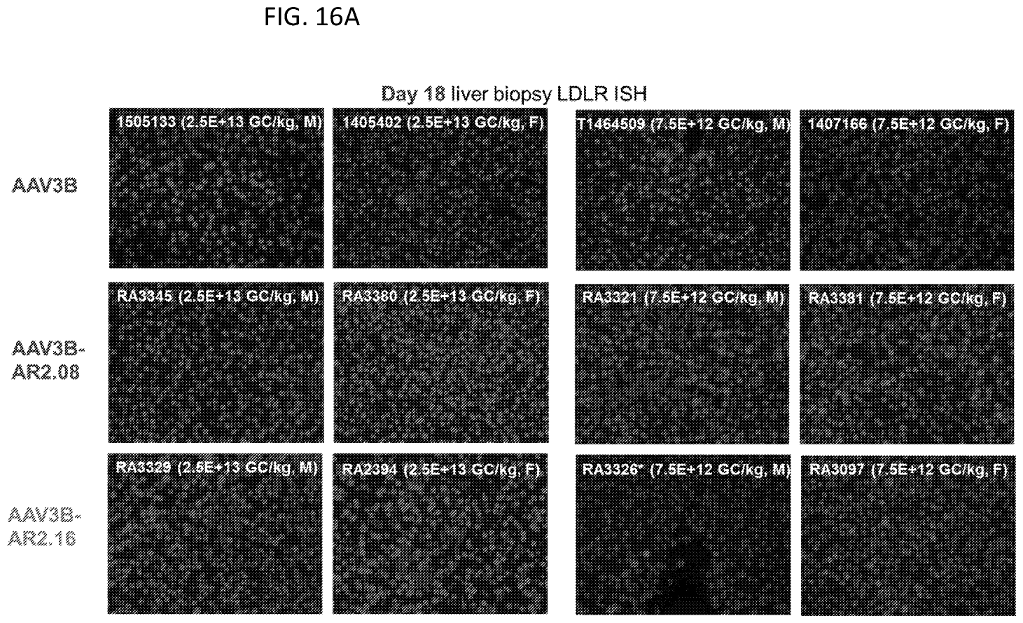

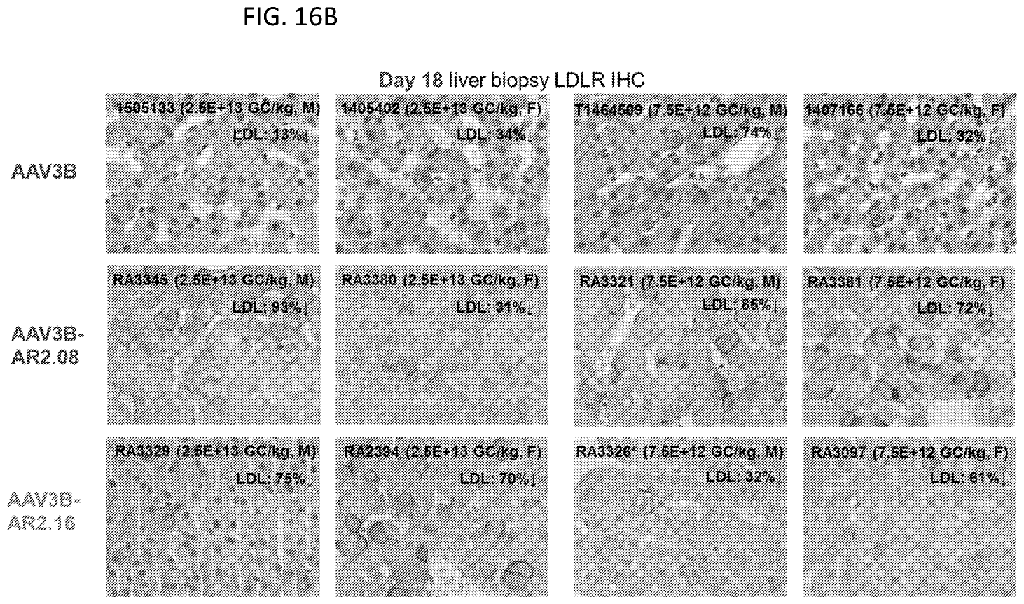

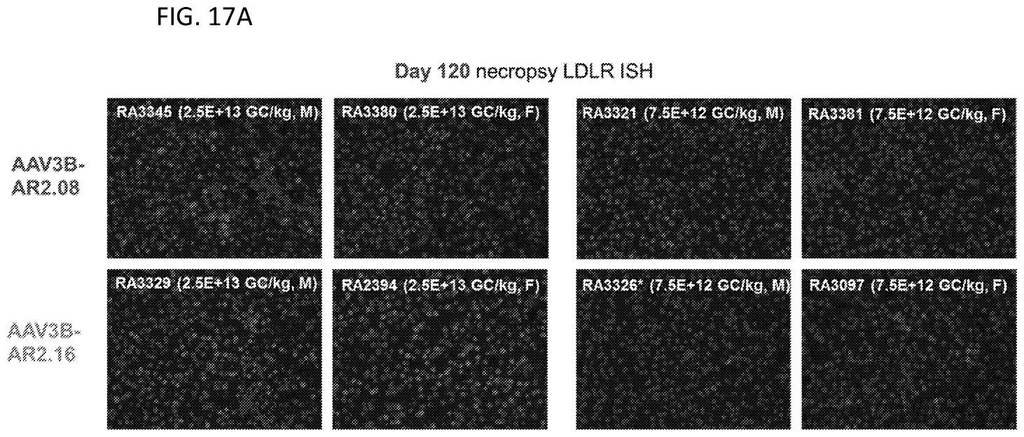

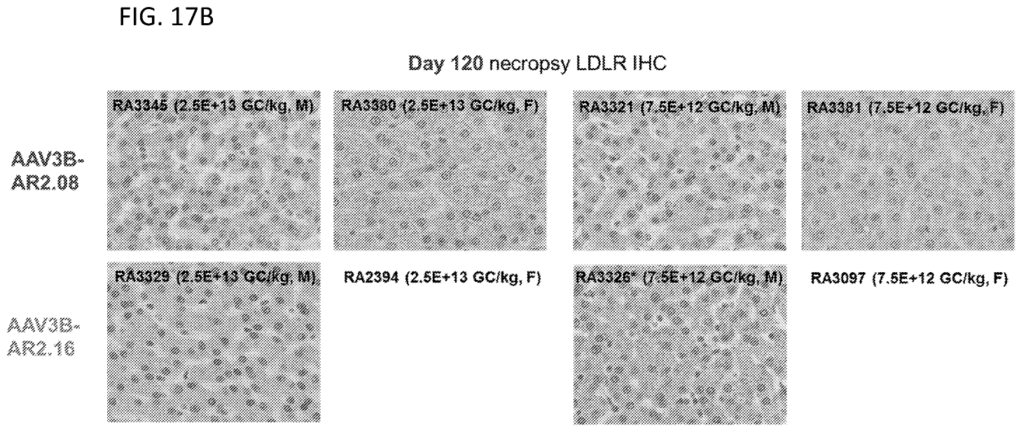

Expression of the hLDLR protein were identified in the liver samples of all groups biopsied on day 18 (FIG. 14A and FIG. 14B) shown via both ISH and IHC. The IHC images were provided with the corresponding LDL levels noted in the corner, showing a positive correlation between the LDLR expression level and the reduction in LDL. More results relating to the LDL level are plotted in FIG. 8E. At the higher dose, compared to the 75% reduction achieved by the wild-type LDLR coding sequence, both of the engineered sequences provided close to a 100% reduction upon administration. The lower dose of the rAAV.hLDLR011 particle resulted in an about 75% reduction and an about 100% reduction in LDL, while the lower dose of the rAAVhLDLR011.triple particle resulted in an about 50% reduction and an about 80% reduction. The data suggested that the engineered hLDLR coding sequences are better candidates in treating hypercholesterolemia compared to the wild-type sequence.

With respect to liver toxicity, advantages of the engineered hLDLR coding sequences over the wild type sequence were observed. A comparably low and stable level of ALT as well as AST was shown in both low and high dose groups of the animals treated with rAAV.hLDLR011 or rAAV.hLDLR011.triple particles. However, high dose of the rAAV particle comprising the wild type hLDLR sequence lead to a transient increase upon administration in the ALT level in one animal and in the AST levels in both animals.

Additionally, PCSK9 levels were also investigated for animals treated with AAV8.hLDLR, or AAV3B.AR2.08.hLDLR011.triple, or AAV3B.AR1.16.hLDLR011.triple. The LDL level of an animal treated with the AAV8.hLDLR particle followed the change in the PCSK9 level. See, for example, the animal identified as 17C027 from day 14 to day 42. However, such pattern was not found in the other two groups treated with AAV.hLDLR011.triple particles. See, for example, the animal identified as RA33289 from day 14 to day 28. The data suggest beneficial effects of the AAV.hLDLR011.triple particles. Additionally, a decline in the PCSK9 level up rAAV administration was observed, suggesting that PSCK9 responds to the decreased LDL level and/or the increased LDLR level due to the rAAV injection, and thus may play a negative role in reducing the LDL level if not controlled properly.

### E. Comparison of AAV3B Capsid with Two AAV3B Variants (all Containing the hLDLR011. Triple Coding Sequence)

The two tested AAV3B variants (i.e., AAV3B.AR2.08 and AAV3B.AR2.16) were further compared to the original AAV3B capsid via using the rAAV particles comprising the hLDLR011.triple coding sequence (see, e.g., FIG. 8A-FIG. 8F).

The LDLR expression was observed in all treated animals. Compared to the AAV3B particle, the AAV3B variant particles showed better effects in reducing the LDL level (FIG. 8C and FIG. 8E). For example, the LDL in the variant groups reached at a lower level upon treatment and stayed below the pre-treatment level for longer time. Additionally, the ALT level elevated in a sustained manner in the animals treated with the AAV3B particles while the AAV3B variants groups only showed a temporary increase. These results lead to a conclusion that the AAV3B variants are more advantageous over the original AAV3B capsid from the perspective of efficacy as well as safety.

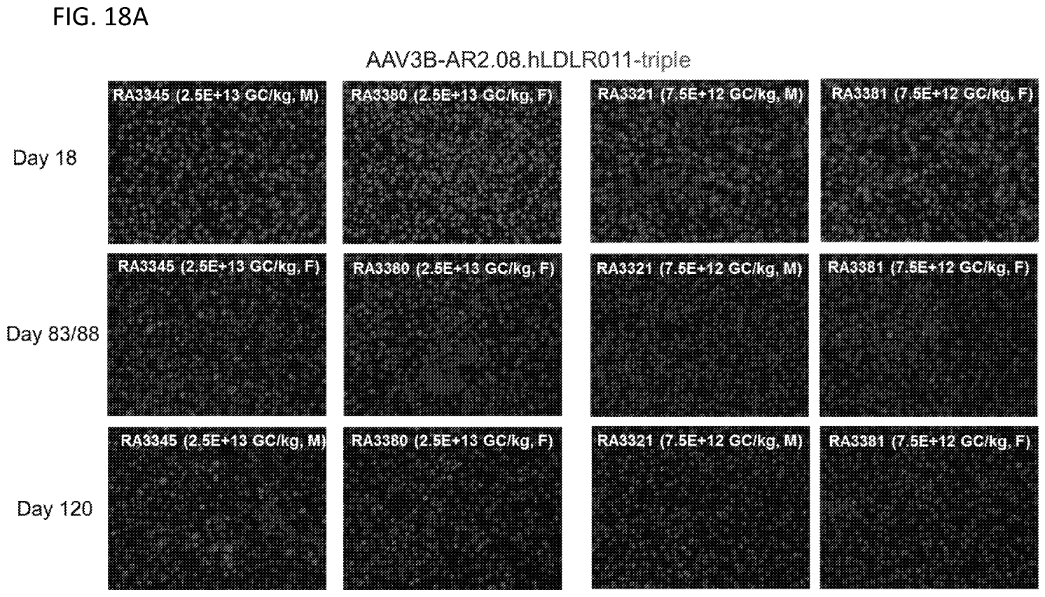

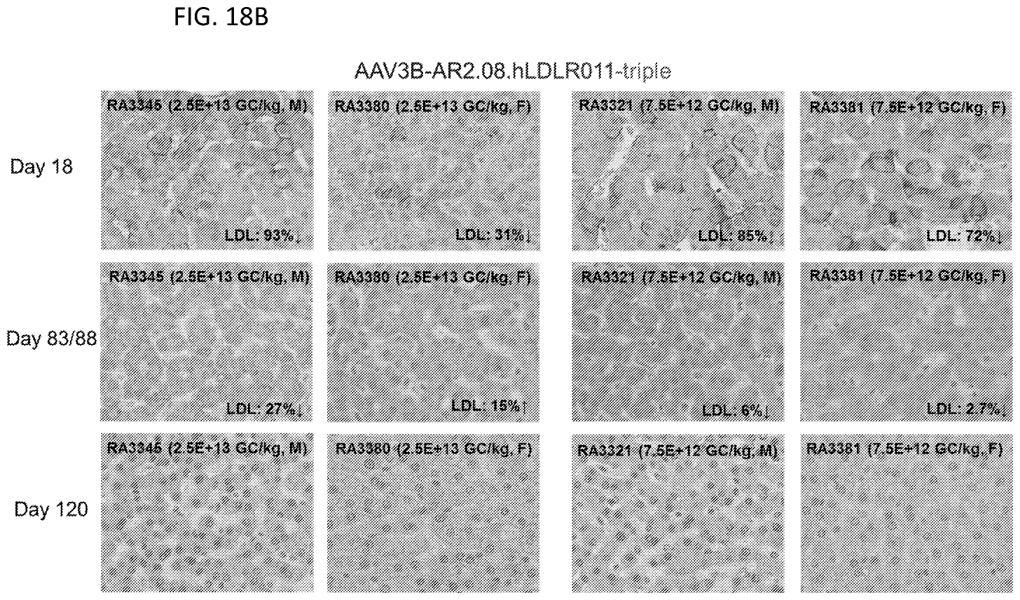

Interestingly, despite an initial response following administration, the ALT level and the LDL level shared a similar trend in the animals injected with AAV3B.AR2.08.hLDLR011.triple (See, FIG. 8D). Moreover, FIG. 18A and FIG. 18B provide ISH and IHC results for LDLR protein expression on day 18, day 83/88, and day 120. A gradual reduction was found, suggesting a clearing mechanism of the hLDLR expression cassette and/or the hLDLR expressing cells.

### F. Time Course of LDLR Expression in DKO Mouse Liver

A double knockout LDLR^{-/-} Apobec^{-/-} mouse model (DKO mouse) of homozygous FH (HoFH) was established. We evaluated IVS.hLDLR011-triple vector for sustainable expression over a period of 120 days. Male DKO mice received an IV administration of AAV8.IVS.hLDLR011-triple vector at a high dose of 7.5×10¹² GC/kg. A stable reduction of LDL-C levels in the serum is observed at multiple time points after the gene transfer. Liver tissues (n=5 mice/time point) were harvested on day 1, 3, 7, 14 and 120 after vector infusion and collected serum samples. Transduction efficiency of hepatocytes measured by PCR analysis revealed diploid vector genome copies per cell at day 1 that decreased two-fold at different time points and stable transgene expression (hLDLR mRNA) at different time points. Compared to day 1, hLDLR protein expression was 2-3 fold higher at days 3, 7, 14 and 120 (relative expression was analyzed by western blot). IHC staining and in situ hybridization analyses of liver showed a high level of expression of hLDLR at different time points.

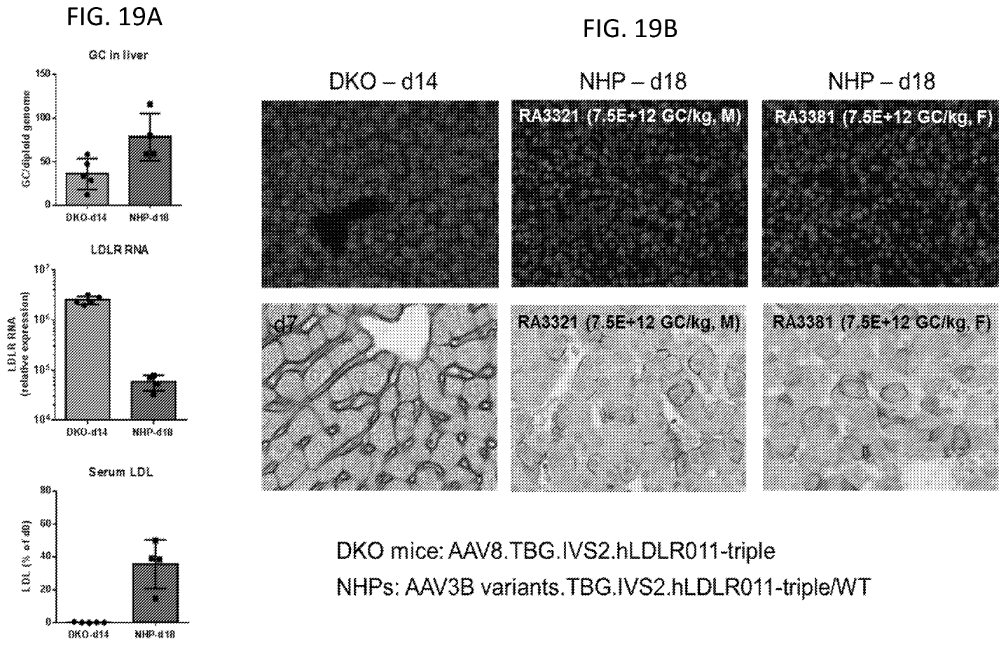

Despite the observation of the hLDLR expression cassette being gradually removed, a sustained hLDLR production (in terms of both RNA and protein levels, along with low LDL level) was revealed over the entire observation period until day 120 post rAAV administration. These results were then compared with NHPs administrated with the low dose of the particles using the AAV3B variant vectors. See, FIG. 19A-FIG. 19B.

### Example 5: Comparison with Other AAV Vectors

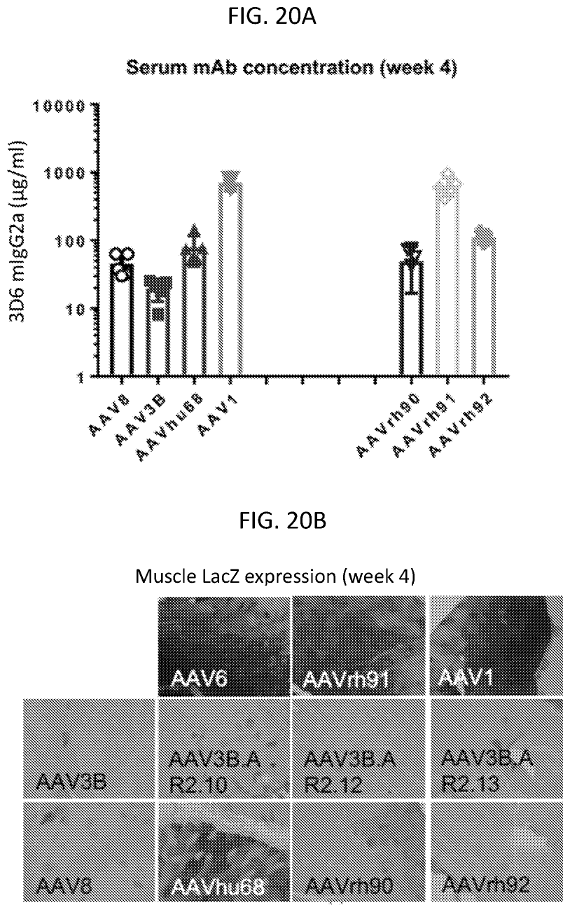

FIG. 20A and FIG. 20B show a comparison of muscle transduction and secreted protein levels in serum following IM delivery of multiple capsids. Vector expressing mAb from muscle selective promoter or LacZ was delivered IM. The data show that AAVrh91 achieves similar muscle transduction to AAV1 and AAV6 but has higher yields.

### Sequence Listing Free Text

The following information is provided for sequences containing free text under numeric identifier <223>.

| SEQ ID NO: | Free Text under <223> |
|---|---|
| 1 | <223> synthetic construct |
| 2 | <223> synthetic construct |
| 3 | <223> synthetic construct |
| 4 | <223> synthetic construct |
| 5 | <223> synthetic construct |
| 6 | <223> synthetic construct |
| 7 | <223> synthetic construct |
| 8 | <223> synthetic construct |
| 9 | <223> synthetic construct |
| 10 | <223> synthetic construct |
| 11 | <223> synthetic construct |
| 12 | <223> synthetic construct |
| 13 | <223> synthetic construct |
| 14 | <223> synthetic construct |
| 15 | <223> synthetic construct |
| 16 | <223> synthetic construct |
| 17 | <223> synthetic construct |
| 18 | <223> synthetic construct |
| 19 | <223> synthetic construct |
| 20 | <223> synthetic construct |
| 21 | <223> synthetic construct |
| 22 | <223> synthetic construct |
| 23 | <223> synthetic construct |
| 24 | <223> synthetic construct |
| 25 | <223> synthetic construct |
| 26 | <223> synthetic construct |
| 27 | <223> synthetic construct |
| 28 | <223> synthetic construct |
| 29 | <223> synthetic construct |
| 30 | <223> synthetic construct |
| 31 | <223> synthetic construct |
| 32 | <223> synthetic construct |
| 35 | <223> synthetic construct |
| 36 | <223> primer sequence |
| 37 | <223> primer sequence |
| 38 | <223> primer sequence |
| 39 | <223> primer sequence |
| 40 | <223> primer sequence |
| 41 | <223> miRNA target sequence |
| 42 | <223> miRNA target sequence |
| 43 | <223> miRNA target sequence |
| 44 | <223> miRNA target sequence |
| 45 | <223> synthetic construct |
| 46 | <223> synthetic construct |

All documents cited in this specification are incorporated herein by reference in their entireties. U.S. Provisional Patent Application No. 62/924,112, filed Oct. 21, 2019, and U.S. Provisional Patent Application No. 63/025,753, filed May 15, 2020, are incorporated by reference in their entireties, together with their sequence listings. The sequence listing filed herewith labeled “19-9050PCT_ST25.txt” and the sequences and text therein are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising a capsid and having packaged in said capsid a vector genome comprising a non-AAV nucleic acid sequence, wherein the capsid is selected from:
(a) an AAV3B.AR2.08 capsid which is produced from a nucleic acid sequence encoding an amino acid sequence of SEQ ID NO: 15 or an amino acid sequence having at least 95% identity thereto which retains amino acids at positions 582 to 594 of AAV3B.AR2.08 of SEQ ID NO: 15; or
(b) an AAV3B.AR2.16 capsid which is produced from a nucleic acid sequence encoding an amino acid sequence of SEQ ID NO: 29 or an amino acid sequence having at least 95% identity thereto which retains amino acids at positions 582 to 594 of AAV3B.AR2.16 of SEQ ID NO: 29.

2. (canceled)

3. The rAAV according to claim 1, wherein the AAV3B.AR2.08 capsid is encoded by a nucleic acid sequence of SEQ ID NO: 16 or a sequence at least 90% identical to SEQ ID NO: 16 encoding SEQ ID NO: 15.

4. (canceled)

5. The rAAV according to claim 1, wherein the AAV3B.AR2.16 capsid is encoded by a nucleic acid sequence of SEQ ID NO: 30 or a sequence at least 90% identical to SEQ ID NO: 30 encoding SEQ ID NO: 29.

6. The rAAV according to claim 1, wherein the vector genome comprises a tissue-specific promoter.

7. The rAAV according to claim 6, wherein the tissue-specific promoter is a liver-specific, muscle-specific or eye-specific promoter.

8. A recombinant adeno-associated virus (rAAV) comprising a capsid comprising a capsid protein
(i) having the sequence of AAV3B.AR2.01 (SEQ ID NO: 1), AAV3B.AR2.02 (SEQ ID NO: 3), AAV3B.AR2.03 (SEQ ID NO: 5), AAV3B.AR2.04 (SEQ ID NO: 7), AAV3B.AR2.05 (SEQ ID NO: 9), AAV3B.AR2.06 (SEQ ID NO: 11), AAV3B.AR2.07 (SEQ ID NO: 13), AAV3B.AR2.10 (SEQ ID NO: 17), AAV3B.AR2.11 (SEQ ID NO: 19), AAV3B.AR2.12 (SEQ ID NO: 21), AAV3B.AR2.13 (SEQ ID NO: 23), AAV3B.AR2.14 (SEQ ID NO: 25), AAV3B.AR2.15 (SEQ ID NO: 27), or AAV3B.AR2.17 (SEQ ID NO: 31); or
(ii) having a sequence encoded by the sequence of AAV3B.AR2.01 (SEQ ID NO: 2), AAV3B.AR2.02 (SEQ ID NO: 2), AAV3B.AR2.03 (SEQ ID NO: 6) AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), or AAV3B.AR2.17 (SEQ ID NO: 32), or a sequence sharing at least 90%, at least 95%, at least 97%, at least 98% or at least 99% identity with any of SEQ ID NO: 2, 4, 6, 8, 10, 12, 14, 16, 18, 20, 22, 24, 26, 28, 30 or 32;
and having packaged in said capsid a vector genome comprising a non-AAV nucleic acid sequence.

9. (canceled)

10. The rAAV according to claim 8, wherein the capsid protein is encoded by SEQ ID NO: 2, 4, 6, 8, 10, 12, 14, 18, 20, 22, 24, 26, 28, or 32.

11. The rAAV according to claim 1, wherein the vector genome comprises AAV inverted terminal repeats and a heterologous nucleic acid sequence operably linked to regulatory sequences which direct expression of a product encoded by the heterologous nucleic acid sequence in a target cell.

12. A recombinant adeno-associated virus (rAAV) comprising:
(A) an AAV capsid comprising:
(i) (1) AAV3B.AR2.01 capsid proteins comprising: a heterogeneous population of AAV3B.AR2.01 vp1 proteins selected from: vp1 proteins produced by expression from a nucleic acid sequence which encodes the predicted amino acid sequence of 1 to 736 of SEQ ID NO: 1, vp1 proteins produced from SEQ ID NO: 2, or vp1 proteins produced from a nucleic acid sequence at least 70% identical to SEQ ID NO: 2 which encodes the predicted amino acid sequence of 1 to 736 of SEQ ID NO: 1, a heterogeneous population of AAV3B.AR2.01 vp2 proteins selected from: vp2 proteins produced by expression from a nucleic acid sequence which encodes the predicted amino acid sequence of at least about amino acids 138 to 736 of SEQ ID NO: 1, vp2 proteins produced from a sequence comprising at least nucleotides 412 to 2211 of SEQ ID NO: 2, or vp2 proteins produced from a nucleic acid sequence at least 70% identical to at least nucleotides 412 to 2211 of SEQ ID NO: 2 which encodes the predicted amino acid sequence of at least about amino acids 138 to 736 of SEQ ID NO: 1, a heterogeneous population of AAV3B.AR2.01 vp3 proteins selected from: vp3 proteins produced by expression from a nucleic acid sequence which encodes the predicted amino acid sequence of at least about amino acids 203 to 736 of SEQ ID NO: 1, vp3 proteins produced from a sequence comprising at least nucleotides 607 to 2211 of SEQ ID NO: 2, or vp3 proteins produced from a nucleic acid sequence at least 70% identical to at least nucleotides 607 to 2211 of SEQ ID NO: 2 which encodes the predicted amino acid sequence of at least about amino acids 203 to 736 of SEQ ID NO: 1; and/or (2) a heterogeneous population of vp1 proteins which are the product of a nucleic acid sequence encoding the amino acid sequence of SEQ ID NO: 1, a heterogeneous population of vp2 proteins which are the product of a nucleic acid sequence encoding the amino acid sequence of at least about amino acids 138 to 736 of SEQ ID NO: 1, and a heterogeneous population of vp3 proteins which are the product of a nucleic acid sequence encoding at least amino acids 203 to 736 of SEQ ID NO: 1, wherein: the vp1, vp2 and vp3 proteins contain subpopulations with amino acid modifications comprising at least two highly deamidated asparagines (N) in asparagine-glycine pairs in SEQ ID NO: 1 and optionally further comprising subpopulations comprising other deamidated amino acids, wherein the deamidation results in an amino acid change;
(ii) (1) AAV3B.AR2.02 capsid proteins comprising: a heterogeneous population of AAV3B.AR2.02 vp1 proteins selected from: vp1 proteins produced by expression from a nucleic acid sequence which encodes the predicted amino acid sequence of 1 to 736 of SEQ ID NO: 3, vp1 proteins produced from SEQ ID NO: 4, or vp1 proteins produced from a nucleic acid sequence at least 70% identical to SEQ ID NO: 4 which encodes the predicted amino acid sequence of 1 to 736 of SEQ ID NO: 3, a heterogeneous population of AAV3B.AR2.02 vp2 proteins selected from: vp2 proteins produced by expression from a nucleic acid sequence which encodes the predicted amino acid sequence of at least about amino acids 138 to 736 of SEQ ID NO: 3, vp2 proteins produced from a sequence comprising at least nucleotides 412 to 2211 of SEQ ID NO: 4, or vp2 proteins produced from a nucleic acid sequence at least 70% identical to at least nucleotides 412 to 2211 of SEQ ID NO: 4 which encodes the predicted amino acid sequence of at least about amino acids 138 to 736 of SEQ ID NO: 3, a heterogeneous population of AAV3B.AR2.02 vp3 proteins selected from: vp3 proteins produced by expression from a nucleic acid sequence which encodes the predicted amino acid sequence of at least about amino acids 203 to 736 of SEQ ID NO: 3, vp3 proteins produced from a sequence comprising at least nucleotides 607 to 2211 of SEQ ID NO: 4, or vp3 proteins produced from a nucleic acid sequence at least 70% identical to at least nucleotides 607 to 2211 of SEQ ID NO: 4 which encodes the predicted amino acid sequence of at least about amino acids 203 to 736 of SEQ ID NO: 3; and/or (2) a heterogeneous population of vp1 proteins which are the product of a nucleic acid sequence encoding the amino acid sequence of SEQ ID NO: 3, a heterogeneous population of vp2 proteins which are the product of a nucleic acid sequence encoding the amino acid sequence of at least about amino acids 138 to 736 of SEQ ID NO: 3, and a heterogeneous population of vp3 proteins which are the product of a nucleic acid sequence encoding at least amino acids 203 to 736 of SEQ ID NO: 3, wherein: the vp1, vp2 and vp3 proteins contain subpopulations with amino acid modifications comprising at least two highly deamidated asparagines (N) in asparagine-glycine pairs in SEQ ID NO: 3 and optionally further comprising subpopulations comprising other deamidated amino acids, wherein the deamidation results in an amino acid change:
(iii) (1) AAV3B.AR2.03 capsid proteins comprising: a heterogeneous population of AAV3B.AR2.03 vp1 proteins selected from: vp1 proteins produced by expression from a nucleic acid sequence which encodes the predicted amino acid sequence of 1 to 736 of SEQ ID NO: 5, vp1 proteins produced from SEQ ID NO: 6, or vp1 proteins produced from a nucleic acid sequence at least 70% identical to SEQ ID NO: 6 which encodes the predicted amino acid sequence of 1 to 736 of SEQ ID NO: 5, a heterogeneous population of AAV3B.AR2.03 vp2 proteins selected from: vp2 proteins produced by expression from a nucleic acid sequence which encodes the predicted amino acid sequence of at least about amino acids 138 to 736 of SEQ ID NO: 5, vp2 proteins produced from a sequence comprising at least nucleotides 412 to 2211 of SEQ ID NO: 6, or vp2 proteins produced from a nucleic acid sequence at least 70% identical to at least nucleotides 412 to 2211 of SEQ ID NO: 6 which encodes the predicted amino acid sequence of at least about amino acids 138 to 736 of SEQ ID NO: 5, a heterogeneous population of AAV3B.AR2.03 vp3 proteins selected from: vp3 proteins produced by expression from a nucleic acid sequence which encodes the predicted amino acid sequence of at least about amino acids 203 to 736 of SEQ ID NO: 5, vp3 proteins produced from a sequence comprising at least nucleotides 607 to 2211 of SEQ ID NO: 6, or vp3 proteins produced from a nucleic acid sequence at least 70% identical to at least nucleotides 607 to 2211 of SEQ ID NO: 6 which encodes the predicted amino acid sequence of at least about amino acids 203 to 736 of SEQ ID NO: 5; and/or (2) a heterogeneous population of vp1 proteins which are the product of a nucleic acid sequence encoding the amino acid sequence of SEQ ID NO: 5, a heterogeneous population of vp2 proteins which are the product of a nucleic acid sequence encoding the amino acid sequence of at least about amino acids 138 to 736 of SEQ ID NO: 5, and a heterogeneous population of vp3 proteins which are the product of a nucleic acid sequence encoding at least amino acids 203 to 736 of SEQ ID NO: 5, wherein: the vp1, vp2 and vp3 proteins contain subpopulations with amino acid modifications comprising at least two highly deamidated asparagines (N) in asparagine-glycine pairs in SEQ ID NO: 5 and optionally further comprising subpopulations comprising other deamidated amino acids, wherein the deamidation results in an amino acid change;
(iv) (1) AAV3B.AR2.04 capsid proteins comprising: a heterogeneous population of AAV3B.AR2.04 vp1 proteins selected from: vp1 proteins produced by expression from a nucleic acid sequence which encodes the predicted amino acid sequence of 1 to 736 of SEQ ID NO: 7, vp1 proteins produced from SEQ ID NO: 8, or vp1 proteins produced from a nucleic acid sequence at least 70% identical to SEQ ID NO: 8 which encodes the predicted amino acid sequence of 1 to 736 of SEQ ID NO: 7, a heterogeneous population of AAV3B.AR2.04 vp2 proteins selected from: vp2 proteins produced by expression from a nucleic acid sequence which encodes the predicted amino acid sequence of at least about amino acids 138 to 736 of SEQ ID NO: 7, vp2 proteins produced from a sequence comprising at least nucleotides 412 to 2211 of SEQ ID NO: 8, or vp2 proteins produced from a nucleic acid sequence at least 70% identical to at least nucleotides 412 to 2211 of SEQ ID NO: 8 which encodes the predicted amino acid sequence of at least about amino acids 138 to 736 of SEQ ID NO: 7, a heterogeneous population of AAV3B.AR2.04 vp3 proteins selected from: vp3 proteins produced by expression from a nucleic acid sequence which encodes the predicted amino acid sequence of at least about amino acids 203 to 736 of SEQ ID NO: 7, vp3 proteins produced from a sequence comprising at least nucleotides 607 to 2211 of SEQ ID NO: 8, or vp3 proteins produced from a nucleic acid sequence at least 70% identical to at least nucleotides 607 to 2211 of SEQ ID NO: 8 which encodes the predicted amino acid sequence of at least about amino acids 203 to 736 of SEQ ID NO: 7; and/or (2) a heterogeneous population of vp1 proteins which are the product of a nucleic acid sequence encoding the amino acid sequence of SEQ ID NO: 7, a heterogeneous population of vp2 proteins which are the product of a nucleic acid sequence encoding the amino acid sequence of at least about amino acids 138 to 736 of SEQ ID NO: 7, and a heterogeneous population of vp3 proteins which are the product of a nucleic acid sequence encoding at least amino acids 203 to 736 of SEQ ID NO: 7, wherein: the vp1, vp2 and vp3 proteins contain subpopulations with amino acid modifications comprising at least two highly deamidated asparagines (N) in asparagine-glycine pairs in SEQ ID NO: 7 and optionally further comprising subpopulations comprising other deamidated amino acids, wherein the deamidation results in an amino acid change;
(v) (1) AAV3B.AR2.05 capsid proteins comprising: a heterogeneous population of AAV3B.AR2.05 vp1 proteins selected from: vp1 proteins produced by expression from a nucleic acid sequence which encodes the predicted amino acid sequence of 1 to 736 of SEQ ID NO: 9, vp1 proteins produced from SEQ ID NO: 10, or vp1 proteins produced from a nucleic acid sequence at least 70% identical to SEQ ID NO: 10 which encodes the predicted amino acid sequence of 1 to 736 of SEQ ID NO: 9, a heterogeneous population of AAV3B.AR2.05 vp2 proteins selected from: vp2 proteins produced by expression from a nucleic acid sequence which encodes the predicted amino acid sequence of at least about amino acids 138 to 736 of SEQ ID NO: 9, vp2 proteins produced from a sequence comprising at least nucleotides 412 to 2211 of SEQ ID NO: 10, or vp2 proteins produced from a nucleic acid sequence at least 70% identical to at least nucleotides 412 to 2211 of SEQ ID NO: 10 which encodes the predicted amino acid sequence of at least about amino acids 138 to 736 of SEQ ID NO: 9, a heterogeneous population of AAV3B.AR2.05 vp3 proteins selected from: vp3 proteins produced by expression from a nucleic acid sequence which encodes the predicted amino acid sequence of at least about amino acids 203 to 736 of SEQ ID NO: 9, vp3 proteins produced from a sequence comprising at least nucleotides 607 to 2211 of SEQ ID NO: 10, or vp3 proteins produced from a nucleic acid sequence at least 70% identical to at least nucleotides 607 to 2211 of SEQ ID NO: 10 which encodes the predicted amino acid sequence of at least about amino acids 203 to 736 of SEQ ID NO: 9; and/or (2) a heterogeneous population of vp1 proteins which are the product of a nucleic acid sequence encoding the amino acid sequence of SEQ ID NO: 9, a heterogeneous population of vp2 proteins which are the product of a nucleic acid sequence encoding the amino acid sequence of at least about amino acids 138 to 736 of SEQ ID NO: 9, and a heterogeneous population of vp3 proteins which are the product of a nucleic acid sequence encoding at least amino acids 203 to 736 of SEQ ID NO: 9, wherein: the vp1, vp2 and vp3 proteins contain subpopulations with amino acid modifications comprising at least two highly deamidated asparagines (N) in asparagine-glycine pairs in SEQ ID NO: 9 and optionally further comprising subpopulations comprising other deamidated amino acids, wherein the deamidation results in an amino acid change;
(vi) (1) AAV3B.AR2.06 capsid proteins comprising: a heterogeneous population of AAV3B.AR2.06 vp1 proteins selected from: vp1 proteins produced by expression from a nucleic acid sequence which encodes the predicted amino acid sequence of 1 to 736 of SEQ ID NO: 11, vp1 proteins produced from SEQ ID NO: 12, or vp1 proteins produced from a nucleic acid sequence at least 70% identical to SEQ ID NO: 12 which encodes the predicted amino acid sequence of 1 to 736 of SEQ ID NO: 11, a heterogeneous population of AAV3B.AR2.06 vp2 proteins selected from: vp2 proteins produced by expression from a nucleic acid sequence which encodes the predicted amino acid sequence of at least about amino acids 138 to 736 of SEQ ID NO: 11, vp2 proteins produced from a sequence comprising at least nucleotides 412 to 2211 of SEQ ID NO: 12, or vp2 proteins produced from a nucleic acid sequence at least 70% identical to at least nucleotides 412 to 2211 of SEQ ID NO: 12 which encodes the predicted amino acid sequence of at least about amino acids 138 to 736 of SEQ ID NO: 11, a heterogeneous population of AAV3B.AR2.06 vp3 proteins selected from: vp3 proteins produced by expression from a nucleic acid sequence which encodes the predicted amino acid sequence of at least about amino acids 203 to 736 of SEQ ID NO: 11, vp3 proteins produced from a sequence comprising at least nucleotides 607 to 2211 of SEQ ID NO: 12, or vp3 proteins produced from a nucleic acid sequence at least 70% identical to at least nucleotides 607 to 2211 of SEQ ID NO: 12 which encodes the predicted amino acid sequence of at least about amino acids 203 to 736 of SEQ ID NO: 11; and/or (2) a heterogeneous population of vp1 proteins which are the product of a nucleic acid sequence encoding the amino acid sequence of SEQ ID NO: 11, a heterogeneous population of vp2 proteins which are the product of a nucleic acid sequence encoding the amino acid sequence of at least about amino acids 138 to 736 of SEQ ID NO: 11, and a heterogeneous population of vp3 proteins which are the product of a nucleic acid sequence encoding at least amino acids 203 to 736 of SEQ ID NO: 11, wherein: the vp1, vp2 and vp3 proteins contain subpopulations with amino acid modifications comprising at least two highly deamidated asparagines (N) in asparagine-glycine pairs in SEQ ID NO: 11 and optionally further comprising subpopulations comprising other deamidated amino acids, wherein the deamidation results in an amino acid change;
(vii) (1) AAV3B.AR2.07 capsid proteins comprising: a heterogeneous population of AAV3B.AR2.07 vp1 proteins selected from: vp1 proteins produced by expression from a nucleic acid sequence which encodes the predicted amino acid sequence of 1 to 736 of SEQ ID NO: 13, vp1 proteins produced from SEQ ID NO: 14, or vp1 proteins produced from a nucleic acid sequence at least 70% identical to SEQ ID NO: 14 which encodes the predicted amino acid sequence of 1 to 736 of SEQ ID NO: 13, a heterogeneous population of AAV3B.AR2.07 vp2 proteins selected from: vp2 proteins produced by expression from a nucleic acid sequence which encodes the predicted amino acid sequence of at least about amino acids 138 to 736 of SEQ ID NO: 13, vp2 proteins produced from a sequence comprising at least nucleotides 412 to 2211 of SEQ ID NO: 14, or vp2 proteins produced from a nucleic acid sequence at least 70% identical to at least nucleotides 412 to 2211 of SEQ ID NO: 14 which encodes the predicted amino acid sequence of at least about amino acids 138 to 736 of SEQ ID NO: 13, a heterogeneous population of AAV3B.AR2.07 vp3 proteins selected from: vp3 proteins produced by expression from a nucleic acid sequence which encodes the predicted amino acid sequence of at least about amino acids 203 to 736 of SEQ ID NO: 13, vp3 proteins produced from a sequence comprising at least nucleotides 607 to 2211 of SEQ ID NO: 14, or vp3 proteins produced from a nucleic acid sequence at least 70% identical to at least nucleotides 607 to 2211 of SEQ ID NO: 14 which encodes the predicted amino acid sequence of at least about amino acids 203 to 736 of SEQ ID NO: 13; and/or (2) a heterogeneous population of vp1 proteins which are the product of a nucleic acid sequence encoding the amino acid sequence of SEQ ID NO: 13, a heterogeneous population of vp2 proteins which are the product of a nucleic acid sequence encoding the amino acid sequence of at least about amino acids 138 to 736 of SEQ ID NO: 13, and a heterogeneous population of vp3 proteins which are the product of a nucleic acid sequence encoding at least amino acids 203 to 736 of SEQ ID NO: 13, wherein: the vp1, vp2 and vp3 proteins contain subpopulations with amino acid modifications comprising at least two highly deamidated asparagines (N) in asparagine-glycine pairs in SEQ ID NO: 13 and optionally further comprising subpopulations comprising other deamidated amino acids, wherein the deamidation results in an amino acid change;
(viii) (1) AAV3B.AR2.08 capsid proteins comprising: a heterogeneous population of AAV3B.AR2.08 vp1 proteins selected from: vp1 proteins produced by expression from a nucleic acid sequence which encodes the predicted amino acid sequence of 1 to 736 of SEQ ID NO: 15, vp1 proteins produced from SEQ ID NO: 16, or vp1 proteins produced from a nucleic acid sequence at least 70% identical to SEQ ID NO: 16 which encodes the predicted amino acid sequence of 1 to 736 of SEQ ID NO: 15, a heterogeneous population of AAV3B.AR2.08 vp2 proteins selected from: vp2 proteins produced by expression from a nucleic acid sequence which encodes the predicted amino acid sequence of at least about amino acids 138 to 736 of SEQ ID NO: 15, vp2 proteins produced from a sequence comprising at least nucleotides 412 to 2211 of SEQ ID NO: 16, or vp2 proteins produced from a nucleic acid sequence at least 70% identical to at least nucleotides 412 to 2211 of SEQ ID NO: 16 which encodes the predicted amino acid sequence of at least about amino acids 138 to 736 of SEQ ID NO: 15, a heterogeneous population of AAV3B.AR2.08 vp3 proteins selected from: vp3 proteins produced by expression from a nucleic acid sequence which encodes the predicted amino acid sequence of at least about amino acids 203 to 736 of SEQ ID NO: 15, vp3 proteins produced from a sequence comprising at least nucleotides 607 to 2211 of SEQ ID NO: 16, or vp3 proteins produced from a nucleic acid sequence at least 70% identical to at least nucleotides 607 to 2211 of SEQ ID NO: 16 which encodes the predicted amino acid sequence of at least about amino acids 203 to 736 of SEQ ID NO: 15; and/or (2) a heterogeneous population of vp1 proteins which are the product of a nucleic acid sequence encoding the amino acid sequence of SEQ ID NO: 15, a heterogeneous population of vp2 proteins which are the product of a nucleic acid sequence encoding the amino acid sequence of at least about amino acids 138 to 736 of SEQ ID NO: 15, and a heterogeneous population of vp3 proteins which are the product of a nucleic acid sequence encoding at least amino acids 203 to 736 of SEQ ID NO: 15, wherein: the vp1, vp2 and vp3 proteins contain subpopulations with amino acid modifications comprising at least two highly deamidated asparagines (N) in asparagine-glycine pairs in SEQ ID NO: 15 and optionally further comprising subpopulations comprising other deamidated amino acids, wherein the deamidation results in an amino acid change;
(ix) (1) AAV3B.AR2.10 capsid proteins comprising: a heterogeneous population of AAV3B.AR2.10 vp1 proteins selected from: vp1 proteins produced by expression from a nucleic acid sequence which encodes the predicted amino acid sequence of 1 to 736 of SEQ ID NO: 17, vp1 proteins produced from SEQ ID NO: 18, or vp1 proteins produced from a nucleic acid sequence at least 70% identical to SEQ ID NO: 18 which encodes the predicted amino acid sequence of 1 to 736 of SEQ ID NO: 17, a heterogeneous population of AAV3B.AR2.10 vp2 proteins selected from: vp2 proteins produced by expression from a nucleic acid sequence which encodes the predicted amino acid sequence of at least about amino acids 138 to 736 of SEQ ID NO: 17, vp2 proteins produced from a sequence comprising at least nucleotides 412 to 2211 of SEQ ID NO: 18, or vp2 proteins produced from a nucleic acid sequence at least 70% identical to at least nucleotides 412 to 2211 of SEQ ID NO: 18 which encodes the predicted amino acid sequence of at least about amino acids 138 to 736 of SEQ ID NO: 17, a heterogeneous population of AAV3B.AR2.10 vp3 proteins selected from: vp3 proteins produced by expression from a nucleic acid sequence which encodes the predicted amino acid sequence of at least about amino acids 203 to 736 of SEQ ID NO: 17, vp3 proteins produced from a sequence comprising at least nucleotides 607 to 2211 of SEQ ID NO: 18, or vp3 proteins produced from a nucleic acid sequence at least 70% identical to at least nucleotides 607 to 2211 of SEQ ID NO: 18 which encodes the predicted amino acid sequence of at least about amino acids 203 to 736 of SEQ ID NO: 17; and/or (2) a heterogeneous population of vp1 proteins which are the product of a nucleic acid sequence encoding the amino acid sequence of SEQ ID NO: 17, a heterogeneous population of vp2 proteins which are the product of a nucleic acid sequence encoding the amino acid sequence of at least about amino acids 138 to 736 of SEQ ID NO: 17, and a heterogeneous population of vp3 proteins which are the product of a nucleic acid sequence encoding at least amino acids 203 to 736 of SEQ ID NO: 17, wherein: the vp1, vp2 and vp3 proteins contain subpopulations with amino acid modifications comprising at least two highly deamidated asparagines (N) in asparagine-glycine pairs in SEQ ID NO: 17 and optionally further comprising subpopulations comprising other deamidated amino acids, wherein the deamidation results in an amino acid change;
(x) (1) AAV3B.AR2.11 capsid proteins comprising: a heterogeneous population of AAV3B.AR2.11 vp1 proteins selected from: vp1 proteins produced by expression from a nucleic acid sequence which encodes the predicted amino acid sequence of 1 to 736 of SEQ ID NO: 19, vp1 proteins produced from SEQ ID NO: 20, or vp1 proteins produced from a nucleic acid sequence at least 70% identical to SEQ ID NO: 20 which encodes the predicted amino acid sequence of 1 to 736 of SEQ ID NO: 19, a heterogeneous population of AAV3B.AR2.11 vp2 proteins selected from: vp2 proteins produced by expression from a nucleic acid sequence which encodes the predicted amino acid sequence of at least about amino acids 138 to 736 of SEQ ID NO: 19, vp2 proteins produced from a sequence comprising at least nucleotides 412 to 2211 of SEQ ID NO: 20, or vp2 proteins produced from a nucleic acid sequence at least 70% identical to at least nucleotides 412 to 2211 of SEQ ID NO: 20 which encodes the predicted amino acid sequence of at least about amino acids 138 to 736 of SEQ ID NO: 19, a heterogeneous population of AAV3B.AR2.11 vp3 proteins selected from: vp3 proteins produced by expression from a nucleic acid sequence which encodes the predicted amino acid sequence of at least about amino acids 203 to 736 of SEQ ID NO: 19, vp3 proteins produced from a sequence comprising at least nucleotides 607 to 2211 of SEQ ID NO: 20, or vp3 proteins produced from a nucleic acid sequence at least 70% identical to at least nucleotides 607 to 2211 of SEQ ID NO: 20 which encodes the predicted amino acid sequence of at least about amino acids 203 to 736 of SEQ ID NO: 19; and/or (2) a heterogeneous population of vp1 proteins which are the product of a nucleic acid sequence encoding the amino acid sequence of SEQ ID NO: 19, a heterogeneous population of vp2 proteins which are the product of a nucleic acid sequence encoding the amino acid sequence of at least about amino acids 138 to 736 of SEQ ID NO: 19, and a heterogeneous population of vp3 proteins which are the product of a nucleic acid sequence encoding at least amino acids 203 to 736 of SEQ ID NO: 19, wherein: the vp1, vp2 and vp3 proteins contain subpopulations with amino acid modifications comprising at least two highly deamidated asparagines (N) in asparagine-glycine pairs in SEQ ID NO: 19 and optionally further comprising subpopulations comprising other deamidated amino acids, wherein the deamidation results in an amino acid change;
(xi) (1) AAV3B.AR2.12 capsid proteins comprising: a heterogeneous population of AAV3B.AR2.12 vp1 proteins selected from: vp1 proteins produced by expression from a nucleic acid sequence which encodes the predicted amino acid sequence of 1 to 736 of SEQ ID NO: 21, vp1 proteins produced from SEQ ID NO: 22, or vp1 proteins produced from a nucleic acid sequence at least 70% identical to SEQ ID NO: 22 which encodes the predicted amino acid sequence of 1 to 736 of SEQ ID NO: 21, a heterogeneous population of AAV3B.AR2.12 vp2 proteins selected from: vp2 proteins produced by expression from a nucleic acid sequence which encodes the predicted amino acid sequence of at least about amino acids 138 to 736 of SEQ ID NO: 21, vp2 proteins produced from a sequence comprising at least nucleotides 412 to 2211 of SEQ ID NO: 22, or vp2 proteins produced from a nucleic acid sequence at least 70% identical to at least nucleotides 412 to 2211 of SEQ ID NO: 22 which encodes the predicted amino acid sequence of at least about amino acids 138 to 736 of SEQ ID NO: 21, a heterogeneous population of AAV3B.AR2.12 vp3 proteins selected from: vp3 proteins produced by expression from a nucleic acid sequence which encodes the predicted amino acid sequence of at least about amino acids 203 to 736 of SEQ ID NO: 21, vp3 proteins produced from a sequence comprising at least nucleotides 607 to 2211 of SEQ ID NO: 22, or vp3 proteins produced from a nucleic acid sequence at least 70% identical to at least nucleotides 607 to 2211 of SEQ ID NO: 22 which encodes the predicted amino acid sequence of at least about amino acids 203 to 736 of SEQ ID NO: 21; and/or (2) a heterogeneous population of vp1 proteins which are the product of a nucleic acid sequence encoding the amino acid sequence of SEQ ID NO: 21, a heterogeneous population of vp2 proteins which are the product of a nucleic acid sequence encoding the amino acid sequence of at least about amino acids 138 to 736 of SEQ ID NO: 21, and a heterogeneous population of vp3 proteins which are the product of a nucleic acid sequence encoding at least amino acids 203 to 736 of SEQ ID NO: 21, wherein: the vp1, vp2 and vp3 proteins contain subpopulations with amino acid modifications comprising at least two highly deamidated asparagines (N) in asparagine-glycine pairs in SEQ ID NO: 21 and optionally further comprising subpopulations comprising other deamidated amino acids, wherein the deamidation results in an amino acid change;
(xii) (1) AAV3B.AR2.13 capsid proteins comprising: a heterogeneous population of AAV3B.AR2.13 vp1 proteins selected from: vp1 proteins produced by expression from a nucleic acid sequence which encodes the predicted amino acid sequence of 1 to 736 of SEQ ID NO: 23, vp1 proteins produced from SEQ ID NO: 24, or vp1 proteins produced from a nucleic acid sequence at least 70% identical to SEQ ID NO: 24 which encodes the predicted amino acid sequence of 1 to 736 of SEQ ID NO: 23, a heterogeneous population of AAV3B.AR2.13 vp2 proteins selected from: vp2 proteins produced by expression from a nucleic acid sequence which encodes the predicted amino acid sequence of at least about amino acids 138 to 736 of SEQ ID NO: 23, vp2 proteins produced from a sequence comprising at least nucleotides 412 to 2211 of SEQ ID NO: 24, or vp2 proteins produced from a nucleic acid sequence at least 70% identical to at least nucleotides 412 to 2211 of SEQ ID NO: 24 which encodes the predicted amino acid sequence of at least about amino acids 138 to 736 of SEQ ID NO: 23, a heterogeneous population of AAV3B.AR2.13 vp3 proteins selected from: vp3 proteins produced by expression from a nucleic acid sequence which encodes the predicted amino acid sequence of at least about amino acids 203 to 736 of SEQ ID NO: 23, vp3 proteins produced from a sequence comprising at least nucleotides 607 to 2211 of SEQ ID NO: 24, or vp3 proteins produced from a nucleic acid sequence at least 70% identical to at least nucleotides 607 to 2211 of SEQ ID NO: 24 which encodes the predicted amino acid sequence of at least about amino acids 203 to 736 of SEQ ID NO: 23; and/or (2) a heterogeneous population of vp1 proteins which are the product of a nucleic acid sequence encoding the amino acid sequence of SEQ ID NO: 23, a heterogeneous population of vp2 proteins which are the product of a nucleic acid sequence encoding the amino acid sequence of at least about amino acids 138 to 736 of SEQ ID NO: 23, and a heterogeneous population of vp3 proteins which are the product of a nucleic acid sequence encoding at least amino acids 203 to 736 of SEQ ID NO: 23, wherein: the vp1, vp2 and vp3 proteins contain subpopulations with amino acid modifications comprising at least two highly deamidated asparagines (N) in asparagine-glycine pairs in SEQ ID NO: 23 and optionally further comprising subpopulations comprising other deamidated amino acids, wherein the deamidation results in an amino acid change;
(xiii) (1) AAV3B.AR2.14 capsid proteins comprising: a heterogeneous population of AAV3B.AR2.14 vp1 proteins selected from: vp1 proteins produced by expression from a nucleic acid sequence which encodes the predicted amino acid sequence of 1 to 736 of SEQ ID NO: 25, vp1 proteins produced from SEQ ID NO: 26, or vp1 proteins produced from a nucleic acid sequence at least 70% identical to SEQ ID NO: 26 which encodes the predicted amino acid sequence of 1 to 736 of SEQ ID NO: 25, a heterogeneous population of AAV3B.AR2.14 vp2 proteins selected from: vp2 proteins produced by expression from a nucleic acid sequence which encodes the predicted amino acid sequence of at least about amino acids 138 to 736 of SEQ ID NO: 25, vp2 proteins produced from a sequence comprising at least nucleotides 412 to 2211 of SEQ ID NO: 26, or vp2 proteins produced from a nucleic acid sequence at least 70% identical to at least nucleotides 412 to 2211 of SEQ ID NO: 26 which encodes the predicted amino acid sequence of at least about amino acids 138 to 736 of SEQ ID NO: 25, a heterogeneous population of AAV3B.AR2.14 vp3 proteins selected from: vp3 proteins produced by expression from a nucleic acid sequence which encodes the predicted amino acid sequence of at least about amino acids 203 to 736 of SEQ ID NO: 25, vp3 proteins produced from a sequence comprising at least nucleotides 607 to 2211 of SEQ ID NO: 26, or vp3 proteins produced from a nucleic acid sequence at least 70% identical to at least nucleotides 607 to 2211 of SEQ ID NO: 26 which encodes the predicted amino acid sequence of at least about amino acids 203 to 736 of SEQ ID NO: 25; and/or (2) a heterogeneous population of vp1 proteins which are the product of a nucleic acid sequence encoding the amino acid sequence of SEQ ID NO: 25, a heterogeneous population of vp2 proteins which are the product of a nucleic acid sequence encoding the amino acid sequence of at least about amino acids 138 to 736 of SEQ ID NO: 25, and a heterogeneous population of vp3 proteins which are the product of a nucleic acid sequence encoding at least amino acids 203 to 736 of SEQ ID NO: 25, wherein: the vp1, vp2 and vp3 proteins contain subpopulations with amino acid modifications comprising at least two highly deamidated asparagines (N) in asparagine-glycine pairs in SEQ ID NO: 25 and optionally further comprising subpopulations comprising other deamidated amino acids, wherein the deamidation results in an amino acid change;
(xiv) (1) AAV3B.AR2.15 capsid proteins comprising: a heterogeneous population of AAV3B.AR2.15 vp1 proteins selected from: vp1 proteins produced by expression from a nucleic acid sequence which encodes the predicted amino acid sequence of 1 to 736 of SEQ ID NO: 27, vp1 proteins produced from SEQ ID NO: 28, or vp1 proteins produced from a nucleic acid sequence at least 70% identical to SEQ ID NO: 28 which encodes the predicted amino acid sequence of 1 to 736 of SEQ ID NO: 27, a heterogeneous population of AAV3B.AR2.15 vp2 proteins selected from: vp2 proteins produced by expression from a nucleic acid sequence which encodes the predicted amino acid sequence of at least about amino acids 138 to 736 of SEQ ID NO: 27, vp2 proteins produced from a sequence comprising at least nucleotides 412 to 2211 of SEQ ID NO: 28, or vp2 proteins produced from a nucleic acid sequence at least 70% identical to at least nucleotides 412 to 2211 of SEQ ID NO: 28 which encodes the predicted amino acid sequence of at least about amino acids 138 to 736 of SEQ ID NO: 27, a heterogeneous population of AAV3B.AR2.15 vp3 proteins selected from: vp3 proteins produced by expression from a nucleic acid sequence which encodes the predicted amino acid sequence of at least about amino acids 203 to 736 of SEQ ID NO: 27, vp3 proteins produced from a sequence comprising at least nucleotides 607 to 2211 of SEQ ID NO: 28, or vp3 proteins produced from a nucleic acid sequence at least 70% identical to at least nucleotides 607 to 2211 of SEQ ID NO: 28 which encodes the predicted amino acid sequence of at least about amino acids 203 to 736 of SEQ ID NO: 27; and/or (2) a heterogeneous population of vp1 proteins which are the product of a nucleic acid sequence encoding the amino acid sequence of SEQ ID NO: 27, a heterogeneous population of vp2 proteins which are the product of a nucleic acid sequence encoding the amino acid sequence of at least about amino acids 138 to 736 of SEQ ID NO: 27, and a heterogeneous population of vp3 proteins which are the product of a nucleic acid sequence encoding at least amino acids 203 to 736 of SEQ ID NO: 27, wherein: the vp1, vp2 and vp3 proteins contain subpopulations with amino acid modifications comprising at least two highly deamidated asparagines (N) in asparagine-glycine pairs in SEQ ID NO: 27 and optionally further comprising subpopulations comprising other deamidated amino acids, wherein the deamidation results in an amino acid change;
(xv) (1) AAV3B.AR2.16 capsid proteins comprising: a heterogeneous population of AAV3B.AR2.16 vp1 proteins selected from: vp1 proteins produced by expression from a nucleic acid sequence which encodes the predicted amino acid sequence of 1 to 736 of SEQ ID NO: 29, vp1 proteins produced from SEQ ID NO: 30, or vp1 proteins produced from a nucleic acid sequence at least 70% identical to SEQ ID NO: 30 which encodes the predicted amino acid sequence of 1 to 736 of SEQ ID NO: 29, a heterogeneous population of AAV3B.AR2.16 vp2 proteins selected from: vp2 proteins produced by expression from a nucleic acid sequence which encodes the predicted amino acid sequence of at least about amino acids 138 to 736 of SEQ ID NO: 29, vp2 proteins produced from a sequence comprising at least nucleotides 412 to 2211 of SEQ ID NO: 30, or vp2 proteins produced from a nucleic acid sequence at least 70% identical to at least nucleotides 412 to 2211 of SEQ ID NO: 30 which encodes the predicted amino acid sequence of at least about amino acids 138 to 736 of SEQ ID NO: 29, a heterogeneous population of AAV3B.AR2.16 vp3 proteins selected from: vp3 proteins produced by expression from a nucleic acid sequence which encodes the predicted amino acid sequence of at least about amino acids 203 to 736 of SEQ ID NO: 29, vp3 proteins produced from a sequence comprising at least nucleotides 607 to 2211 of SEQ ID NO: 30, or vp3 proteins produced from a nucleic acid sequence at least 70% identical to at least nucleotides 607 to 2211 of SEQ ID NO: 30 which encodes the predicted amino acid sequence of at least about amino acids 203 to 736 of SEQ ID NO: 29; and/or (2) a heterogeneous population of vp1 proteins which are the product of a nucleic acid sequence encoding the amino acid sequence of SEQ ID NO: 29, a heterogeneous population of vp2 proteins which are the product of a nucleic acid sequence encoding the amino acid sequence of at least about amino acids 138 to 736 of SEQ ID NO: 29, and a heterogeneous population of vp3 proteins which are the product of a nucleic acid sequence encoding at least amino acids 203 to 736 of SEQ ID NO: 29, wherein: the vp1, vp2 and vp3 proteins contain subpopulations with amino acid modifications comprising at least two highly deamidated asparagines (N) in asparagine-glycine pairs in SEQ ID NO: 29 and optionally further comprising subpopulations comprising other deamidated amino acids, wherein the deamidation results in an amino acid change: or
(xvi) (1) AAV3B.AR2.17 capsid proteins comprising: a heterogeneous population of AAV3B.AR2.17 vp1 proteins selected from: vp1 proteins produced by expression from a nucleic acid sequence which encodes the predicted amino acid sequence of 1 to 736 of SEQ ID NO: 31, vp1 proteins produced from SEQ ID NO: 32, or vp1 proteins produced from a nucleic acid sequence at least 70% identical to SEQ ID NO: 32 which encodes the predicted amino acid sequence of 1 to 736 of SEQ ID NO: 31, a heterogeneous population of AAV3B.AR2.17 vp2 proteins selected from: vp2 proteins produced by expression from a nucleic acid sequence which encodes the predicted amino acid sequence of at least about amino acids 138 to 736 of SEQ ID NO: 31, vp2 proteins produced from a sequence comprising at least nucleotides 412 to 2211 of SEQ ID NO: 32, or vp2 proteins produced from a nucleic acid sequence at least 70% identical to at least nucleotides 412 to 2211 of SEQ ID NO: 32 which encodes the predicted amino acid sequence of at least about amino acids 138 to 736 of SEQ ID NO: 31, a heterogeneous population of AAV3B.AR2.17 vp3 proteins selected from: vp3 proteins produced by expression from a nucleic acid sequence which encodes the predicted amino acid sequence of at least about amino acids 203 to 736 of SEQ ID NO: 31, vp3 proteins produced from a sequence comprising at least nucleotides 607 to 2211 of SEQ ID NO: 32, or vp3 proteins produced from a nucleic acid sequence at least 70% identical to at least nucleotides 607 to 2211 of SEQ ID NO: 32 which encodes the predicted amino acid sequence of at least about amino acids 203 to 736 of SEQ ID NO: 31; and/or (2) a heterogeneous population of vp1 proteins which are the product of a nucleic acid sequence encoding the amino acid sequence of SEQ ID NO: 31, a heterogeneous population of vp2 proteins which are the product of a nucleic acid sequence encoding the amino acid sequence of at least about amino acids 138 to 736 of SEQ ID NO: 31, and a heterogeneous population of vp3 proteins which are the product of a nucleic acid sequence encoding at least amino acids 203 to 736 of SEQ ID NO: 31, wherein: the vp1, vp2 and vp3 proteins contain subpopulations with amino acid modifications comprising at least two highly deamidated asparagines (N) in asparagine-glycine pairs in SEQ ID NO: 31 and optionally further comprising subpopulations comprising other deamidated amino acids, wherein the deamidation results in an amino acid change; and
(B) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell.

13-27. (canceled)

28. A method of transducing liver tissue, the method comprising administering to a subject the rAAV according to claim 1.

29. A method of generating the recombinant adeno-associated virus (AAV) according to claim 12, the method comprising culturing a host cell containing: (a) a molecule encoding an AAV capsid protein of AAV3B.AR2.01 (SEQ ID NO: 2), AAV3B.AR2.02 (SEQ ID NO: 4), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.08 (SEQ ID NO: 16), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), AAV3B.AR2.16 (SEQ ID NO: 30), or AAV3B.AR2.17 (SEQ ID NO: 32); (b) a functional rep gene; (c) a minigene comprising AAV inverted terminal repeats (ITRs) and a transgene; and (d) sufficient helper functions to permit packaging of the minigene into the AAV capsid protein.

30. A host cell transfected with the rAAV according to claim 1.

31. A composition comprising at least the rAAV according to claim 1 and a physiologically compatible carrier, buffer, adjuvant, and/or diluent.

32. (canceled)

33. A nucleic acid molecule comprising a nucleic acid sequence encoding an AAV capsid protein, wherein said nucleic acid sequence is selected from AAV3B.AR2.01 (SEQ ID NO: 2), AAV3B.AR2.02 (SEQ ID NO: 4), AAV3B.AR2.03 (SEQ ID NO: 6), AAV3B.AR2.04 (SEQ ID NO: 8), AAV3B.AR2.05 (SEQ ID NO: 10), AAV3B.AR2.06 (SEQ ID NO: 12), AAV3B.AR2.07 (SEQ ID NO: 14), AAV3B.AR2.08 (SEQ ID NO: 16), AAV3B.AR2.10 (SEQ ID NO: 18), AAV3B.AR2.11 (SEQ ID NO: 20), AAV3B.AR2.12 (SEQ ID NO: 22), AAV3B.AR2.13 (SEQ ID NO: 24), AAV3B.AR2.14 (SEQ ID NO: 26), AAV3B.AR2.15 (SEQ ID NO: 28), AAV3B.AR2.16 (SEQ ID NO: 30), or AAV3B.AR2.17 (SEQ ID NO: 32).

34. The nucleic acid molecule according to claim 33, wherein said molecule further comprises an AAV sequence encoding an AAV capsid protein and a functional AAV rep protein.

35. The nucleic acid molecule according to claim 33, wherein said molecule is a plasmid.

36. A host cell transfected with a nucleic acid molecule according to claim 33.

37. A method of delivering a transgene to a cell, said method comprising the step of contacting the cell with the rAAV according to claim 1, wherein said rAAV comprises the transgene.

38-39. (canceled)

40. A method of delivering a transgene to a cell, said method comprising the step of contacting the cell with the rAAV according to claim 8, wherein said rAAV comprises the transgene.

41. A method of delivering a transgene to a cell, said method comprising the step of contacting the cell with the rAAV according to claim 12, wherein said rAAV comprises the transgene.


SEQUENCE LISTING (46 sequences)

SEQ ID NO: 1 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQRNGKAPTIGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 2 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagcgcaacggcaaagctcccaccatagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 3 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNLQNRNTAPTTETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacctccagaaccgcaacacagctcccaccacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 5 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVATDNQNRSKAPIIGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 6 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaccgacaaccagaaccgcagcaaagctcccatcatagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 7 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQRSSAAPNTGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 8 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaacaacctccagcgcagcagcgcagctcccaacacagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 9 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQSNSKAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagagcaacagcaaagctcccaccacacggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 11 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQHRNAAPNTETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 12 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagcaccgcaacgcagctcccaacacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 13 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVATDLQNRGKAPNTETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 14 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaccgacctccagaaccgcggcaaagctcccaacacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 15 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADDIQHRSKAPNIQTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 16 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacgacatccagcaccgcagcaaagctcccaacatacagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 17 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNIQSSSKAPITETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 18 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaacaacatccagagcagcagcaaagctcccatcacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 19 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSRNEAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 20 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaacaacctccagagccgcaacgaagctcccaccacacggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 21 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVATNIQRNGTAPITGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 22 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaccaacatccagcgcaacggcacagctcccatcacagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 23 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVATNLQRSGTAPITETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 24 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaccaacctccagcgcagcggcacagctcccatcacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 25 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQSSNKAPIIGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 26 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagagcagcaacaaagctcccatcatagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 27 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQRSGAAPITGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 28 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagcgcagcggcgcagctcccatcacagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 29 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVAANIQRNSTAPNTETVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 30 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagccaacatccagcgcaacagcacagctcccaacacagagactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 31 - protein, 736 aa, Artificial Sequence
  synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVADNIQRNSKAPTIGTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 32 - DNA, 2211 bp, Artificial Sequence
  synthetic construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcagacaacatccagcgcaacagcaaagctcccaccatagggactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 33 - protein, 736 aa, adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 34 - DNA, 2211 bp, adeno-associated virus 3B
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 35 - DNA, 99 bp, Artificial Sequence
  primer sequence
cggtcacgtctcccaacagagcagtatggaactgtcgcarmcvacmwccagvrcmrcrrcrmagctcccahcabasrgacagtcaatgatcagggagcc

SEQ ID NO: 36 - DNA, 59 bp, Artificial Sequence
  primer sequence
gccagtcgtctccggtcttgccacaccatgccaggtaaggctccctgatcattgactgt

SEQ ID NO: 37 - DNA, 39 bp, Artificial Sequence
  primer sequence
cggtcacgtctcccaacagagcagtatggaactgtcgca

SEQ ID NO: 38 - DNA, 38 bp, Artificial Sequence
  primer sequence
gccagtcgtctccggtcttgccacaccatgccaggtaa

SEQ ID NO: 39 - DNA, 25 bp, Artificial Sequence
  primer sequence
ggcgaacagcggacaccgatatgaa

SEQ ID NO: 40 - DNA, 25 bp, Artificial Sequence
  primer sequence
ggctctcgtcgcgtgagaatgagaa

SEQ ID NO: 41 - DNA, 22 bp, Artificial Sequence
  miRNA target sequence
agtgaattctaccagtgccata

SEQ ID NO: 42 - DNA, 23 bp, Artificial Sequence
  miRNA target sequence
agcaaaaatgtgctagtgccaaa

SEQ ID NO: 43 - DNA, 24 bp, Artificial Sequence
  miRNA target sequence
agtgtgagttctaccattgccaaa

SEQ ID NO: 44 - DNA, 23 bp, Artificial Sequence
  miRNA target sequence
agggattcctgggaaaactggac

SEQ ID NO: 45 - protein, 9 aa, Artificial Sequence
  synthetic construct
LAGLIDADG

SEQ ID NO: 46 - DNA, 22 bp, Artificial Sequence
  target recognition sequence
caaaacgtcgtgagacagtttg